(19) United States
(12) Patent Application Publication
(10) Pub. No.: US 2011/0178134 A1
(43) Pub. Date: Jul. 21, 2011

(54) Novel phenyl-substituted imidazolidines, process for preparation thereof, medicaments comprising said compounds and use thereof

(75) Inventors: Gerhard JAEHNE, Frankfurt (DE); Siegfried STENGELIN, Eppstein-Bremthal (DE); Matthias GOSSEL, Hofheim (DE); Thomas KLABUNDE, Frankfurt (DE); Irvin WINKLER, Liederbach (DE); Antony BIGOT, Massy (FR); Anita DIU-HERCEND, Charenton Le Pont (FR); Gilles TIRABOSCHI, Montgeron (FR)

(73) Assignee: SANOFI-AVENTIS, Paris (FR)

(21) Appl. No.: 12/852,038

(22) Filed: Aug. 6, 2010

Related U.S. Application Data
(63) Continuation of application No. PCT/EP2009/000588, filed on Jan. 30, 2009.

(30) Foreign Application Priority Data
Feb. 7, 2008 (EP) 08290133.1

(51) Int. Cl. A61K 31/4439; C07D 233/02; C07D 401/06; A61K 31/4166; A61P 3/04; A61P 3/10; A61P 25/00; A61P 25/28; A61P 25/32; A61P 25/34; A61P 25/18

(52) U.S. Cl. 514/341; 548/321.1; 546/274.4; 514/391

(57) ABSTRACT

The invention relates to compounds of formula (I) wherein the groups have stated meanings, and to their physiologically compatible salts. Said compounds are suitable, for example, as anti-obesity drugs and for treating cardiometabolic syndrome.

The invention relates to imidazolidinediones which are substituted by substituted phenyl on the imide nitrogen (N3) of the imidazolidine-2,4-dione system and to the physiologically compatible salts thereof.

Imidazolidine-2,4-diones with anti-androgenic action and the use thereof for treatment of neoplasias of the prostate have already been described (U.S. Pat. No. 5,411,981).

It was an object of the invention to provide compounds which display a therapeutically utilizable action. More particularly, it was an object of the invention to find novel compounds which are suitable for treatment of metabolic syndrome, of type II diabetes and of obesity.

The invention therefore relates to compounds of the formula I

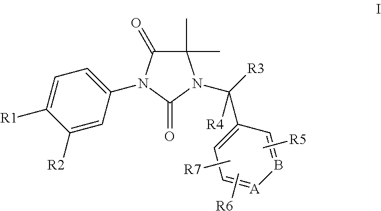

in which
R1 is CN, NO₂ or halogen;
R2 is CF₃ or halogen;
A, B are each independently CH, N;
R3, R4 are each independently hydrogen, (C₁-C₁₂)-alkyl, ((C₆-C₁₂)-aryl, (C₁-C₁₂)-alkylene-(C₆-C₁₂)-aryl, where (C₁-C₁₂)-alkyl, (C₆-C₁₂)-aryl, (C₁-C₁₂)-alkylene-(C₆-C₁₂)-aryl may each be up to trisubstituted independently by halogen, CN, CF₃;
R5, R6, R7 are each independently H, F, Cl, Br, CN, CF₃, SF₅, OCF₃, NO₂, S(O)ₘ[(C₁-C₆)-alkyl], S(O)ₘ[(C₃-C₉)-cycloalkyl], S(O)ₘCF₃, (C₁-C₆)-alkyl, (C₁-C₆)-alkoxy, (C₂-C₆)-alkenyl, (C₂-C₆)-alkenyloxy, (C₂-C₆)-alkynyl, (C₂-C₆)-alkynyloxy, OH, SH, W—COO—[(C₁-C₁₂)-alkyl], —O(C═O)—(C₆-C₁₂)-aryl, W—COOH, W—CONH₂, W—CO—NH[(C₁-C₆)-alkyl],
   W—CO—N[(C₁-C₆)-alkyl]₂, W—CO—NH[(C₃-C₉)-cycloalkyl], W—CO—N[(C₃-C₉)-cycloalkyl]₂, W—CO—NH—CN, W—CO—NH—CHR8-CO—R9 W—CO—R10, W—CO—NH—C(═NH)NH₂, W—CO—NH—C(═NH)NH[(C₁-C₆)-alkyl], W—CO—NH—C(═NH)N[(C₁-C₆)-alkyl]₂, (C₁-C₈)-acyl, (C₁-C₇)-acyloxy, W—C(═NH)NH₂, W—C(═NH)NHOH, W—C(═N—SO₂—NH₂)NH₂, W—C(═N—SO₂—CF₃)NH₂, W—C[═N—SO₂—(C₁-C₆)-alkyl]NH₂, W—C[═N—SO₂—(C₃-C₉)-cycloalkyl]NH₂, W—C(═N—SO₂-aryl)NH₂, NH₂, NH—(C₁-C₁₂)-alkyl, N—[(C₁-C₁₂)-alkyl]₂, W—NH—C(═NH)NH₂, W—NH—C(═NH)—NH[(C₁-C₆)-alkyl], W—NH—C(═NH)N[(C₁-C₆)-alkyl]₂, W—NH—CO—NH₂, W—NH—CO—NH[(C₁-C₆)-alkyl], W—NH—CO—N[(C₁-C₆)-alkyl]₂, W—NH—CO—NH[(C₃-C₉)-cycloalkyl], W—NH—CO—N[(C₃-C₉)-cycloalkyl]₂, W—NH—CO—NH—[(C₁-C₆)-alkyl]-CO—O—[(C₁-C₆)-alkyl], W—NH—CO—NH—[(C₁-C₆)-alkyl]-CO—NH₂, W—NH—CO—NH—SO₂—(C₁-C₆)-alkyl, W—NH—CO—NH—SO₂-[(C₃-C₉)-cycloalkyl], W—NH—CO—NH—CO—(C₁-C₆)-alkyl, W—NH—CO—NH—CO—[(C₃-C₉)-cycloalkyl], W—NH—C(═NH)—NH—C(═NH)—NH₂, W—NH—C(═NH)—NH—C(═NH)—NH[(C₁-C₆)-alkyl], W—NH—C(═NH)—NH—C(═NH)—N[(C₁-C₆)-alkyl]₂, W—NH—W—SO₂—NH₂, W—NH—W—SO₂—NH[(C₁-C₆)-alkyl], W—NH—W—SO₂—N[(C₁-C₆)-alkyl]₂, W—NH—W—SO₂—NH[(C₃-C₉)-cycloalkyl], W—NH—W—SO₂—N[(C₃-C₉)-cycloalkyl]₂, W—NH—W—SO₂—NH—CO—O[(C₁-C₆)-alkyl], W—NH—W—SO₂—NH—CO—NH₂, W—O—SO₂—NH₂, W—O—W—COOH, W—O—W—CONH₂, W—SO₂—NH₂, W—SO₂—NH[(C₁-C₆)-alkyl], W—SO₂—N[(C₁-C₆)-alkyl]₂, W—SO₂—NH[(C₃-C₉)-cycloalkyl], W—SO₂—N[(C₃-C₉)-cycloalkyl]₂, W—SO₃H, W—NH—W—SO₃H, W—SO₂—NH—CO—NH₂, W—SO₂—NH—CO—NH[(C₁-C₆)-alkyl], W—SO₂—NH—CO—N[(C₁-C₆)-alkyl]₂, W—SO₂—NH—CO—NH[(C₃-C₉)-cycloalkyl], W—SO₂—NH—CO—N[(C₃-C₉)-cycloalkyl]₂, W—P(O)(OH)[O—(C₁-C₆)-alkyl], W—P(O)[O—(C₁-C₆)-alkyl]₂, W—P(O)(OH)(O—CH₂-aryl), W—P(O)(O—CH₂-aryl)₂, W—P(O)(OH)₂, (C₆-C₁₂)-aryl, O—(C₆-C₁₂)-aryl, O—(C₁-C₁₂)-alkylene-(C₆-C₁₂)-aryl, S(O)ₘ—C₆-C₁₂)-aryl, tri(C₁-C₁₂)-alkylsilyl, where the alkyl has from 1 to 6 carbon atoms;
m is 0, 1, 2;
W is a bond or (C₁-C₆)-alkyl;
R8 is H, (C₁-C₆)-alkyl, where the alkyl group may be substituted by OH, SH, SCH₃, aryl, 4-hydroxyaryl, heteroaryl, NH₂, NH—C(═NH)NH₂, COOH, CO—O(C₁-C₆)-alkyl, CONH₂;
R9 is OH, NH₂, NH—(C₁-C₁₂)-alkyl, N[(C₁-C₁₂)-alkyl]₂, NH—(C₃-C₉)-cycloalkyl, N[(C₃-C₉)-cycloalkyl]₂;
R10 is NH—(C₁-C₆)-alkyl-SO₃H, NH—(C₁-C₆)-alkyl-SO₂NH₂, NH—(C₁-C₆)-alkyl-SO₂—(C₁-C₆)-alkyl, NH—(C₁-C₆)-alkyl-SO₂—(C₃-C₉)-cycloalkyl, NH—(C₁-C₆)-alkyl-SO₂—CF₃,

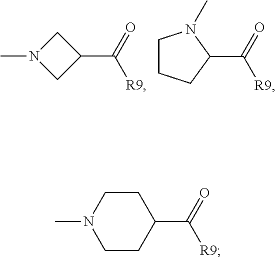

and the physiologically compatible salts thereof.

Preference is given to compounds of the formula I in which one or more radicals are defined as follows:
R1 is CN or halogen;
R2 is CF₃ or halogen;
A, B are each independently CH, N;
R3, R4 are each independently hydrogen, (C₁-C₁₂)-alkyl, ((C₆-C₁₂)-aryl, (C₁-C₁₂)-alkylene-(C₆-C₁₂)-aryl, where (C₁-C₁₂)-alkyl, (C₆-C₁₂)-aryl, (C₁-C₁₂)-alkylene-(C₆-C₁₂)-aryl may each be up to trisubstituted independently by halogen, CN, CF₃;
R5 F, Cl, Br, CN, CF₃, SF₅, OCF₃, NO₂, S(O)ₘ[(C₁-C₆)-alkyl], S(O)ₘ[(C₃-C₉)-cycloalkyl], S(O)ₘCF₃, (C₁-C₆)-alkyl, (C₁-C₆)-alkoxy, (C₂-C₆)-alkenyl, (C₂-C₆)-alkenyloxy,
   (C₂-C₆)-alkynyl, (C₂-C₆)-alkynyloxy, OH, SH, W—COO—[(C₁-C₁₂)-alkyl], —O(C═O)—(C₆-C₁₂)-aryl, W—COOH, W—CONH₂, W—CO—NH[(C₁-C₆)-alkyl], W—CO—N[(C₁-C₆)-alkyl]₂, W—CO—NH[(C₃-C₉)-cycloalkyl], W—CO—N[(C₃-C₉)-cycloalkyl]₂, W—CO—NH—CN, W—CO—NH—CHR8-CO—R9, W—CO—R10, W—CO—NH—C(═NH)NH₂, W—CO—NH—C(═NH)NH[(C₁-C₆)-alkyl], W—CO—NH—C(═NH)N[(C₁-C₆)-alkyl]₂, (C₁-C₈)-acyl, (C₁-C₇)-acyloxy, W—C(═NH)NH₂, W—C(═NH)NHOH, W—C(═N—SO₂—NH₂)NH₂, W—C(═N—SO₂—CF₃)NH₂, W—C[═N—SO₂—(C₁-C₆)-alkyl]NH₂, W—C[═N—SO₂—(C₃-C₉)-cycloalkyl]NH₂, W—C(═N—SO₂-aryl)NH₂, NH₂, NH—(C₁-C₁₂)-alkyl, N—[(C₁-C₁₂)-alkyl]₂, W—NH—C(═NH)NH₂, W—NH—C(═NH)NH[(C₁-C₆)-alkyl], W—NH—C(═NH)N[(C₁-C₆)-alkyl]₂, W—NH—CO—NH₂, W—NH—CO—NH[(C₁-C₆)-alkyl], W—NH—CO—N[(C₁-C₆)-alkyl]₂, W—NH—CO—NH[(C₃-C₉)-cycloalkyl], W—NH—CO—N[(C₃-C₉)-cycloalkyl]₂, W—NH—CO—NH—[(C₁-C₆)-alkyl]-CO—O—[(C₁-C₆)-alkyl], W—NH—CO—NH—[(C₁-C₆)-alkyl]-CO—NH₂, W—NH—CO—NH—SO₂—(C₁-C₆)-alkyl, W—NH—CO—NH—SO₂-[(C₃-C₉)-cycloalkyl], W—NH—CO—NH—CO—(C₁-C₆)-alkyl, W—NH—CO—NH—CO—[(C₃-C₉)-cycloalkyl], W—NH—C(═NH)—NH—C(═NH)—NH₂, W—NH—C(═NH)—NH—C(═NH)—NH[(C₁-C₆)-alkyl], W—NH—C(═NH)—NH—C(═NH)—N[(C₁-C₆)-alkyl]₂, W—NH—W—SO₂—NH₂, W—NH—W—SO₂—NH[(C₁-C₆)-alkyl], W—NH—W—SO₂—N[(C₁-C₆)-alkyl]₂, W—NH—W—SO₂—NH[(C₃-C₉)-cycloalkyl], W—NH—W—SO₂—N[(C₃-C₉)-cycloalkyl]₂, W—NH—W—SO₂—NH—CO—O[(C₁-C₆)-alkyl], W—NH—W—SO₂—NH—CO—NH₂, W—O—SO₂—NH₂, W—O—W—COOH, W—O—W—CONH₂, W—SO₂—NH₂, W—SO₂—NH[(C₁-C₆)-alkyl], W—SO₂—N[(C₁-C₆)-alkyl]₂, W—SO₂—NH[(C₃-C₉)-cycloalkyl], W—SO₂—N[(C₃-C₉)-cycloalkyl]₂, W—SO₃H, W—NH—W—SO₃H, W—SO₂—NH—CO—NH₂, W—SO₂—NH—CO—NH[(C₁-C₆)-alkyl], W—SO₂—NH—CO—N[(C₁-C₆)-alkyl]₂, W—SO₂—NH—CO—NH[(C₃-C₉)-cycloalkyl], W—SO₂—NH—CO—N[(C₃-C₉)-cycloalkyl]₂, W—P(O)(OH)[O—(C₁-C₆)-alkyl], W—P(O)[o—(C₁-C₆)-alkyl]₂, W—P(O)(OH)(O—CH₂-aryl), W—P(O)(O—CH₂-aryl)₂, W—P(O)(OH)₂, (C₆-C₁₂)-aryl, O—(C₆-C₁₂)-aryl, O—(C₁-C₁₂)-alkylene-(C₆-C₁₂)-aryl, S(O)ₘ—(C₆-C₁₂)-aryl, tri(C₁-C₁₂)-alkylsilyl, where the alkyl has from 1 to 6 carbon atoms;
R6, R7 are each independently H, halogen, CN, CF₃, SF₅, OCF₃, S(O)ₘ[(C₁-C₆)-alkyl], S(O)ₘ[(C₃-C₉)-cycloalkyl], NO₂, S(O)ₘCF₃, (C₁-C₆)-alkyl, (C₁-C₆)-alkoxy, (C₂-C₆)-alkenyl, (C₂-C₆)-alkenyloxy,
   (C₂-C₆)-alkynyl, (C₂-C₆)-alkynyloxy, OH, SH, W—COO—[(C₁-C₁₂)-alkyl], —O(C═O)—(C₆-C₁₂)-aryl, W—COOH, W—CONH₂, W—CO—NH[(C₁-C₆)-alkyl], W—CO—N[(C₁-C₆)-alkyl]₂, W—CO—NH[(C₃-C₉)-cycloalkyl], W—CO—N[(C₃-C₉)-cycloalkyl]₂, W—CO—NH—CN, W—CO—NH—CHR8-CO—R9, W—CO—R10, W—CO—NH—C(═NH)NH₂, W—CO—NH—C(═NH)NH[(C₁-C₆)-alkyl], W—CO—NH—C(═NH)N[(C₁-C₆)-alkyl]₂, (C₁-C₈)-acyl, (C₁-C₇)-acyloxy, W—C(═NH)NH₂, W—C(═NH)NHOH, W—C(═N—SO₂—NH₂)NH₂, W—C(═N—SO₂—CF₃)NH₂, W—C[═N—SO₂—(C₁-C₆)-alkyl]NH₂, W—C[═N—SO₂—(C₃-C₉)-cycloalkyl]NH₂, W—C(═N—SO₂-aryl)NH₂, NH₂, NH—(C₁-C₁₂)-alkyl, N—[(C₁-C₁₂)-alkyl]₂, W—NH—C(═NH)NH₂, W—NH—C(═NH)NH[(C₁-C₆)-alkyl], W—NH—C(═NH)N[(C₁-C₆)-alkyl]₂, W—NH—CO—NH₂, W—NH—CO—NH[(C₁-C₆)-alkyl], W—NH—CO—N[(C₁-C₆)-alkyl]₂, W—NH—CO—NH[(C₃-C₉)-cycloalkyl], W—NH—CO—N[(C₃-C₉)-cycloalkyl]₂, W—NH—CO—NH—[(C₁-C₆)-alkyl]-CO—O—[(C₁-C₆)-alkyl], W—NH—CO—NH—[(C₁-C₆)-alkyl]-CO—NH₂, W—NH—CO—NH—SO₂—(C₁-C₆)-alkyl, W—NH—CO—NH—SO₂—[(C₃-C₉)-cycloalkyl], W—NH—CO—NH—CO—(C₁-C₆)-alkyl, W—NH—CO—NH—CO—[(C₃-C₉)-cycloalkyl], W—NH—C(═NH)—NH—C(═NH)—NH₂, W—NH—C(═NH)—NH—C(═NH)—NH[(C₁-C₆)-alkyl], W—NH—C(═NH)—NH—C(═NH)—N[(C₁-C₆)-alkyl]₂, W—NH—W—SO₂—NH₂, W—NH—W—SO₂—NH[(C₁-C₆)-alkyl], W—NH—W—SO₂—N[(C₁-C₆)-alkyl]₂, W—NH—W—SO₂—NH[(C₃-C₉)-cycloalkyl], W—NH—W—SO₂—N[(C₃-C₉)-cycloalkyl]₂, W—NH—W—SO₂—NH—CO—O[(C₁-C₆)-alkyl], W—NH—W—SO₂—NH—CO—NH₂, W—O—SO₂—NH₂, W—O—W—COOH, W—O—W—CONH₂, W—SO₂—NH₂, W—SO₂—NH[(C₁-C₆)-alkyl], W—SO₂—N[(C₁-C₆)-alkyl]₂, W—SO₂—NH[(C₃-C₉)-cycloalkyl], W—SO₂—N[(C₃-C₉)-cycloalkyl]₂, W—SO₃H, W—NH—W—SO₃H, W—SO₂—NH—CO—NH₂, W—SO₂—NH—CO—NH[(C₁-C₆)-alkyl], W—SO₂—NH—CO—N[(C₁-C₆)-alkyl]₂, W—SO₂—NH—CO—NH[(C₃-C₉)-cycloalkyl], W—SO₂—NH—CO—N[(C₃-C₉)-cycloalkyl]₂, W—P(O)(OH)[O—(C₁-C₆)-alkyl], W—P(O)[O—(C₁-C₆)-alkyl]₂, W—P(O)(OH)(O—CH₂-aryl), W—P(O)(O—CH₂-aryl)₂, W—P(O)(OH)₂, (C₆-C₁₂)-aryl, O—(C₆-C₁₂)-aryl, O—(C₁-C₁₂)-alkylene-(C₆-C₁₂)-aryl, S(O)ₘ—(C₆-C₁₂)-aryl, tri(C₁-C₁₂)-alkylsilyl, where the alkyl has from 1 to 6 carbon atoms;
m is 0, 1, 2;
W is a bond or (C₁-C₆)-alkyl;
R8 is H, (C₁-C₆)-alkyl, where the alkyl group may be substituted by OH, SH, SCH₃, aryl, 4-hydroxyaryl, heteroaryl, NH₂, NH—C(═NH)NH₂, COOH, CO—O(C₁-C₆)-alkyl, CONH₂;
R9 is OH, NH₂, NH—(C₁-C₁₂)-alkyl, N[(C₁-C₁₂)-alkyl]₂, NH—(C₃-C₉)-cycloalkyl, N[(C₃-C₉)-cycloalkyl]₂;
R10 is NH—(C₁-C₆)-alkyl-SO₃H, NH—(C₁-C₆)-alkyl-SO₂NH₂, NH—(C₁-C₆)-alkyl-SO₂—(C₁-C₆)-alkyl, NH—(C₁-C₆)-alkyl-SO₂—(C₃-C₉)-cycloalkyl, NH—(C₁-C₆)-alkyl-SO₂—CF₃,

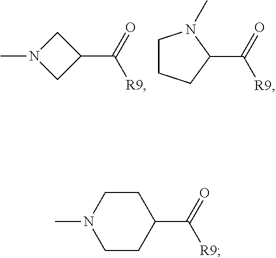

and the physiologically compatible salts thereof.

Particular preference is given to compounds of the formula I in which one or more radicals are defined as follows:
R1 is CN or halogen;
R2 is CF₃ or halogen;
A, B are each independently CH, N;
R3, R4 are each independently hydrogen, (C₁-C₁₂)-alkylene-(C₆-C₁₂)-aryl;
R5 is F, Cl, Br, CF₃, SF₅, OCF₃, S(O)₂[(C₁-C₆)-alkyl], (C₁-C₆)-alkyl, OH, —COOH, NH₂, —NH—CO—NH—[(C₁-C₆)-alkyl]-CO—O—[(C₁-C₆)-alkyl], —NH—SO₂—NH₂, —NH—SO₂—NH—CO—O[(C₁-C₆)-alkyl], (C₆-C₁₂)-aryl, O—(C₆-C₁₂)-aryl, O—(C₁-C₁₂)-alkylene-(C₆-C₁₂)-aryl, S(O)ₘ—(C₆-C₁₂)-aryl;
R6, R7 are each independently H, halogen, CN, CF₃, SF₅, OCF₃, S(O)ₘ[(C₁-C₆)-alkyl], S(O)ₘ[(C₃-C₉)-cycloalkyl], S(O)ₘCF₃, (C₁-C₆)-alkyl, (C₁-C₆)-alkoxy, (C₂-C₆)-alkenyl, (C₂-C₆)-alkenyloxy,
   (C₂-C₆)-alkynyl, (C₂-C₆)-alkynyloxy, OH, SH, W—COO—[(C₁-C₁₂)-alkyl], —O(C═O)—(C₆-C₁₂)-aryl, W—COOH, W—CONH₂, W—CO—NH[(C₁-C₆)-alkyl], W—CO—N[(C₁-C₆)-alkyl]₂, W—CO—NH[(C₃-C₉)-cycloalkyl], W—CO—N[(C₃-C₉)-cycloalkyl]₂, W—CO—NH—CN, W—CO—NH—CHR8-CO—R9, W—CO—R10, W—CO—NH—C(═NH)NH₂, W—CO—NH—C(═NH)NH[(C₁-C₆)-alkyl], W—CO—NH—C(═NH)N[(C₁-C₆)-alkyl]₂, (C₁-C₈)-acyl, (C₁-C₇)-acyloxy, W—C(═NH)NH₂, W—C(═NH)NHOH, W—C(═N—SO₂—NH₂)NH₂, W—C(═N—SO₂—CF₃)NH₂, W—C[═N—SO₂—(C₁-C₆)-alkyl]NH₂, W—C[═N—SO₂—(C₃-C₉)-cycloalkyl]NH₂, W—C(═N—SO₂-aryl)NH₂, NH₂, NH—(C₁-C₁₂)-alkyl, N—[(C₁-C₁₂)-alkyl]₂, W—NH—C(═NH)NH₂, W—NH—C(═NH)NH[(C₁-C₆)-alkyl], W—NH—C(═NH)N[(C₁-C₆)-alkyl]₂, W—NH—CO—NH₂, W—NH—CO—NH[(C₁-C₆)-alkyl], W—NH—CO—N[(C₁-C₆)-alkyl]₂, W—NH—CO—NH[(C₃-C₉)-cycloalkyl], W—NH—CO—N[(C₃-C₉)-cycloalkyl]₂, W—NH—CO—NH—[(C₁-C₆)-alkyl]-CO—O—[(C₁-C₆)-alkyl], W—NH—CO—NH—[(C₁-C₆)-alkyl]-CO—NH₂, W—NH—CO—NH—SO₂—(C₁-C₆)-alkyl, W—NH—CO—NH—SO₂—[(C₃-C₉)-cycloalkyl], W—NH—CO—NH—CO—(C₁-C₆)-alkyl, W—NH—CO—NH—CO—[(C₃-C₉)-cycloalkyl], W—NH—C(═NH)—NH—C(═NH)—NH₂, W—NH—C(═NH)—NH—C(═NH)—NH[(C₁-C₆)-alkyl], W—NH—C(═NH)—NH—C(═NH)—N[(C₁-C₆)-alkyl]₂, W—NH—W—SO₂—NH₂, W—NH—W—SO₂—NH[(C₁-C₆)-alkyl], W—NH—W—SO₂—N[(C₁-C₆)-alkyl]₂, W—NH—W—SO₂—NH[(C₃-C₉)-cycloalkyl], W—NH—W—SO₂—N[(C₃-C₉)-cycloalkyl]₂, W—NH—W—SO₂—NH—CO—O[(C₁-C₆)-alkyl], W—NH—W—SO₂—NH—CO—NH₂, W—O—SO₂—NH₂, W—O—W—COOH, W—O—W—CONH₂, W—SO₂—NH₂, W—SO₂—NH[(C₁-C₆)-alkyl], W—SO₂—N[(C₁-C₆)-alkyl]₂, W—SO₂—NH[(C₃-C₉)-cycloalkyl], W—SO₂—N[(C₃-C₉)-cycloalkyl]₂, W—SO₃H, W—NH—W—SO₃H, W—SO₂—NH—CO—NH₂, W—SO₂—NH—CO—NH[(C₁-C₆)-alkyl], W—SO₂—NH—CO—N[(C₁-C₆)-alkyl]₂, W—SO₂—NH—CO—NH[(C₃-C₉)-cycloalkyl], W—SO₂—NH—CO—N[(C₃-C₉)-cycloalkyl]₂, W—P(O)(OH)[O—(C₁-C₆)-alkyl], W—P(O)[O—(C₁-C₆)-alkyl]₂, W—P(O)(OH)(O—CH₂-aryl), W—P(O)(O—CH₂-aryl)₂, W—P(O)(OH)₂, (C₆-C₁₂)-aryl, O—(C₆-C₁₂)-aryl, O—(C₁-C₁₂)-alkylene-(C₆-C₁₂)-aryl, S(O)ₘ—(C₆-C₁₂)-aryl, tri(C₁-C₁₂)-alkylsilyl, where the alkyl has from 1 to 6 carbon atoms;
m is 0, 1, 2;
W is a bond or (C₁-C₆)-alkyl;
R8 is H, (C₁-C₆)-alkyl, where the alkyl group may be substituted by OH, SH, SCH₃, aryl, 4-hydroxyaryl, heteroaryl, NH₂, NH—C(═NH)NH₂, COOH, CO—O(C₁-C₆)-alkyl, CONH₂;
R9 is OH, NH₂, NH—(C₁-C₁₂)-alkyl, N[(C₁-C₁₂)-alkyl]₂, NH—(C₃-C₉)-cycloalkyl, N[(C₃-C₉)-cycloalkyl]₂;
R10 is NH—(C₁-C₆)-alkyl-SO₃H, NH—(C₁-C₆)-alkyl-SO₂NH₂, NH—(C₁-C₆)-alkyl-SO₂—(C₁-C₆)-alkyl, NH—(C₁-C₆)-alkyl-SO₂—(C₃-C₉)-cycloalkyl, NH—(C₁-C₆)-alkyl-SO₂—CF₃,

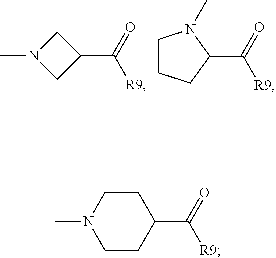

and the physiologically compatible salts thereof.

Very particular preference is given to compounds of the formula I in which one or more radicals are defined as follows:
R1 is CN or halogen;
R2 is CF₃ or halogen;
A, B are each independently CH, N;
R3, R4 are each independently hydrogen, (C₁-C₁₂)-alkylene-(C₆-C₁₂)-aryl;
R5 is F, Cl, Br, CF₃, SF₅, OCF₃, S(O)₂[(C₁-C₆)-alkyl], (C₁-C₆)-alkyl, OH, —COOH, NH₂, —NH—CO—NH—[(C₁-C₆)-alkyl]-CO—O—[(C₁-C₆)-alkyl], —NH—SO₂—NH₂, —NH—SO₂—NH—CO—O[(C₁-C₆)-alkyl], (C₆-C₁₂)-aryl, O—(C₆-C₁₂)-aryl, O—(C₁-C₁₂)-alkylene-(C₆-C₁₂)-aryl, S(O)ₘ—(C₆-C₁₂)-aryl;
R6, R7 are each independently H, halogen, CF₃, SF₅, OCF₃, S(O)₂[(C₁-C₆)-alkyl], (C₁-C₆)-alkyl, OH, —COOH,
   NH₂, —NH—CO—NH—[(C₁-C₆)-alkyl]-CO—O—[(C₁-C₆)-alkyl], —NH—SO₂—NH₂, —NH—SO₂—NH—CO—O[(C₁-C₆)-alkyl], (C₆-C₁₂)-aryl, O—(C₆-C₁₂)-aryl, O—(C₁-C₁₂)-alkylene-(C₆-C₁₂)-aryl, S(O)ₘ—(C₆-C₁₂)-aryl;
and the physiologically compatible salts thereof.

Very particular preference is further given to compounds of the formula I in which one or more radicals are defined as follows:
R1 is CN or halogen;
R2 is CF₃ or halogen;
A is CH;
B is CH, N;
R3, R4 are each independently hydrogen, (C₁-C₁₂)-alkylene-(C₆-C₁₂)-aryl;
R5 is SF₅, OCF₃, S(O)₂[(C₁-C₆)-alkyl], —NH—CO—NH—[(C₁-C₆)-alkyl]-CO—O—[(C₁-C₆)-alkyl], —NH—SO₂—NH₂, —NH—SO₂—NH—CO—O[(C₁-C₆)-alkyl], O—(C₁-C₁₂)-alkylene-(C₆-C₁₂)-aryl;
R6, R7 are each independently H, halogen, CF₃, SF₅, OCF₃, S(O)₂[(C₁-C₆)-alkyl], (C₁-C₆)-alkyl, OH, —COOH,
   NH₂, —NH—CO—NH—[(C₁-C₆)-alkyl]-CO—O—[(C₁-C₆)-alkyl], —NH—SO₂—NH₂, —NH—SO₂—NH—CO—O[(C₁-C₆)-alkyl], (C₆-C₁₂)-aryl, O—(C₆-C₁₂)-aryl, O—(C₁-C₁₂)-alkylene-(C₆-C₁₂)-aryl, S(O)ₘ—(C₆-C₁₂)-aryl;
and the physiologically compatible salts thereof.

In one embodiment, preference is given to compounds of the formula I in which R1 is CN.

In one embodiment, preference is given to compounds of the formula I in which R1 is NO₂.

In one embodiment, preference is given to compounds of the formula I in which R1 is halogen.

In one embodiment, preference is given to compounds of the formula I in which R2 is CF₃.

In one embodiment, preference is given to compounds of the formula I in which R1 is halogen.

In one embodiment, preference is given to compounds of the formula I in which A is CH.

In one embodiment, preference is given to compounds of the formula I in which A is N.

In one embodiment, preference is given to compounds of the formula I in which B is CH.

In one embodiment, preference is given to compounds of the formula I in which B is N.

In one embodiment, preference is given to compounds of the formula I in which A and B are each CH.

In one embodiment, preference is given to compounds of the formula I in which A is N and B is CH.

In one embodiment, preference is given to compounds of the formula I in which R5 is not H.

In one embodiment, preference is given to compounds of the formula I in which R5 and R6 are not H.

In one embodiment, preference is given to compounds of the formula I in which R5 is OCF₃.

In one embodiment, preference is given to compounds of the formula I in which R5 is SF₅.

The invention further provides both stereoisomer mixtures of the formula I and the pure stereoisomers of the formula I, and also diastereoisomer mixtures of the formula I and the pure diastereoisomers. The mixtures are separated, for example, by a chromatographic route.

The invention relates to compounds of the formula I in the form of their tautomers, racemates, racemic mixtures, stereoisomer mixtures, pure stereoisomers, diastereoisomer mixtures, pure diastereoisomers. The mixtures are separated, for example, by a chromatographic route.

Owing to their high water solubility, pharmaceutically acceptable salts are particularly suitable for medical applications compared to the starting or base compounds. These salts must have a pharmaceutically acceptable anion or cation. Suitable pharmaceutically acceptable acid addition salts of the inventive compounds are salts of inorganic acids, such as hydrochloric acid, hydrobromic acid, phosphoric acid, metaphosphoric acid, nitric acid and sulfuric acid, and also organic acids, for example acetic acid, benzenesulfonic acid, benzoic acid, citric acid, ethanesulfonic acid, fumaric acid, gluconic acid, glycolic acid, isethionic acid, lactic acid, lactobionic acid, maleic acid, malic acid, methanesulfonic acid, succinic acid, p-toluenesulfonic acid and tartaric acid. Suitable pharmaceutically acceptable basic salts are ammonium salts, alkali metal salts (such as sodium and potassium salts), alkaline earth metal salts (such as magnesium and calcium salts), trometamol (2-amino-2-hydroxymethyl-1,3-propanediol), diethanolamine, lysine or ethylenediamine.

Salts with a pharmaceutically unacceptable anion, for example trifluoroacetate, are also included within the scope of the invention as useful intermediates for the preparation or purification of pharmaceutically acceptable salts and/or for use in non-therapeutic applications, for example in vitro applications.

The inventive compounds may also be present in different polymorphic forms, for example as amorphous and crystalline polymorphic forms. All polymorphic forms of the inventive compounds are included within the scope of the invention and are a further aspect of the invention.

Hereinafter, all references to “compound(s) of the formula I” relate to compound(s) of the formula I as described above, and to their salts and solvates as described herein.

(C₁-C₁₂-Alkyl) is understood to mean a straight-chain or branched hydrocarbon chain having from one to twelve carbons, for example methyl, ethyl, isopropyl, tert-butyl, hexyl, dodecyl.

Halogen is understood to mean F, Cl or Br.

An aryl radical is understood to mean a phenyl, naphthyl, biphenyl, tetrahydronaphthyl, alpha- or beta-tetralonyl, indanyl or indan-1-onyl radical.

The aryl radicals may be mono- or polysubstituted by suitable groups as described above.

A heteroaryl radical is understood to mean aromatic rings and ring systems which, apart from carbon, also contain heteroatoms, for example nitrogen, oxygen or sulfur. This definition also includes ring systems in which the heteroaryl radical is fused to benzene rings. This likewise includes systems in which one or more CH group(s) has/have been replaced by CO═O or C═S, preferably CO═O.

Suitable heteroaryl radicals are, for example, furyl, imidazolyl, benzimidazolyl, indolyl, indolinyl, pyrimidinyl, pyridyl, pyrazinyl, pyrrolyl, thiazolyl, oxazolyl, thienyl, 1,2,3-triazolyl, 1,2,4-triazolyl, tetrazolyl, isoxazolyl, pyridazinyl, 1,3,5-triazinyl, 1,2,4-triazinyl; the 2H-pyridazin-3-one, dihydropyridazine-3,6-dione, imidazolidin-2-one, 1,3-dihydroimidazol-2-one, imidazolidin-2,5-dione, quinoline, isoquinoline, quinoxaline, quinazoline system.

The linkage to the heteroaryl radicals may be at any of the possible atoms; for example, pyridyl may be 2-, 3- or 4-pyridyl; thienyl may be 2- or 3-thienyl; furyl may be 2- or 3-furyl.

Also included are the corresponding N-oxides of these compounds, i.e., for example, 1-oxy-2-, -3- or -4-pyridyl.

The heteroaryl radicals may be mono- or polysubstituted by suitable groups as described above.

The invention also encompasses solvates or hydrates of the compounds of the formula I.

The compounds of the formula I are cannabinoid 1 receptor (CB1R) modulators and are, as such, suitable in humans and in animals for the treatment or for the prevention of diseases which are based on disruption of the endocannabinoid system.

For example, and without restriction, the compounds of the formula I are useful as psychotropic medicaments, especially for the treatment of psychiatric disorders including states of anxiety, depressions, disorders of the mind, insomnia, deliria, obsessive-compulsive neuroses, general psychoses, schizophrenia, attention deficit hyperactivity disorder (ADHD) in hyperkinetic children, and for the treatment of disorders in connection with the use of psychotropic substances, especially in the case of abuse of a substance and/or dependence on such a substance, including alcohol dependence and nicotine dependence, but also dependence on cocaine, methamphetamine and heroin (see, for example, Behavioural Pharmacology 2005, 16:275-296). Reviews of CBR1-mediated means of therapeutic intervention can be found, for example, in Ken Mackie: Annu Rev. Pharmacol. Toxicol. 46, 101-122 (2006), S. C. Black: Curr. Opin. Investig. Drugs 5, 389-394 (2004), V. Di Marzio et al.: Nat. Rev. Drug Discov. 3, 771-784 (2004), B. Le Foll et al.: J. Pharmacol. Exp. Ther. 312, 875-883 (2005) or L. Walter et al.: Br. J. Pharmacol. 141, 775-785 (2004).

The inventive compounds of the formula I may be used as medicaments for the treatment of migraine, stress, disorders of psychosomatic origin, panic attacks, epilepsy, disrupted movement, especially dyskinesias or Parkinson's disease, trembling and dystonia.

The inventive compounds of the formula I can also be used as medicaments for the treatment of disorders of memory, mental defects, especially for the treatment of age-related dementia, of Alzheimer's disease and for the treatment of reduced alertness or wakefulness.

In addition, it is also possible to use the compounds of the formula I as neuroprotectors, for the treatment of ischemia, cranial injuries and the treatment of neurodegenerative disorders, including chorea, Huntington's chorea, Tourette's syndrome.

The inventive compounds of the formula I can also be used as medicaments in the treatment of pain; this includes neuropathic pain, acute peripheral pain, chronic pain of inflammatory origin.

The inventive compounds of the formula I may also serve as medicaments for the treatment of eating disorders (for example binge eating disorders, anorexia and bulimia), for the treatment of addiction to confectionery, carbohydrates, drugs, alcohol or other addictive substances.

The inventive compounds of the formula I are particularly suitable for the treatment of obesity or of bulimia, and for the treatment of type II diabetes and also for the treatment of dyslipidemias and of metabolic syndrome. The inventive compounds of the formula I are therefore useful for the treatment of obesity and of the risks associated with obesity, especially the cardiovascular risks.

Moreover, the inventive compounds of the formula I may be used as medicaments for the treatment of gastrointestinal disorders, for the treatment of diarrhea, of gastric and intestinal ulcers, of vomiting, of bladder trouble and disorders of urination, of disorders of endocrine origin, of cardiovascular problems, of low blood pressure, of hemorrhagic shock, of septic shock, chronic liver cirrhosis, liver steatosis, of nonalcoholic steatohepatitis, of asthma, of Raynaud's syndrome, of glaucoma, of fertility problems, termination of pregnancy, early birth, inflammatory symptoms, disorders of the immune system, especially autoimmune and neuroinflammatory disorders, for example rheumatic inflammation of joints, reactive arthritis, of disorders which lead to demyelinization, of multiple sclerosis, of infection disorders and viral disorders, for example encephalitis, ischemic stroke, and as medicaments for chemotherapy of cancer, for the treatment of Guillain-Barré syndrome and for the treatment of osteoporosis.

The inventive compounds of the formula I may also find use as medicaments for the treatment of polycystic ovary syndrome (PCOS).

According to the present invention, the compounds of the formula I are particularly useful for the treatment of psychotic complaints, especially of schizophrenia, reduced alertness and hyperactivity (ADHD) in hyperkinetic children, for the treatment of eating disorders and of obesity, for the treatment of type II diabetes, for the treatment of deficits of memory and cognitive deficits, for the treatment of alcohol addiction, of nicotine addiction, i.e. for alcohol and tobacco withdrawal.

The inventive compounds of the formula I are very particularly useful for the treatment and prevention of eating disorders, appetite disorders, metabolic disorders, gastrointestinal disorders, inflammation symptoms, disorders of the immune system, psychotic disorders, alcohol addiction and nicotine addiction.

According to one of its aspects, the invention relates to the use of a compound of the formula I, the pharmaceutically acceptable salts thereof and the solvates or hydrates thereof for the treatment of the above-specified disorders and diseases.

The compound(s) of the formula I may also be administered in combination with further active ingredients.

The amount of a compound of the formula I which is required in order to achieve the desired biological effect is dependent upon a series of factors, for example the specific compound selected, the intended use, the mode of administration and the clinical condition of the patient. The daily dose is generally in the range from 0.3 mg to 100 mg (typically from 3 mg to 50 mg) per day per kilogram of bodyweight, for example 3-10 mg/kg/day. An intravenous dose may, for example, be in the range from 0.3 mg to 1.0 mg/kg and may suitably be administered as an infusion of from 10 ng to 100 ng per kilogram per minute. Suitable infusion solutions for these purposes may, for example, contain from 0.1 ng to 10 mg, typically from 1 ng to 10 mg, per milliliter. Single doses may contain, for example, from 1 mg to 10 g of the active ingredient. Ampoules for injections may therefore contain, for example, from 1 mg to 100 mg, and single dose formulations which can be administered orally, for example tablets or capsules, may contain, for example, from 1.0 to 1000 mg, typically from 10 to 600 mg. The compounds of the formula I may be used for therapy of the above-mentioned conditions as the compounds themselves, although they are preferably in the form of a pharmaceutical composition with an acceptable carrier. The carrier of course has to be acceptable, in the sense that it is compatible with the other constituents of the composition and is not damaging to the health of the patient. The carrier may be a solid or a liquid or both and is preferably formulated with the compound as a single dose, for example as a tablet, which may contain from 0.05 to 95% by weight of the active ingredient. Further pharmaceutically active substances may likewise be present, including further compounds of the formula I. The inventive pharmaceutical compositions may be produced by one of the known pharmaceutical methods which consist essentially in mixing the constituents with pharmacologically acceptable carriers and/or excipients.

Inventive pharmaceutical compositions are those which are suitable for oral, rectal, topical, peroral (for example sublingual) and parenteral (for example subcutaneous, intramuscular, intradermal or intravenous) administration, although the most suitable mode of administration depends in each individual case on the nature and severity of the condition to be treated and on the type of the compound of the formula I used in each case. Coated formulations and coated slow-release formulations are also encompassed by the scope of the invention. Preference is given to acid- and gastric fluid-resistant formulations. Suitable gastric fluid-resistant coatings include cellulose acetate phthalate, polyvinyl acetate phthalate, hydroxypropylmethylcellulose phthalate and anionic polymers of methacrylic acid and methyl methacrylate.

Suitable pharmaceutical preparations for oral administration may be in the form of separate units, for example capsules, cachets, lozenges or tablets, each of which contains a certain amount of the compound of the formula I; as powder or granules; as solution or suspension in an aqueous or nonaqueous liquid; or as an oil-in-water or water-in-oil emulsion. These compositions may, as already mentioned, be prepared by any suitable pharmaceutical method which includes a step in which the active ingredient and the carrier (which may consist of one or more additional constituents) are brought into contact. In general, the compositions are prepared by uniform and homogeneous mixing of the active ingredient with a liquid carrier and/or finely divided solid carrier, after which the product is shaped if necessary. For example, a tablet can thus be produced by compressing or shaping a powder or granules of the compound, optionally with one or more additional constituents. Compressed tablets can be prepared by tableting the compound in free-flowing form, for example a powder or granules, optionally mixed with a binder, lubricant, inert diluent and/or one (or more) surfactants/dispersants in a suitable machine. Shaped tablets can be prepared by shaping the pulverulent compound moistened with an inert liquid diluent in a suitable machine.

Pharmaceutical compositions which are suitable for peroral (sublingual) administration include lozenges which contain a compound of the formula I with a flavoring, customarily sucrose, and gum arabic or tragacanth, and pastilles which include the compound in an inert base, such as gelatin and glycerol or sucrose and gum arabic.

Suitable pharmaceutical compositions for parenteral administration include preferably sterile aqueous preparations of a compound of the formula I which are preferably isotonic with the blood of the intended recipient. These preparations are preferably administered intravenously, although the administration may also be subcutaneous, intramuscular or intradermal as an injection. These preparations can preferably be produced by mixing the compound with water and making the solution obtained sterile and isotonic with the blood. Injectable compositions according to the invention generally contain from 0.1 to 5% by weight of the active compound.

Suitable pharmaceutical compositions for rectal administration are preferably in the form of single dose suppositories. These can be prepared by mixing a compound of the formula I with one or more conventional solid carriers, for example cocoa butter, and shaping the resulting mixture.

Suitable pharmaceutical compositions for topical application on the skin are preferably in the form of an ointment, cream, lotion, paste, spray, aerosol or oil. Useful carriers include petroleum jelly, lanolin, polyethylene glycols, alcohols and combinations of two or more of these substances. The active ingredient is generally present in a concentration of from 0.1 to 15% by weight of the composition, preferably from 0.5 to 2%.

Transdermal administration is also possible. Suitable pharmaceutical compositions for transdermal applications may be in the form of single plasters which are suitable for long-term close contact with the epidermis of the patient. Such plasters suitably contain the active ingredient in an optionally buffered aqueous solution, dissolved and/or dispersed in an adhesive or dispersed in a polymer. A suitable active ingredient concentration is from approx. 1% to 35%, preferably from approx. 3% to 15%. A particular means of releasing the active ingredient may be by electrotransport or iontophoresis, as described, for example, in Pharmaceutical Research, 2(6): 318 (1986).

Further suitable active ingredients for the combination preparations are:

All antidiabetics which are mentioned in the Rote Liste 2007, chapter 12; all weight-reducing agents/appetite suppressants which are mentioned in the Rote Liste 2007, chapter 1; all diuretics which are mentioned in the Rote Liste 2007, chapter 36; all lipid-lowering agents which are mentioned in the Rote Liste 2007, chapter 58. They can be combined with the compound of the invention of the formula I in particular for a synergistic improvement in action. The active ingredient combination can be administered either by separate administration of the active ingredients to the patient or in the form of combination products in which a plurality of active ingredients are present in one pharmaceutical preparation. If the active ingredients are administered separately, this can be done simultaneously or successively. Most of the active ingredients mentioned hereinafter are disclosed in the USP Dictionary of USAN and International Drug Names, US Pharmacopeia, Rockville 2006.

Antidiabetics include insulin and insulin derivatives, for example Lantus® (see www.lantus.com) or HMR 1964 or Levemir® (insulin detemir), Humalog® (Insulin Lispro), Humulin®, VIAject™, SuliXen® or those as described in WO2005005477 (Novo Nordisk), fast-acting insulins (see U.S. Pat. No. 6,221,633), inhalable insulins, for example Exubera®, Nasulin™, or oral insulins, for example IN-105 (Nobex) or Oral-lyn™ (Generex Biotechnology), or Technosphere® Insulin (MannKind) or Cobalamin™ oral insulin, or insulins as described in WO2007128815, WO2007128817, WO2008034881, WO2008049711, or insulins which can be administered transdermally;

GLP-1 derivatives and GLP-1 agonists, for example exenatide or specific formulations thereof, as described, for example, in WO2008061355, liraglutide, taspoglutide (R-1583), albiglutide, lixisenatide or those which have been disclosed in WO 98/08871, WO2005027978, WO2006037811, WO2006037810 by Novo Nordisk A/S, in WO 01/04156 by Zealand or in WO 00/34331 by Beaufour-Ipsen, pramlintide acetate (Symlin; Amylin Pharmaceuticals), AVE-0010, BIM-51077 (R-1583, ITM-077), PC-DAC:Exendin-4 (an exendin-4 analog which is bonded covalently to recombinant human albumin), CVX-73, CVX-98 and CVx-96 (GLP-1 analog which is bonded covalently to a monoclonal antibody which has specific binding sites for the GLP-1 peptide), CNTO-736 (a GLP-1 analog which is bonded to a domain which includes the Fc portion of an antibody), PGC-GLP-1 (GLP-1 bonded to a nanocarrier), agonists, as described, for example, in D. Chen et al., Proc. Natl. Acad. Sci. USA 104 (2007) 943, those as described in WO2006124529, WO2007124461, WO2008062457, WO2008082274, WO2008101017, WO2008081418, WO2008112939, WO2008112941, WO2008113601, WO2008116294, WO2008116648, WO2008119238, peptides, for example obinepitide (TM-30338), amylin receptor agonists, as described, for example, in WO2007104789, analogs of the human GLP-1, as described in WO2007120899, WO2008022015, WO2008056726, and orally active hypoglycemic ingredients.

Antidiabetics also include agonists of the glucose-dependent insulinotropic polypeptide (GIP) receptor, as described, for example, in WO2006121860.

Antidiabetics also include the glucose-dependent insulinotropic polypeptide (GIP), and also analogous compounds, as described, for example, in WO2008021560.

Antidiabetics also include analogs and derivatives of fibroblast growth factor 21 (FGF-21).

The orally active hypoglycemic ingredients preferably include

sulfonylureas,
biguanidines,
meglitinides,
oxadiazolidinediones,
thiazolidinediones,
PPAR and RXR modulators,
glucosidase inhibitors,
inhibitors of glycogen phosphorylase,
glucagon receptor antagonists,
glucokinase activators,
inhibitors of fructose 1,6-bisphosphatase
modulators of glucose transporter 4 (GLUT4),
inhibitors of glutamine-fructose-6-phosphate amidotransferase (GFAT),
GLP-1 agonists,
potassium channel openers, for example pinacidil, cromakalim, diazoxide, or those as described in R. D. Carr et al., Diabetes 52, 2003, 2513.2518, in J. B. Hansen et al, Current Medicinal Chemistry 11, 2004, 1595-1615, in T. M. Tagmose et al., J. Med. Chem. 47, 2004, 3202-3211 or in M. J. Coghlan et al., J. Med. Chem. 44, 2001, 1627-1653, or those which have been disclosed in WO 97/26265 and WO 99/03861 by Novo Nordisk A/S,
active ingredients which act on the ATP-dependent potassium channel of the beta cells,
inhibitors of dipeptidylpeptidase IV (DPP-IV),
insulin sensitizers,
inhibitors of liver enzymes involved in stimulating gluconeogenesis and/or glycogenolysis,
modulators of glucose uptake, of glucose transport and of glucose reabsorption,
modulators of sodium-dependent glucose transporter 1 or 2 (SGLT1, SGLT2),
inhibitors of 11-beta-hydroxysteroid dehydrogenase-1 (11β-HSD1),
inhibitors of protein tyrosine phosphatase 1B (PTP-1B),
nicotinic acid receptor agonists,
inhibitors of hormone-sensitive or endothelial lipases,
inhibitors of acetyl-CoA carboxylase (ACC1 and/or ACC2) or
inhibitors of GSK-3 beta.
Also included are compounds which modify the metabolism, such as active antihyperlipidemic ingredients and active antilipidemic ingredients, HMGCoA reductase inhibitors,
farnesoid X receptor (FXR) modulators,
fibrates,
cholesterol reabsorption inhibitors,
CETP inhibitors,
bile acid reabsorption inhibitors,
MTP inhibitors,
agonists of estrogen receptor gamma (ERRγ agonists),
sigma-1 receptor antagonists,
antagonists of the somatostatin 5 receptor (SST5 receptor);
compounds which reduce food intake, and
compounds which increase thermogenesis.

In one embodiment of the invention, the compound of the formula I is administered in combination with insulin.

In one embodiment, the compound of the formula I is administered in combination with an active ingredient which acts on the ATP-dependent potassium channel of the beta cells, for example sulfonylureas, for example tolbutamide, glibenclamide, glipizide, gliclazide or glimepiride.

In one embodiment, the compound of the formula I is administered in combination with a tablet which comprises both glimepiride, which is released rapidly, and metformin, which is released over a longer period (as described, for example, in US2007264331, WO2008050987, WO2008062273).

In one embodiment, the compound of the formula I is administered in combination with a biguanide, for example metformin.

In another embodiment, the compound of the formula I is administered in combination with a meglitinide, for example repaglinide, nateglinid or mitiglinide.

In a further embodiment, the compound of the formula I is administered with a combination of mitiglinide with a glitazone, e.g. pioglitazone hydrochloride.

In a further embodiment, the compound of the formula I is administered with a combination of mitiglinide with an alpha-glucosidase inhibitor.

In a further embodiment, the compound of the formula I is administered in combination with antidiabetic compounds, as described in WO2007095462, WO2007101060, WO2007105650.

In a further embodiment, the compound of the formula I is administered in combination with antihypoglycemic compounds, as described in WO2007137008, WO2008020607.

In one embodiment, the compound of the formula I is administered in combination with a thiazolidinedione, for example troglitazone, ciglitazone, pioglitazone, rosiglitazone or the compounds disclosed in WO 97/41097 by Dr. Reddy's Research Foundation, especially 5-[[4-[(3,4-dihydro-3-methyl-4-oxo-2-quinazolinylmethoxy]phenyl]methyl]-2,4-thiazolidinedione.

In one embodiment of the invention, the compound of the formula I is administered in combination with a PPAR gamma agonist, for example rosiglitazone, pioglitazone, JTT-501, G1 262570, R-483, CS-011 (rivoglitazone), DRL-17564, DRF-2593 (balaglitazone), INT-131, T-2384, or those as described in WO2005086904, WO2007060992, WO2007100027, WO2007103252, WO2007122970, WO2007138485, WO2008006319, WO2008006969, WO2008010238, WO2008017398, WO2008028188, WO2008066356, WO2008084303, WO2008089461-WO2008089464, WO2008093639, WO2008096769, WO2008096820, WO2008096829, US2008194617, WO2008099944, WO2008108602, WO2008109334, WO2008126731, WO2008126732.

In one embodiment of the invention, the compound of the formula I is administered in combination with Competact™, a solid combination of pioglitazone hydrochloride with metformin hydrochloride.

In one embodiment of the invention, the compound of the formula I is administered in combination with Tandemact™, a solid combination of pioglitazone with glimepiride.

In a further embodiment of the invention, the compound of the formula I is administered in combination with a solid combination of pioglitazone hydrochloride with an angiotensin II agonist, for example TAK-536.

In one embodiment of the invention, the compound of the formula I is administered in combination with a PPAR alpha agonist or mixed PPAR alpha/PPAR delta agonist, for example GW9578, GW-590735, K-111, LY-674, KRP-101, DRF-10945, LY-518674, CP-900691, BMS-687453, BMS-711939, or those as described in WO2001040207, WO2002096894, WO2005097076, WO2007056771, WO2007087448, WO2007089667, WO2007089557, WO2007102515, WO2007103252, JP2007246474, WO2007118963, WO2007118964, WO2007126043, WO2008006043, WO2008006044, WO2008012470, WO2008035359, WO2008087365, WO2008087366, WO2008087367, WO2008117982.

In one embodiment of the invention, the compound of the formula I is administered in combination with a mixed PPAR alpha/gamma agonist, for example naveglitazar, LY-510929, ONO-5129, E-3030, AVE 8042, AVE 8134, AVE 0847, CKD-501 (lobeglitazone sulfate), MBX-213, KY-201 or as described in WO 00/64888, WO 00/64876, WO03/020269, WO2004024726, WO2007099553, US2007276041, WO2007085135, WO2007085136, WO2007141423, WO2008016175, WO2008053331, WO2008109697, WO2008109700, WO2008108735 or in J. P. Berger et al., TRENDS in Pharmacological Sciences 28(5), 244-251, 2005.

In one embodiment of the invention, the compound of the formula I is administered in combination with a PPAR delta agonist, for example GW-501516, or as described in WO2006059744, WO2006084176, WO2006029699, WO2007039172-WO2007039178, WO2007071766, WO2007101864, US2007244094, WO2007119887, WO2007141423, US2008004281, WO2008016175, WO2008066356, WO2008071311, WO2008084962, US2008176861.

In one embodiment of the invention, the compound of the formula I is administered in combination with a pan-SPPARM (selective PPAR modulator alpha, gamma, delta), for example GFT-505, or those as described in WO2008035359.

In one embodiment, the compound of the formula I is administered in combination with metaglidasen or with MBX-2044 or other partial PPAR gamma agonists/antagonists.

In one embodiment, the compound of the formula I is administered in combination with an α-glucosidase inhibitor, for example miglitol or acarbose, or those as described, for example, in WO2007114532, WO2007140230, US2007287674, US2008103201, WO2008065796, WO2008082017.

In one embodiment, the compound of the formula I is administered in combination with an inhibitor of glycogen phosphorylase, for example PSN-357 or FR-258900, or those as described in WO2003084922, WO2004007455, WO2005073229-31, WO2005067932, WO2008062739, WO2008099000, WO2008113760.

In one embodiment, the compound of the formula I is administered in combination with glucagon receptor antagonists, for example A-770077 or NNC-25-2504 or as described in WO2004100875, WO2005065680, WO2006086488, WO2007047177, WO2007106181, WO2007111864, WO2007120270, WO2007120284, WO2007123581, WO2007136577, WO2008042223, WO2008098244.

In a further embodiment, the compound of the formula I is administered in combination with an antisense compound, e.g. ISIS-325568, which inhibits the production of the glucagon receptor.

In one embodiment, the compound of the formula I is administered in combination with activators of glucokinase, for example LY-2121260 (WO2004063179), PSN-105, PSN-110, GKA-50, or those as described, for example, in WO2004072031, WO2004072066, WO2005080360, WO2005044801, WO2006016194, WO2006058923, WO2006112549, WO2006125972, WO2007017549, WO2007017649, WO2007007910, WO2007007040-42, WO2007006760-61, WO2007006814, WO2007007886, WO2007028135, WO2007031739, WO2007041365, WO2007041366, WO2007037534, WO2007043638, WO2007053345, WO2007051846, WO2007051845, WO2007053765, WO2007051847, WO2007061923, WO2007075847, WO2007089512, WO2007104034, WO2007117381, WO2007122482, WO2007125103, WO2007125105, US2007281942, WO2008005914, WO2008005964, WO2008043701, WO2008044777, WO2008047821, US2008096877, WO2008050117, WO2008050101, WO2008059625, US2008146625, WO2008078674, WO2008079787, WO2008084043, WO2008084044, WO2008084872, WO2008089892, WO2008091770, WO2008075073, WO2008084043, WO2008084044, WO2008084872, WO2008084873, WO2008089892, WO2008091770, JP2008189659, WO2008104994, WO2008111473, WO2008116107, WO2008118718, WO2008120754.

In one embodiment, the compound of the formula I is administered in combination with an inhibitor of gluconeogenesis, as described, for example, in FR-225654, WO2008053446.

In one embodiment, the compound of the formula I is administered in combination with inhibitors of fructose 1,6-bisphosphatase (FBPase), for example MB-07729, CS-917 (MB-06322) or MB-07803, or those as described in WO2006023515, WO2006104030, WO2007014619, WO2007137962, WO2008019309, WO2008037628.

In one embodiment, the compound of the formula I is administered in combination with modulators of glucose transporter 4 (GLUT4), for example KST-48 (D.-O. Lee et al.: Arzneim.-Forsch. Drug Res. 54 (12), 835 (2004)).

In one embodiment, the compound of the formula I is administered in combination with inhibitors of glutamine:fructose-6-phosphate amidotransferase (GFAT), as described, for example, in WO2004101528.

In one embodiment, the compound of the formula I is administered in combination with inhibitors of dipeptidyl peptidase IV (DPP-IV), for example vildagliptin (LAF-237), sitagliptin (MK-0431), sitagliptin phosphate, saxagliptin ((BMS-477118), GSK-823093, PSN-9301, SYR-322, SYR-619, TA-6666, TS-021, GRC-8200 (Melogliptin), GW-825964X, KRP-104, DP-893, ABT-341, ABT-279 or another salt thereof, S-40010, S-40755, PF-00734200, BI-1356, PHX-1149, alogliptin benzoate, linagliptin, melogliptin, or those compounds as described in WO2003074500, WO2003106456, WO2004037169, WO200450658, WO2005037828, WO2005058901, WO2005012312, WO2005/012308, WO2006039325, WO2006058064, WO2006015691, WO2006015701, WO2006015699, WO2006015700, WO2006018117, WO2006099943, WO2006099941, JP2006160733, WO2006071752, WO2006065826, WO2006078676, WO2006073167, WO2006068163, WO2006085685, WO2006090915, WO2006104356, WO2006127530, WO2006111261, US2006890898, US2006803357, US2006303661, WO2007015767 (LY-2463665), WO2007024993, WO2007029086, WO2007063928, WO2007070434, WO2007071738, WO2007071576, WO2007077508, WO2007087231, WO2007097931, WO2007099385, WO2007100374, WO2007112347, WO2007112669, WO2007113226, WO2007113634, WO2007115821, WO2007116092, US2007259900, EP1852108, US2007270492, WO2007126745, WO2007136603, WO2007142253, WO2007148185, WO2008017670, US2008051452, WO2008027273, WO2008028662, WO2008029217, JP2008031064, JP2008063256, WO2008033851, WO2008040974, WO2008040995, WO2008060488, WO2008064107, WO2008066070, WO2008077597, JP2008156318, WO2008087560, WO2008089636, WO2008093960, WO2008096841, WO2008101953, WO2008118848, WO2008119005, WO2008119208, WO2008120813, WO2008121506.

In one embodiment, the compound of the formula I is administered in combination with Janumet™, a solid combination of sitagliptin phosphate with metformin hydrochloride.

In one embodiment, the compound of the formula I is administered in combination with Eucreas®, a solid combination of vildagliptin with metformin hydrochloride.

In a further embodiment, the compound of the formula I is administered in combination with a solid combination of alogliptin benzoate with pioglitazone.

In one embodiment, the compound of the formula I is administered in combination with a solid combination of a salt of sitagliptin with metformin hydrochloride.

In one embodiment, the compound of the formula I is administered in combination with a combination of a DPP-IV inhibitor with omega-3 fatty acids or omega-3 fatty acid esters, as described, for example, in WO2007128801.

In one embodiment, the compound of the formula I is administered in combination with a solid combination of a salt of sitagliptin with metformin hydrochloride.

In one embodiment, the compound of the formula I is administered in combination with a substance which enhances insulin secretion, for example KCP-265 (WO2003097064), or those as described in WO2007026761, WO2008045484, US2008194617.

In one embodiment, the compound of the formula I is administered in combination with agonists of the glucose-dependent insulinotropic receptor (GDIR), for example APD-668.

In one embodiment of the invention, the compound of the formula I is administered in combination with an ATP citrate lyase inhibitor, for example SB-204990.

In one embodiment, the compound of the formula I is administered in combination with modulators of the sodium-dependent glucose transporter 1 or 2 (SGLT1, SGLT2), for example KGA-2727, T-1095, SGL-0010, AVE 2268, SAR 7226, SGL-5083, SGL-5085, SGL-5094, ISIS-388626, sergliflozin or dapagliflozin, or as described, for example, in WO2004007517, WO200452903, WO200452902, PCT/EP2005/005959, WO2005085237, JP2004359630, WO2005121161, WO2006018150, WO2006035796, WO2006062224, WO2006058597, WO2006073197, WO2006080577, WO2006087997, WO2006108842, WO2007000445, WO2007014895, WO2007080170, WO2007093610, WO2007126117, WO2007128480, WO2007129668, US2007275907, WO2007136116, WO2007143316, WO2007147478, WO2008001864, WO2008002824, WO2008013277, WO2008013280, WO2008013321, WO2008013322, WO2008016132, WO2008020011, JP2008031161, WO2008034859, WO2008042688, WO2008044762, WO2008046497, WO2008049923, WO2008055870, WO2008055940, WO2008069327, WO2008070609, WO2008071288, WO2008072726, WO2008083200, WO2008090209, WO2008090210, WO2008101586, WO2008101939, WO2008116179, WO2008116195, US2008242596 or by A. L. Handlon in Expert Opin. Ther. Patents (2005) 15(11), 1531-1540.

In one embodiment, the compound of the formula I is administered in combination with inhibitors of 1′-beta-hydroxysteroid dehydrogenase 1 (11β-HSD1), for example BVT-2733, JNJ-25918646, INCB-13739, INCB-20817, DIO-92 ((−)-ketoconazole) or those as described, for example, in WO200190090-94, WO200343999, WO2004112782, WO200344000, WO200344009, WO2004112779, WO2004113310, WO2004103980, WO2004112784, WO2003065983, WO2003104207, WO2003104208, WO2004106294, WO2004011410, WO2004033427, WO2004041264, WO2004037251, WO2004056744, WO2004058730, WO2004065351, WO2004089367, WO2004089380, WO2004089470-71, WO2004089896, WO2005016877, WO2005063247, WO2005097759, WO2006010546, WO2006012227, WO2006012173, WO2006017542, WO2006034804, WO2006040329, WO2006051662, WO2006048750, WO2006049952, WO2006048331, WO2006050908, WO2006024627, WO2006040329, WO2006066109, WO2006074244, WO2006078006, WO2006106423, WO2006132436, WO2006134481, WO2006134467, WO2006135795, WO2006136502, WO2006138508, WO2006138695, WO2006133926, WO2007003521, WO2007007688, US2007066584, WO2007029021, WO2007047625, WO2007051811, WO2007051810, WO2007057768, WO2007058346, WO2007061661, WO2007068330, WO2007070506, WO2007087150, WO2007092435, WO2007089683, WO2007101270, WO2007105753, WO2007107470, WO2007107550, WO2007111921, US2007207985, US2007208001, WO2007115935, WO2007118185, WO2007122411, WO2007124329, WO2007124337, WO2007124254, WO2007127688, WO2007127693, WO2007127704, WO2007127726, WO2007127763, WO2007127765, WO2007127901, US2007270424, JP2007291075, WO2007130898, WO2007135427, WO2007139992, WO2007144394, WO2007145834, WO2007145835, WO2007146761, WO2008000950, WO2008000951, WO2008003611, WO2008005910, WO2008006702, WO2008006703, WO2008011453, WO2008012532, WO2008024497, WO2008024892, WO2008032164, WO2008034032, WO2008043544, WO2008044656, WO2008046758, WO2008052638, WO2008053194, WO2008071169, WO2008074384, WO2008076336, WO2008076862, WO2008078725, WO2008087654, WO2008088540, WO2008099145, WO2008101885, WO2008101886, WO2008101907, WO2008101914, WO2008106128, WO2008110196, WO2008119017, WO2008120655, WO2008127924.

In one embodiment, the compound of the formula I is administered in combination with inhibitors of protein tyrosine phosphatase 1B (PTP-1B), as described, for example, in WO200119830-31, WO200117516, WO2004506446, WO2005012295, WO2005116003, WO2005116003, WO2006007959, DE 10 2004 060542.4, WO2007009911, WO2007028145, WO2007067612-615, WO2007081755, WO2007115058, US2008004325, WO2008033455, WO2008033931, WO2008033932, WO2008033934, WO2008089581.

In one embodiment of the invention, the compound of the formula I is administered in combination with an agonist of GPR109A (HM74A receptor agonists; NAR agonists (nicotinic acid receptor agonists)), for example nicotinic acid or “extended release niacin” in conjunction with MK-0524A (laropiprant) or MK-0524, or those compounds as described in WO2004041274, WO2006045565, WO2006045564, WO2006069242, WO2006085108, WO2006085112, WO2006085113, WO2006124490, WO2006113150, WO2007017261, WO2007017262, WO2007017265, WO2007015744, WO2007027532, WO2007092364, WO2007120575, WO2007134986, WO2007150025, WO2007150026, WO2008016968, WO2008051403, WO2008086949, WO2008091338, WO2008097535, WO2008099448, US2008234277, WO2008127591.

In another embodiment of the invention, the compound of the formula I is administered in combination with a solid combination of niacin with simvastatin.

In another embodiment of the invention, the compound of the formula I is administered in combination with nicotinic acid or “extended release niacin” in conjunction with MK-0524A (laropiprant).

In a further embodiment of the invention, the compound of the formula I is administered in combination with nicotinic acid or “extended release niacin” in conjunction with MK-0524A (laropiprant) and with simvastatin.

In one embodiment of the invention, the compound of the formula I is administered in combination with nicotinic acid or another nicotinic acid receptor agonist and a prostaglandin DP receptor antagonist, for example those as described in WO2008039882.

In another embodiment of the invention, the compound of the formula I is administered in combination with an agonist of GPR116, as described, for example, in WO2006067531, WO2006067532.

In one embodiment, the compound of the formula I is administered in combination with modulators of GPR40, as described, for example, in WO2007013689, WO2007033002, WO2007106469, US2007265332, WO2007123225, WO2007131619, WO2007131620, WO2007131621, US2007265332, WO2007131622, WO2007136572, WO2008001931, WO2008030520, WO2008030618, WO2008054674, WO2008054675, WO2008066097, US2008176912.

In one embodiment, the compound of the formula I is administered in combination with modulators of GPR119 (G-protein-coupled glucose-dependent insulinotropic receptor), for example PSN-119-1, PSN-821, PSN-119-2, MBX-2982 or those as described, for example, in WO2004065380, WO2005061489 (PSN-632408), WO2006083491, WO2007003960-62 and WO2007003964, WO2007035355, WO2007116229, WO2007116230, WO2008005569, WO2008005576, WO2008008887, WO2008008895, WO2008025798, WO2008025799, WO2008025800, WO2008070692, WO2008076243, WO200807692, WO2008081204, WO2008081205, WO2008081206, WO2008081207, WO2008081208, WO2008083238, WO2008085316, WO2008109702.

In a further embodiment, the compound of the formula I is administered in combination with modulators of GPR120, as described, for example, in EP1688138, WO2008066131, WO2008066131, WO2008103500, WO2008103501.

In one embodiment, the compound of the formula I is administered in combination with inhibitors of hormone-sensitive lipase (HSL) and/or phospholipases, as described, for example, in WO2005073199, WO2006074957, WO2006087309, WO2006111321, WO2007042178, WO2007119837, WO2008122352, WO2008122357.

In one embodiment, the compound of the formula I is administered in combination with inhibitors of endothelial lipase, as described, for example, in WO2007110216.

In one embodiment, the compound of the formula I is administered in combination with a phospholipase A2 inhibitor, for example darapladib or A-002, or those as described in WO2008048866, WO20080488867.

In one embodiment, the compound of the formula I is administered in combination with myricitrin, a lipase inhibitor (WO2007119827).

In one embodiment, the compound of the formula I is administered in combination with an inhibitor of glycogen synthase kinase-3 beta (GSK-3 beta), as described, for example, in US2005222220, WO2005085230, WO2005111018, WO2003078403, WO2004022544, WO2003106410, WO2005058908, US2005038023, WO2005009997, US2005026984, WO2005000836, WO2004106343, EP1460075, WO2004014910, WO2003076442, WO2005087727, WO2004046117, WO2007073117, WO2007083978, WO2007120102, WO2007122634, WO2007125109, WO2007125110, US2007281949, WO2008002244, WO2008002245, WO2008016123, WO2008023239, WO2008044700, WO2008056266, WO2008057940, WO2008077138, EP1939191, EP1939192, WO2008078196, WO2008094992, WO2008112642, WO2008112651, WO2008113469, WO2008121063, WO2008121064.

In one embodiment, the compound of the formula I is administered in combination with an inhibitor of phosphoenolpyruvate carboxykinase (PEPCK), for example those as described in WO2004074288.

In one embodiment, the compound of the formula I is administered in combination with an inhibitor of phosphoinositide kinase-3 (PI3K), for example those as described in WO2008027584, WO2008070150, WO2008125833, WO2008125835, WO2008125839.

In one embodiment, the compound of the formula I is administered in combination with an inhibitor of serum/glucocorticoid-regulated kinase (SGK), as described, for example, in WO2006072354, WO2007093264, WO2008009335, WO2008086854.

In one embodiment, the compound of the formula I is administered in combination with a modulator of the glucocorticoid receptor, as described, for example, in WO2008057855, WO2008057856, WO2008057857, WO2008057859, WO2008057862, WO2008059867, WO2008059866, WO2008059865, WO2008070507, WO2008124665, WO2008124745.

In one embodiment, the compound of the formula I is administered in combination with a modulator of the mineralocorticoid receptor (MR), for example drospirenone, or those as described in WO2008104306, WO2008119918.

In one embodiment, the compound of the formula I is administered in combination with an inhibitor of protein kinase C beta (PKC beta), for example ruboxistaurin, or those as described in WO2008096260, WO2008125945.

In one embodiment, the compound of the formula I is administered in combination with an inhibitor of protein kinase D, for example doxazosin (WO2008088006).

In a further embodiment, the compound of the formula I is administered in combination with an activator of the AMP-activated protein kinase (AMPK), as described, for example, in WO2007062568, WO2008006432, WO2008016278, WO2008016730, WO2008083124.

In one embodiment, the compound of the formula I is administered in combination with an inhibitor of ceramide kinase, as described, for example, in WO2007112914, WO2007149865.

In a further embodiment, the compound of the formula I is administered in combination with an inhibitor of MAPK-interacting kinase 1 or 2 (MNK1 or 2), as described, for example, in WO2007104053, WO2007115822, WO2008008547, WO2008075741.

In one embodiment, the compound of the formula I is administered in combination with inhibitors of “I-kappaB kinase” (IKK inhibitors), as described, for example, in WO2001000610, WO2001030774, WO2004022057, WO2004022553, WO2005097129, WO2005113544, US2007244140, WO2008099072, WO2008099073, WO2008099073, WO2008099074, WO2008099075.

In another embodiment, the compound of the formula I is administered in combination with inhibitors of NF-kappaB (NFKB) activation, for example salsalate.

In a further embodiment, the compound of the formula I is administered in combination with inhibitors of ASK-1 (apoptosis signal-regulating kinase 1), as described, for example, in WO2008016131.

In one embodiment of the invention, the compound of the formula I is administered in combination with an HMG-CoA reductase inhibitor such as simvastatin, fluvastatin, pravastatin, lovastatin, atorvastatin, cerivastatin, rosuvastatin, pitavastatin, L-659699, BMS-644950, or those as described in US2007249583, WO2008083551.

In a further embodiment of the invention, the compound of the formula I is administered in combination with a farnesoid X receptor (FXR) modulator, for example WAY-362450 or those as described in WO2003099821, WO2005056554, WO2007052843, WO2007070796, WO2007092751, JP2007230909, WO2007095174, WO2007140174, WO2007140183, WO2008000643, WO2008002573, WO2008025539, WO2008025540, JP2008214222.

In another embodiment of the invention, the compound of the formula I is administered in combination with a ligand of the liver X receptor (LXR), as described, for example, in WO2007092965, WO2008041003, WO2008049047, WO2008065754, WO2008073825, US2008242677.

In one embodiment of the invention, the compound of the formula I is administered in combination with a fibrate, for example fenofibrate, clofibrate, bezafibrate, or those as described in WO2008093655.

In one embodiment of the invention, the compound of the formula I is administered in combination with fibrates, for example the choline salt of fenofibrate (SLV-348).

In one embodiment of the invention, the compound of the formula I is administered in combination with fibrates, for example the choline salt of fenofibrate and an HMG-CoA reductase inhibitor, for example rosuvastatin.

In a further embodiment of the invention, the compound of the formula I is administered in combination with bezafibrate and diflunisal.

In a further embodiment of the invention, the compound of the formula I is administered in combination with a solid combination of fenofibrate or a salt thereof with simvastatin, rosuvastatin, fluvastatin, lovastatin, cerivastatin, pravastatin, pitavastatin or atorvastatin.

In a further embodiment of the invention, the compound of the formula I is administered in combination with Synordia (R), a solid combination of fenofibrate with metformin.

In one embodiment of the invention, the compound of the formula I is administered in combination with a cholesterol reabsorption inhibitor, for example ezetimibe, tiqueside, pamaqueside, FM-VP4 (sitostanol/campesterol ascorbyl phosphate; Forbes Medi-Tech, WO2005042692, WO2005005453), MD-0727 (Microbia Inc., WO2005021497, WO2005021495) or with compounds as described in WO2002066464, WO2005000353 (Kotobuki Pharmaceutical Co. Ltd.) or WO2005044256 or WO2005062824 (Merck & Co.) or WO2005061451 and WO2005061452 (AstraZeneca AB) and WO2006017257 (Phenomix) or WO2005033100 (Lipideon Biotechnology AG), or as described in WO2002050060, WO2002050068, WO2004000803, WO2004000804, WO2004000805, WO2004087655, WO2004097655, WO2005047248, WO2006086562, WO2006102674, WO2006116499, WO2006121861, WO2006122186, WO2006122216, WO2006127893, WO2006137794, WO2006137796, WO2006137782, WO2006137793, WO2006137797, WO2006137795, WO2006137792, WO2006138163, WO2007059871, US2007232688, WO2007126358, WO2008033431, WO2008033465, WO2008052658, WO2008057336, WO2008085300.

In one embodiment of the invention, the compound of the formula I is administered in combination with an NPC1L1 antagonist, for example those as described in WO2008033464, WO2008033465.

In one embodiment of the invention, the compound of the formula I is administered in combination with Vytorin™, a solid combination of ezetimibe with simvastatin.

In one embodiment of the invention, the compound of the formula I is administered in combination with a solid combination of ezetimibe with atorvastatin.

In one embodiment of the invention, the compound of the formula I is administered in combination with a solid combination of ezetimibe with fenofibrate.

In one embodiment of the invention, the further active ingredient is a diphenylazetidinone derivative, as described, for example, in U.S. Pat. No. 6,992,067 or U.S. Pat. No. 7,205,290.

In a further embodiment of the invention, the further active ingredient is a diphenylazetidinone derivative, as described, for example, in U.S. Pat. No. 6,992,067 or U.S. Pat. No. 7,205,290, combined with a statin, for example simvastatin, fluvastatin, pravastatin, lovastatin, cerivastatin, atorvastatin, pitavastatin or rosuvastatin.

In one embodiment of the invention, the compound of the formula I is administered in combination with a solid combination of lapaquistat, a squalene synthase inhibitor, with atorvastatin.

In one embodiment of the invention, the compound of the formula I is administered in combination with a CETP inhibitor, for example torcetrapib, anacetrapib or JTT-705 (dalcetrapib), or those as described in WO2006002342, WO2006010422, WO2006012093, WO2006073973, WO2006072362, WO2007088996, WO2007088999, US2007185058, US2007185113, US2007185154, US2007185182, WO2006097169, WO2007041494, WO2007090752, WO2007107243, WO2007120621, US2007265252, US2007265304, WO2007128568, WO2007132906, WO2008006257, WO2008009435, WO2008018529, WO2008058961, WO2008058967, WO2008059513, WO2008070496, WO2008115442, WO2008111604.

In one embodiment of the invention, the compound of the formula I is administered in combination with bile acid reabsorption inhibitor (inhibitors of the intestinal bile acid transporter (IBAT)) (see, for example, U.S. Pat. No. 6,245,744, U.S. Pat. No. 6,221,897 or WO00/61568), for example HMR 1741, or those as described in DE 10 2005 033099.1 and DE 10 2005 033100.9, DE 10 2006 053635, DE 10 2006 053637, WO2007009655-56, WO2008058628, WO2008058629, WO2008058630, WO2008058631.

In one embodiment, the compound of the formula I is administered in combination with agonists of GPBAR1 (G-protein-coupled bile acid receptor-1; TGR5), as described, for example, in US20060199795, WO2007110237, WO2007127505, WO2008009407, WO2008067219, WO2008067222, FR2908310, WO2008091540, WO2008097976.

In one embodiment of the invention, the compound of the formula I is administered in combination with inhibitors of the TRPM5 channel (TRP cation channel M5), as described, for example, in WO2008097504.

In one embodiment of the invention, the compound of the formula I is administered in combination with a polymeric bile acid adsorber, for example cholestyramine, colesevelam hydrochloride.

In one embodiment of the invention, the compound of the formula I is administered in combination with colesevelam hydrochloride and metformin or a sulfonylurea or insulin.

In one embodiment of the invention, the compound of the formula I is administered in combination with a chewing gum comprising phytosterols (Reductol™).

In one embodiment of the invention, the compound of the formula I is administered in combination with an inhibitor of the microsomal triglyceride transfer protein (MTP inhibitor), for example implitapide, BMS-201038, R-103757, AS-1552133, SLx-4090, AEGR-733, or those as described in WO2005085226, WO2005121091, WO2006010423, WO2006113910, WO2007143164, WO2008049806, WO2008049808, WO2008090198, WO2008100423.

In a further embodiment of the invention, the compound of the formula I is administered in combination with a combination of a cholesterol absorption inhibitor, for example ezetimibe, and an inhibitor of the triglyceride transfer proteins (MTP inhibitor), for example implitapide, as described in WO2008030382 or in WO2008079398.

In one embodiment of the invention, the compound of the formula I is administered in combination with an active antihypertriglyceridemic ingredient, for example those as described in WO2008032980.

In another embodiment of the invention, the compound of the formula I is administered in combination with an antagonist of the somatostatin 5 receptor (SST5 receptor), for example those as described in WO2006094682.

In one embodiment of the invention, the compound of the formula I is administered in combination with an ACAT inhibitor, for example avasimibe, SMP-797 or KY-382, or those as described in WO2008087029, WO2008087030, WO2008095189.

In a further embodiment of the invention, the compound of the formula I is administered in combination with an inhibitor of liver carnitine palmitoyltransferase 1 (L-CPT1), as described, for example, in WO2007063012, WO2007096251 (ST-3473), WO2008015081, US2008103182, WO2008074692.

In a further embodiment of the invention, the compound of the formula I is administered in combination with a modulator of serine palmitoyltransferase (SPT), as described, for example, in WO2008031032, WO2008046071, WO2008083280, WO2008084300.

In one embodiment of the invention, the compound of the formula I is administered in combination with a squalene synthetase inhibitor, for example BMS-188494, TAK-475 (lapaquistat acetate), or as described in WO2005077907, JP2007022943, WO2008003424.

In one embodiment of the invention, the compound of the formula I is administered in combination with ISIS-301012 (mipomersen), an antisense oligonucleotide which is capable of regulating the apolipoprotein B gene.

In one embodiment of the invention, the compound of the formula I is administered in combination with a stimulator of the ApoA-1 gene, as described, for example in WO2008092231.

In one embodiment of the invention, the compound of the formula I is administered in combination with an LDL receptor inducer (see U.S. Pat. No. 6,342,512), for example HMR1171, HMR1586, or those as described in WO2005097738, WO2008020607.

In another embodiment of the invention, the compound of the formula I is administered in combination with an HDL cholesterol-elevating agent, for example those as described in WO2008040651, WO2008099278.

In one embodiment of the invention, the compound of the formula I is administered in combination with an ABCA1 expression enhancer, as described, for example, in WO2006072393, WO2008062830.

In one embodiment of the invention, the compound of the formula I is administered in combination with a lipoprotein lipase modulator, for example ibrolipim (NO-1886).

In one embodiment of the invention, the compound of the formula I is administered in combination with a lipoprotein(a) antagonist, for example gemcabene (CI-1027).

In one embodiment of the invention, the compound of the formula I is administered in combination with a lipase inhibitor, for example orlistat or cetilistat (ATL-962).

In one embodiment of the invention, the compound of the formula I is administered in combination with an adenosine A1 receptor agonist (adenosine A1 R), as described, for example, in EP1258247, EP1375508, WO2008028590, WO2008077050.

In one embodiment of the invention, the compound of the formula I is administered in combination with an adenosine A2B receptor agonist (adenosine A2B R), for example ATL-801.

In another embodiment of the invention, the compound of the formula I is administered in combination with a modulator of adenosine A2A and/or adenosine A3 receptors, as described, for example, in WO2007111954, WO2007121918, WO2007121921, WO2007121923, WO2008070661.

In a further embodiment of the invention, the compound of the formula I is administered in combination with an agonist of the adenosine A1/A2B receptors, as described, for example, in WO2008064788, WO2008064789.

In one embodiment of the invention, the compound of the formula I is administered in combination with an adenosine A2B receptor antagonist (adenosine A2B R), as described in US2007270433, WO2008027585, WO2008080461.

In one embodiment, the compound of the formula I is administered in combination with inhibitors of acetyl-CoA carboxylase (ACC1 and/or ACC2), for example those as described in WO199946262, WO200372197, WO2003072197, WO2005044814, WO2005108370, JP2006131559, WO2007011809, WO2007011811, WO2007013691, WO2007095601-603, WO2007119833, WO2008065508, WO2008069500, WO2008070609, WO2008072850, WO2008079610, WO2008088688, WO2008088689, WO2008088692, US2008171761, WO2008090944, JP2008179621, US2008200461, WO2008102749, WO2008103382, WO2008121592.

In another embodiment, the compound of the formula I is administered in combination with modulators of microsomal acyl-CoA:glycerol-3-phosphate acyltransferase 3 (GPAT3, described in WO2007100789) or with modulators of microsomal acyl-CoA:glycerol-3-phosphate acyltransferase 4 (GPAT4, described in WO2007100833).

In a further embodiment, the compound of the formula I is administered in combination with modulators of xanthine oxidoreductase (XOR).

In another embodiment, the compound of the formula I is administered in combination with inhibitors of soluble epoxide hydrolase (sEH), as described, for example, in WO2008051873, WO2008051875, WO2008073623, WO2008094869, WO2008112022.

In a further embodiment, the compound of the formula I is administered in combination with CART modulators (see “Cocaine-amphetamine-regulated transcript influences energy metabolism, anxiety and gastric emptying in mice” Asakawa, A. et al.: Hormone and Metabolic Research (2001), 33(9), 554-558);

NPY antagonists, for example N-{4-[(4-aminoquinazolin-2-ylamino)methyl]-cyclohexylmethyl}naphthalene-1-sulfonamide hydrochloride (CGP 71683A) or velneperit;
NPY-5 receptor antagonists, such as L-152804 or the compound “NPY-5-BY” from Banyu, or as described, for example, in WO2006001318, WO2007103295, WO2007125952, WO2008026563, WO2008026564, WO2008052769, WO2008092887, WO2008092888, WO2008092891;
NPY-4 receptor antagonists, as described, for example, in WO2007038942;
NPY-2 receptor antagonists, as described, for example, in WO2007038943;
peptide YY 3-36 (PYY3-36) or analogous compounds, for example CJC-1682 (PYY3-36 conjugated with human serum albumin via Cys34) or CJC-1643 (derivative of PYY3-36, which is conjugated in vivo to serum albumin), or those as described in WO2005080424, WO2006095166, WO2008003947;
derivatives of the peptide obestatin, as described by WO2006096847;
CB1R (cannabinoid receptor 1) antagonists, for example rimonabant, surinabant (SR147778), SLV-319 (ibipinabant), AVE-1625, taranabant (MK-0364) or salts thereof, otenabant (CP-945,598), rosonabant, V-24343 or those compounds as described in, for example, EP 0656354, WO 00/15609, WO2001/64632-64634, WO 02/076949, WO2005080345, WO2005080328, WO2005080343, WO2005075450, WO2005080357, WO200170700, WO2003026647-48, WO200302776, WO2003040107, WO2003007887, WO2003027069, U.S. Pat. No. 6,509,367, WO200132663, WO2003086288, WO2003087037, WO2004048317, WO2004058145, WO2003084930, WO2003084943, WO2004058744, WO2004013120, WO2004029204, WO2004035566, WO2004058249, WO2004058255, WO2004058727, WO2004069838, US20040214837, US20040214855, US20040214856, WO2004096209, WO2004096763, WO2004096794, WO2005000809, WO2004099157, US20040266845, WO2004110453, WO2004108728, WO2004000817, WO2005000820, US20050009870, WO200500974, WO2004111033-34, WO200411038-39, WO2005016286, WO2005007111, WO2005007628, US20050054679, WO2005027837, WO2005028456, WO2005063761-62, WO2005061509, WO2005077897, WO2006018662, WO2006047516, WO2006060461, WO2006067428, WO2006067443, WO2006087480, WO2006087476, WO2006100208, WO2006106054, WO2006111849, WO2006113704, WO2007009705, WO2007017124, WO2007017126, WO2007018459, WO2007018460, WO2007016460, WO2007020502, WO2007026215, WO2007028849, WO2007031720, WO2007031721, WO2007036945, WO2007038045, WO2007039740, US20070015810, WO2007046548, WO2007047737, WO2007057687, WO2007062193, WO2007064272, WO2007079681, WO2007084319, WO2007084450, WO2007086080, EP1816125, US2007213302, WO2007095513, WO2007096764, US2007254863, WO2007119001, WO2007120454, WO2007121687, WO2007123949, US2007259934, WO2007131219, WO2007133820, WO2007136571, WO2007136607, WO2007136571, U.S. Pat. No. 7,297,710, WO2007138050, WO2007139464, WO2007140385, WO2007140439, WO2007146761, WO2007148061, WO2007148062, US2007293509, WO2008004698, WO2008017381, US2008021031, WO2008024284, WO2008031734, WO2008032164, WO2008034032, WO2008035356, WO2008036021, WO2008036022, WO2008039023, WO2998043544, WO2008044111, WO2008048648, EP1921072-A1, WO2008053341, WO2008056377, WO2008059207, WO2008059335, WO2008062424, WO2008068423, WO2008068424, WO2008070305, WO2008070306, WO2008074816, WO2008074982, WO2008075012, WO2008075013, WO2008075019, WO2008075118, WO2008076754, WO2008081009, WO2008084057, EP1944295, US2008090809, US2008090810, WO2008092816, WO2008094473, WO2008094476, WO2008099076, WO2008099139, WO2008101995, US2008207704, WO2008107179, WO2008109027, WO2008112674, WO2008115705, WO2008118414, WO2008119999, WO200812000, WO2008121257, WO2008127585;
cannabinoid receptor l/cannabinoid receptor 2 (CB1/CB2) modulating compounds, for example delta-9-tetrahydrocannabivarin, or those as described, for example, in WO2007001939, WO2007044215, WO2007047737, WO2007095513, WO2007096764, WO2007112399, WO2007112402, WO2008122618;
modulators of FAAH (fatty acid amide hydrolase), as described, for example, in WO2007140005, WO2008019357, WO2008021625, WO2008023720, WO2008030532;
inhibitors of fatty acid synthase (FAS), as described, for example, in WO2008057585, WO2008059214, WO2008075064, WO2008075070, WO2008075077;
inhibitors of LCE (long chain fatty acid elongase), as described, for example, in WO2008120653;
vanilloid-1 receptor modulators (modulators of TRPV1), as described, for example, in WO2007091948, WO2007129188, WO2007133637, WO2008007780, WO2008010061, WO2008007211, WO2008010061, WO2008015335, WO2008018827, WO2008024433, WO2008024438, WO2008032204, WO2008050199, WO2008059339, WO2008059370, WO2008066664, WO2008075150, WO2008090382, WO2008090434, WO2008093024, WO2008107543, WO2008107544, WO2008110863;
modulators, antagonists or inverse agonists of the opioid receptors, for example GSK-982 or those as described, for example, in WO2007047397, WO2008021849, WO2008021851, WO2008032156, WO2008059335;
modulators of the “orphan opioid (ORL-1) receptor”, as described, for example, in US2008249122, WO2008089201;
agonists of the prostaglandin receptor, for example bimatoprost or those compounds as described in WO2007111806;
MC4 receptor agonists (melanocortin-4 receptor agonists, MC4R agonists, for example N-[2-(3a-benzyl-2-methyl-3-oxo-2,3,3a,4,6,7-hexahydropyrazolo[4,3-c]-pyridin-5-yl)-1-(4-chlorophenyl)-2-oxoethyl]-1-amino-1,2,3,4-tetrahydronaphthalene-2-carboxamide; (WO 01/91752)) or LB53280, LB53279, LB53278 or THIQ, MB243, RY764, CHIR-785, PT-141, MK-0493, or those as described in WO2005060985, WO2005009950, WO2004087159, WO2004078717, WO2004078716, WO2004024720, US20050124652, WO2005051391, WO2004112793, WOUS20050222014, US20050176728, US20050164914, US20050124636, US20050130988, US20040167201, WO2004005324, WO2004037797, WO2005042516, WO2005040109, WO2005030797, US20040224901, WO200501921, WO200509184, WO2005000339, EP1460069, WO2005047253, WO2005047251, WO2005118573, EP1538159, WO2004072076, WO2004072077, WO2006021655-57, WO2007009894, WO2007015162, WO2007041061, WO2007041052, JP2007131570, EP-1842846, WO2007096186, WO2007096763, WO2007141343, WO2008007930, WO2008017852, WO2008039418, WO2008087186, WO2008087187, WO2008087189, WO2008087186-WO2008087190, WO2008090357;
orexin receptor 1 antagonists (OX1R antagonists), orexin receptor 2 antagonists (OX2R antagonists) or mixed OX1R/OX2R antagonists (e.g. 1-(2-methyl-benzoxazol-6-yl)-3-[1,5]naphthyridin-4-ylurea hydrochloride (SB-334867-A), or those as described, for example, in WO200196302, WO200185693, WO2004085403, WO2005075458, WO2006067224, WO2007085718, WO2007088276, WO2007116374; WO2007122591, WO2007126934, WO2007126935, WO2008008517, WO2008008518, WO2008008551, WO2008020405, WO2008026149, WO2008038251, US2008132490, WO2008065626, WO2008078291, WO2008087611, WO2008081399, WO2008108991, WO2008107335, US2008249125);
histamine H3 receptor antagonists/inverse agonists (e.g. 3-cyclohexyl-1-(4,4-dimethyl-1,4,6,7-tetrahydroimidazo[4,5-c]pyridin-5-yl)propan-1-one oxalic acid salt (WO 00/63208), or those as described in WO200064884, WO2005082893, US2005171181 (e.g. PF-00389027), WO2006107661, WO2007003804, WO2007016496, WO2007020213, WO2007049798, WO2007055418, WO2007057329, WO2007065820, WO2007068620, WO2007068641, WO2007075629, WO2007080140, WO2007082840, WO2007088450, WO2007088462, WO2007094962, WO2007099423, WO2007100990, WO2007105053, WO2007106349, WO2007110364, WO2007115938, WO2007131907, WO2007133561, US2007270440, WO2007135111, WO2007137955, US2007281923, WO2007137968, WO2007138431, WO2007146122, WO2008005338, WO2008012010, WO2008015125, WO2008045371, EP1757594, WO2008068173, WO2008068174, US20080171753, WO2008072703, WO2008072724, US2008188484, US2008188486, US2008188487, WO2008109333, WO2008109336);
histamine H1/histamine H3 modulators, for example betahistine or its dihydrochloride;
modulators of the histamine H3 transporter or of the histamine H3/serotonin transporter, as described, for example, in WO2008002816, WO2008002817, WO2008002818, WO2008002820;
histamine H4 modulators, as described, for example, in WO2007117399;
CRF antagonists (e.g. [2-methyl-9-(2,4,6-trimethylphenyl)-9H-1,3,9-triazafluoren-4-yl]dipropylamine (WO 00/66585) or those CRF1 antagonists as described in WO2007105113, WO2007133756, WO2008036541, WO2008036579, WO2008083070);
CRF BP antagonists (e.g. urocortin);
urocortin agonists;
modulators of the beta-3 adrenoceptor, for example 1-(4-chloro-3-methanesulfonylmethylphenyl)-2-[2-(2,3-dimethyl-1H-indol-6-yloxy)ethylamino]ethanol hydrochloride (WO 01/83451) or solabegron (GW-427353) or N-5984 (KRP-204), or those as described in JP2006111553, WO2002038543, WO2002038544, WO2007048840-843, WO2008015558, EP1947103;
MSH (melanocyte-stimulating hormone) agonists;
MCH (melanine-concentrating hormone) receptor antagonists (for example NBI-845, A-761, A-665798, A-798, ATC-0175, T-226296, T-71 (AMG-071, AMG-076), GW-856464, NGD-4715, ATC-0453, ATC-0759, GW-803430, or those compounds as described in WO2005085200, WO2005019240, WO2004011438, WO2004012648, WO2003015769, WO2004072025, WO2005070898, WO2005070925, WO2004039780, WO2004092181, WO2003033476, WO2002006245, WO2002089729, WO2002002744, WO2003004027, FR2868780, WO2006010446, WO2006038680, WO2006044293, WO2006044174, JP2006176443, WO2006018280, WO2006018279, WO2006118320, WO2006130075, WO2007018248, WO2007012661, WO2007029847, WO2007024004, WO2007039462, WO2007042660, WO2007042668, WO2007042669, US2007093508, US2007093509, WO2007048802, JP2007091649, WO2007092416; WO2007093363-366, WO2007114902, WO2007114916, WO2007141200, WO2007142217, US2007299062, WO2007146758, WO2007146759, WO2008001160, WO2008016811, WO2008020799, WO2008022979, WO2008038692, WO2008041090, WO2008044632, WO2008047544, WO2008061109, WO2008065021, WO2008068265, WO2008071646, WO2008076562, JP2008088120, WO2008086404, WO2008086409);
CCK-A (CCK-1) agonists/modulators (for example {2-[4-(4-chloro-2,5-dimethoxyphenyl)-5-(2-cyclohexylethyl)thiazol-2-ylcarbamoyl]-5,7-dimethylindol-1-yl}acetic acid trifluoroacetic acid salt (WO 99/15525) or SR-146131 (WO 0244150) or SSR-125180), or those as described in WO2005116034, WO2007120655, WO2007120688, WO2007120718, WO2008091631;
serotonin reuptake inhibitors (e.g. dexfenfluramine), or those as described in WO2007148341, WO2008034142, WO2008081477, WO2008120761;
mixed serotonin/dopamine reuptake inhibitors (e.g. bupropion), or those as described in WO2008063673, or solid combinations of bupropion with naltrexone or bupropion with zonisamide;
mixed reuptake inhibitors, for example DOV-21947;
mixed serotoninergic and noradrenergic compounds (e.g. WO 00/71549);
5-HT receptor agonists, for example 1-(3-ethylbenzofuran-7-yl)piperazine oxalic acid salt (WO 01/09111);
mixed dopamine/norepinephrine/acetylcholine reuptake inhibitors (e.g. tesofensine), or those as described, for example, in WO2006085118;
dopamine antagonists, as described, for example, in WO2008079838, WO2008079839, WO2008079847, WO2008079848;
norepinephrine reuptake inhibitors, as described, for example, in US2008076724;
5-HT2A receptor antagonists, as described, for example, in WO2007138343;
5-HT2C receptor agonists (for example lorcaserine hydrochloride (APD-356) or BVT-933, or those as described in WO200077010, WO200077001-02, WO2005019180, WO2003064423, WO200242304, WO2005035533, WO2005082859, WO2006004937, US2006025601, WO2006028961, WO2006077025, WO2006103511, WO2007028132, WO2007084622, US2007249709; WO2007132841, WO2007140213, WO2008007661, WO2008007664, WO2008009125, WO2008010073, WO2008108445);
5-HT6 receptor modulators, for example E-6837, BVT-74316 or PRX-07034, or those as described, for example, in WO2005058858, WO2007054257, WO2007107373, WO2007108569, WO2007108742-744, WO2008003703, WO2008027073, WO2008034815, WO2008054288, EP1947085, WO2008084491, WO2008084492, WO2008092665, WO2008092666, WO2008101247, WO2008110598, WO2008116831, WO2008116833;
agonists of estrogen receptor gamma (ERRγ agonists), as described, for example, in WO2007131005, WO2008052709;
agonists of estrogen receptor alpha (ERRα/ERR1 agonists), as described, for example, in WO2008109727;
sigma-1 receptor antagonists, as described, for example, in WO2007098953, WO2007098961, WO2008015266, WO2008055932, WO2008055933;
muscarin 3 receptor (M3R) antagonists, as described, for example, in WO2007110782, WO2008041184;
bombesin receptor agonists (BRS-3 agonists), as described, for example, in WO2008051404, WO2008051405, WO2008051406, WO2008073311;
galanin receptor antagonists;
growth hormone (e.g. human growth hormone or AOD-9604);
growth hormone releasing compounds (tert-butyl 6-benzyloxy-1-(2-diisopropylaminoethylcarbamoyl)-3,4-dihydro-1H-isoquinoline-2-carboxylate (WO 01/85695));
growth hormone secretagogue receptor antagonists (ghrelin antagonists), for example A-778193, or those as described in WO2005030734, WO2007127457, WO2008008286;
growth hormone secretagogue receptor modulators (ghrelin modulators), for example JMV-2959, JMV-3002, JMV-2810, JMV-2951, or those as described in WO2006012577 (e.g. YIL-781 or YIL-870), WO2007079239, WO2008092681;
TRH agonists (see, for example, EP 0 462 884);
decoupling protein 2 or 3 modulators;
chemical decouplers (e.g. WO2008059023, WO2008059024, WO2008059025, WO2008059026);
leptin agonists (see, for example, Lee, Daniel W.; Leinung, Matthew C.; Rozhayskaya-Arena, Marina; Grasso, Patricia. Leptin agonists as a potential approach to the treatment of obesity. Drugs of the Future (2001), 26(9), 873-881);
DA agonists (bromocriptin, doprexin);
lipase/amylase inhibitors (e.g. WO 00/40569, WO2008107184);
inhibitors of diacylglycerol O-acyltransferases (DGATs), for example BAY-74-4113, or as described, for example, in US2004/0224997, WO2004094618, WO200058491, WO2005044250, WO2005072740, JP2005206492, WO2005013907, WO2006004200, WO2006019020, WO2006064189, WO2006082952, WO2006120125, WO2006113919, WO2006134317, WO2007016538, WO2007060140, JP2007131584, WO2007071966, WO2007126957, WO2007137103, WO2007137107, WO2007138304, WO2007138311, WO2007141502, WO2007141517, WO2007141538, WO2007141545, WO2007144571, WO2008011130, WO2008011131, WO2008039007, WO2008048991, WO2008067257, WO2008099221;
inhibitors of monoacylglycerol acyltransferase (2-acylglycerol O-acyltransferase; MGAT), as described, for example, in WO2008038768;
inhibitors of fatty acid synthase (FAS), for example C75, or those as described in WO2004005277, WO2008006113;
inhibitors of stearoyl-CoA delta9 desaturase (SCD1), as described, for example, in WO2007009236, WO2007044085, WO2007046867, WO2007046868, WO20070501124, WO2007056846, WO2007071023, WO2007130075, WO2007134457, WO2007136746, WO2007143597, WO2007143823, WO2007143824, WO2008003753, WO2008017161, WO2008024390, WO2008029266, WO2008036715, WO2008043087, WO2008044767, WO2008046226, WO2008056687, WO2008062276, WO2008064474, WO2008074824, WO2008074832, WO2008074833, WO2008074834, WO2008074835, WO2008089580, WO2008096746, WO2008104524, WO2008116898, US2008249100, WO2008120744, WO2008120759, WO2008123469, WO2008127349;
inhibitors of fatty acid desaturase 1 (deltas desaturase), as described, for example, in WO2008089310;
hypoglycemic/hypertriglyceridemic indoline compounds, as described in WO2008039087;
inhibitors of “adipocyte fatty acid-binding protein aP2”, for example BMS-309403; activators of adiponectin secretion, as described, for example, in WO2006082978, WO2008105533;
promoters of adiponectin secretion, as described, for example, in WO2007125946, WO2008038712;
modified adiponectins, as described, for example, in WO2008121009;
oxyntomodulin or analogs thereof;
oleoyl-estrone
or agonists or partial agonists of the thyroid hormone receptor (thyroid hormone receptor agonists), for example: KB-2115 (eprotirome), QRX-431 (sobetirome) or DITPA, or those as described in WO20058279, WO200172692, WO200194293, WO2003084915, WO2004018421, WO2005092316, WO2007003419, WO2007009913, WO2007039125, WO2007110225, WO2007110226, WO2007128492, WO2007132475, WO2007134864, WO2008001959, WO2008106213
or agonists of the thyroid hormone receptor beta (TR-beta), for example MB-07811 or MB-07344, or those as described in WO2008062469.

In one embodiment of the invention, the compound of the formula I is administered in combination with a combination of eprotirome with ezetimibe.

In one embodiment of the invention, the compound of the formula I is administered in combination with an inhibitor of site-1 protease (S1P), for example PF-429242.

In a further embodiment of the invention, the compound of the formula I is administered in combination with a modulator of the “trace amine associated receptor 1” (TAAR1), as described, for example, in US2008146523, WO2008092785.

In one embodiment of the invention, the compound of the formula I is administered in combination with an inhibitor of growth factor receptor bound protein 2 (GRB2), as described, for example, in WO2008067270.

In a further embodiment of the invention, the compound of the formula I is administered in combination with an RNAi (siRNA) therapeutic agent directed against PCSK9 (proprotein convertase subtilisin/kexin type 9).

In one embodiment, the compound of the formula I is administered in combination with Omacor® or Lovaza™ (omega-3 fatty acid ester; highly concentrated ethyl ester of eicosapentaenoic acid and of docosahexaenoic acid).

In one embodiment, the compound of the formula I is administered in combination with lycopene.

In one embodiment of the invention, the compound of the formula I is administered in combination with an antioxidant, for example OPC-14117, AGI-1067 (succinobucol), probucol, tocopherol, ascorbic acid, β-carotene or selenium.

In one embodiment of the invention, the compound of the formula I is administered in combination with a vitamin, for example Vitamin B6 or Vitamin B12.

In one embodiment, the compound of the formula I is administered in combination with more than one of the aforementioned compounds, for example in combination with a sulfonylurea and metformin, a sulfonylurea and acarbose, repaglinide and metformin (PrandiMet™), insulin and a sulfonylurea, insulin and metformin, insulin and troglitazone, insulin and lovastatin, etc.

In another embodiment, the compound of the formula I is administered in combination with an inhibitor of carboanhydrase type 2 (carbonic anhydrase type 2), for example those as described in WO2007065948.

In another embodiment, the compound of the formula I is administered in combination with topiramat or a derivative thereof, as described in WO2008027557.

In a further embodiment, the compound of the formula I is administered in combination with a solid combination of topiramat with phentermin (Qnexa™).

In a further embodiment, the compound of the formula I is administered in combination with an antisense compound, e.g. ISIS-377131, which inhibits the production of the glucocorticoid receptor.

In another embodiment, the compound of the formula I is administered in combination with an aldosterone synthase inhibitor and an antagonist of the glucocorticoid receptor, a cortisol synthesis inhibitor and/or an antagonist of the corticotropin releasing factor, as described, for example, in EP1886695, WO2008119744.

In one embodiment, the compound of the formula I is administered in combination with an agonist of the RUP3 receptor, as described, for example, in WO2007035355, WO2008005576.

In another embodiment, the compound of the formula I is administered in combination with an activator of the gene which codes for ataxia telangiectasia mutated (ATM) protein kinase, for example chloroquine.

In one embodiment, the compound of the formula I is administered in combination with a tau protein kinase 1 inhibitor (TPK1 inhibitor), as described, for example, in WO2007119463.

In one embodiment, the compound of the formula I is administered in combination with a “c-Jun N-terminal kinase” inhibitor (JNK inhibitor), as described, for example, in WO2007125405, WO2008028860, WO2008118626.

In one embodiment, the compound of the formula I is administered in combination with an endothelin A receptor antagonist, for example avosentan (SPP-301).

In one embodiment, the compound of the formula I is administered in combination with modulators of the glucocorticoid receptor (GR), for example KB-3305 or those compounds as described, for example, in WO2005090336, WO2006071609, WO2006135826, WO2007105766, WO2008120661.

In one embodiment, the further active ingredient is varenicline tartrate, a partial agonist of the alpha 4-beta 2 nicotinic acetylcholine receptor.

In one embodiment, the further active ingredient is trodusquemine.

In one embodiment, the further active ingredient is a modulator of the enzyme SIRT1 and/or SIRT3 (an NAD⁺-dependent protein deacetylase); this active ingredient may, for example, be resveratrol in suitable formulations, or those compounds as specified in WO2007019416 (e.g. SRT-1720), WO2008073451.

In one embodiment of the invention, the further active ingredient is DM-71 (N-acetyl-L-cysteine with bethanechol).

In one embodiment, the compound of the formula I is administered in combination with antihypercholesterolemic compounds, as described, for example, in WO2007107587, WO2007111994, WO2008106600, WO2008113796.

In a further embodiment, the compound of the formula I is administered in combination with inhibitors of SREBP (sterol regulatory element-binding protein), as described, for example, in WO2008097835.

In another embodiment, the compound of the formula I is administered in combination with a cyclic peptide agonist of the VPAC2 receptor, as described, for example, in WO2007101146, WO2007133828.

In a further embodiment, the compound of the formula I is administered in combination with an agonist of the endothelin receptor, as described, for example, in WO2007112069.

In a further embodiment, the compound of the formula I is administered in combination with AKP-020 (bis(ethylmaltolato)oxovanadium(IV)).

In another embodiment, the compound of the formula I is administered in combination with tissue-selective androgen receptor modulators (SARM), as described, for example, in WO2007099200, WO2007137874.

In a further embodiment, the compound of the formula I is administered in combination with an AGE (advanced glycation endproduct) inhibitor, as described, for example, in JP2008024673.

In one embodiment of the invention, the further active ingredient is leptin; see, for example, “Perspectives in the therapeutic use of leptin”, Salvador, Javier; Gomez-Ambrosi, Javier; Fruhbeck, Gema, Expert Opinion on Pharmacotherapy (2001), 2(10), 1615-1622.

In another embodiment of the invention, the further active ingredient is metreleptin (recombinant methionyl-leptin) combined with pramlintide.

In a further embodiment of the invention, the further active ingredient is the tetrapeptide ISF-402.

In one embodiment, the further active ingredient is dexamphetamine or amphetamine.

In one embodiment, the further active ingredient is fenfluramin or dexfenfluramin.

In another embodiment, the further active ingredient is sibutramine or those derivatives as described in WO2008034142.

In one embodiment, the further active ingredient is mazindol or phentermin.

In a further embodiment, the further active ingredient is geniposidic acid (WO2007100104) or derivatives thereof (JP2008106008).

In one embodiment, the further active ingredient is a nasal calcium channel blocker, for example diltiazem, or those as described in U.S. Pat. No. 7,138,107.

In one embodiment, the further active ingredient is an inhibitor of sodium-calcium ion exchange, for example those as described in WO2008028958, WO2008085711.

In a further embodiment, the further active ingredient is a blocker of calcium channels, for example of CaV3.2 oder CaV2.2, as described in WO2008033431, WO2008033447, WO2008033356, WO2008033460, WO2008033464, WO2008033465, WO2008033468, WO2008073461.

In one embodiment, the further active ingredient is a modulator of a calcium channel, for example those as described in WO2008073934, WO2008073936.

In one embodiment, the further active ingredient is a blocker of the “T-type calcium channel”, as described, for example, in WO2008033431, WO2008110008.

In one embodiment, the further active ingredient is an inhibitor of KCNQ potassium channel 2 or 3, for example those as described in US2008027049, US2008027090.

In one embodiment, the further active ingredient is an inhibitor of the potassium Kv1.3 ion channel, for example those as described in WO2008040057, WO2008040058, WO2008046065.

In another embodiment, the further active ingredient is a modulator of the MCP-1 receptor (monocyte chemoattractant protein-1 (MCP-1)), for example those as described in WO2008014360, WO2008014381.

In one embodiment, the further active ingredient is a modulator of somatostatin receptor 5 (SSTR5), for example those as described in WO2008019967, US2008064697, US2008249101, WO2008000692.

In one embodiment, the further active ingredient is a modulator of somatostatin receptor 2 (SSTR2), for example those as described in WO2008051272.

In one embodiment, the further active ingredient is an erythropoietin-mimetic peptide which acts as an erythropoietin (EPO) receptor agonist. Such molecules are described, for example, in WO2008042800.

In a further embodiment, the further active ingredient is an anorectic/a hypoglycemic compound, for example those as described in WO2008035305, WO2008035306, WO2008035686.

In one embodiment, the further active ingredient is an inductor of lipoic acid synthetase, for example those as described in WO2008036966, WO2008036967.

In one embodiment, the further active ingredient is a stimulator of endothelial nitric oxide synthase (eNOS), for example those as described in WO2008058641, WO2008074413.

In one embodiment, the further active ingredient is a modulator of carbohydrate and/or lipid metabolism, for example those as described in WO2008059023, WO2008059024, WO2008059025, WO2008059026.

In a further embodiment, the further active ingredient is an angiotensin II receptor antagonist, for example those as described in WO2008062905, WO2008067378.

In one embodiment, the further active ingredient is an agonist of the sphingosine-1-phosphate receptor (S1P), for example those as described in WO2008064315, WO2008074820, WO2008074821.

In one embodiment, the further active ingredient is an agent which retards gastric emptying, for example 4-hydroxyisoleucine (WO2008044770).

In one embodiment, the further active ingredient is a muscle-relaxing substance, as described, for example, in WO2008090200.

In a further embodiment, the further active ingredient is an inhibitor of monoamine oxidase B (MAO-B), for example those as described in WO2008092091.

In another embodiment, the further active ingredient is an inhibitor of the binding of cholesterol and/or triglycerides to the SCP-2 protein (sterol carrier protein-2), for example those as described in US2008194658.

In another embodiment, the further active ingredient is lisofylline, which prevents autoimmune damage to insulin-producing cells.

In one embodiment, the compound of the formula I is administered in combination with bulking agents, preferably insoluble bulking agents (see, for example, Carob/Caromax® (Zunft H J; et al., Carob pulp preparation for treatment of hypercholesterolemia, ADVANCES IN THERAPY (2001 September-October), 18(5), 230-6). Caromax is a carob-containing product from Nutrinova, Nutrition Specialties & Food Ingredients GmbH, Industriepark Höchst, 65926 Frankfurt/Main). Combination with Caromax® is possible in one preparation or by separate administration of compounds of the formula I and Caromax®. Caromax® can in this connection also be administered in the form of food products such as, for example, in bakery products or muesli bars.

It will be appreciated that every suitable combination of the compounds of the invention with one or more of the aforementioned compounds and optionally one or more other pharmacologically active substances is considered to be covered within the scope of protection conferred by the present invention.

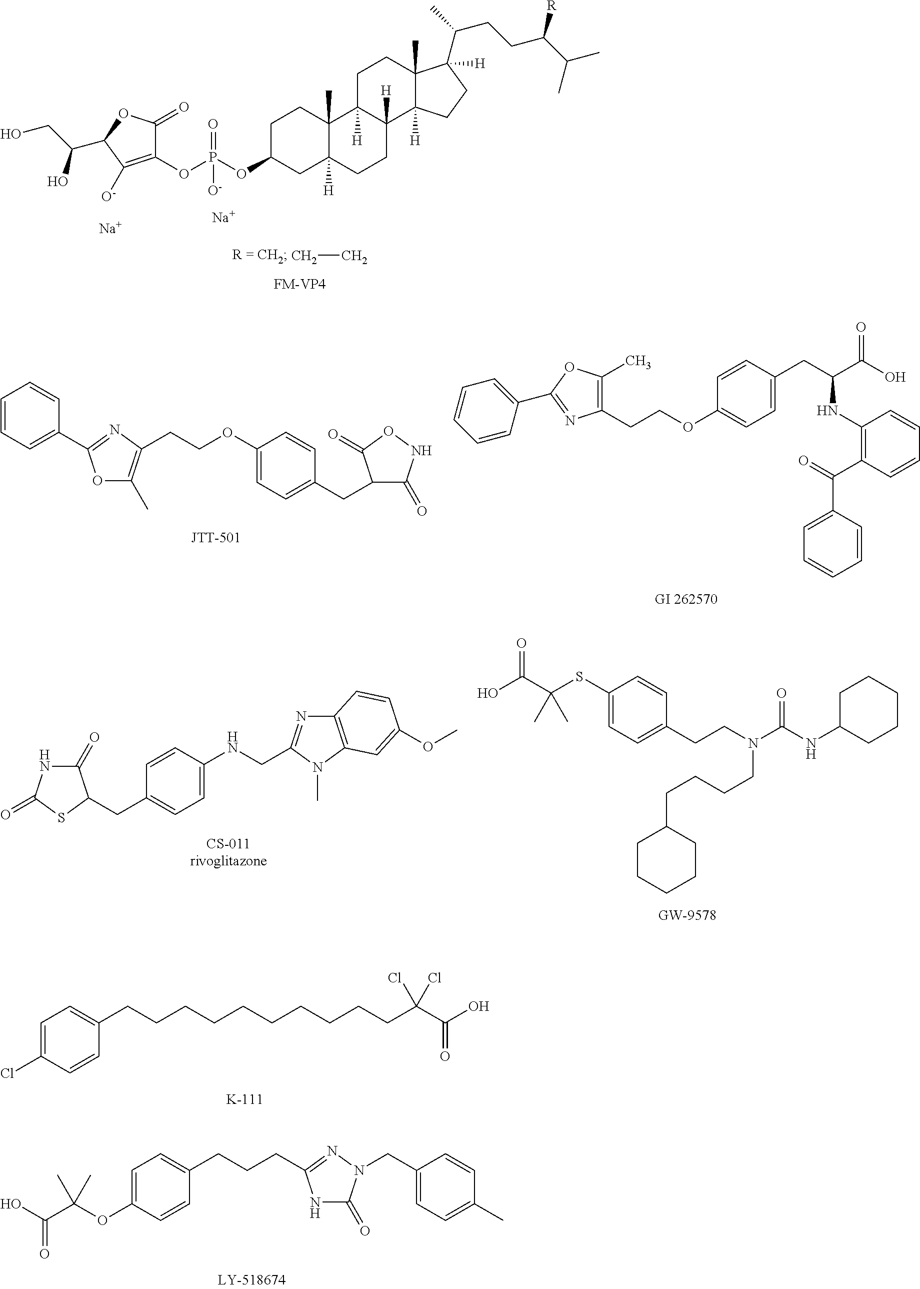

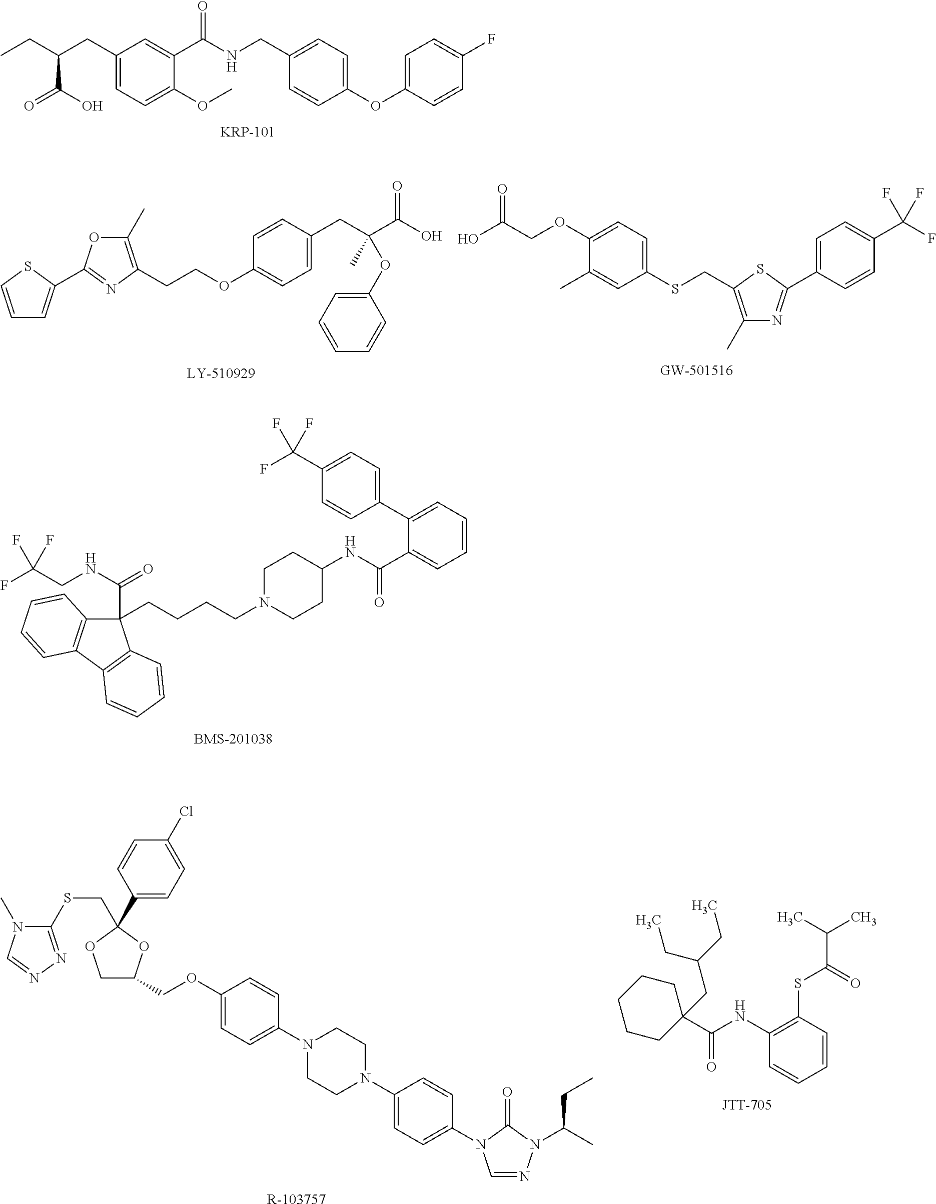

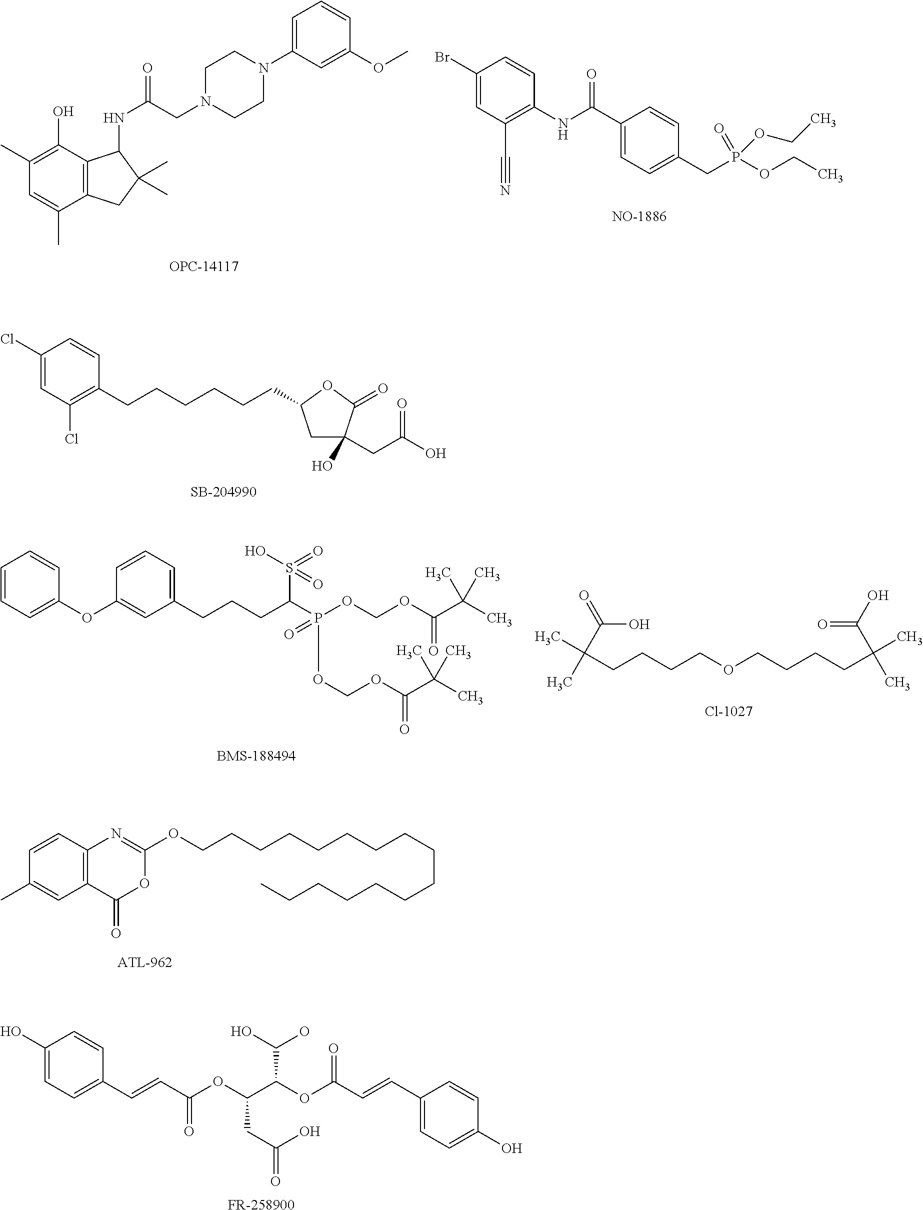

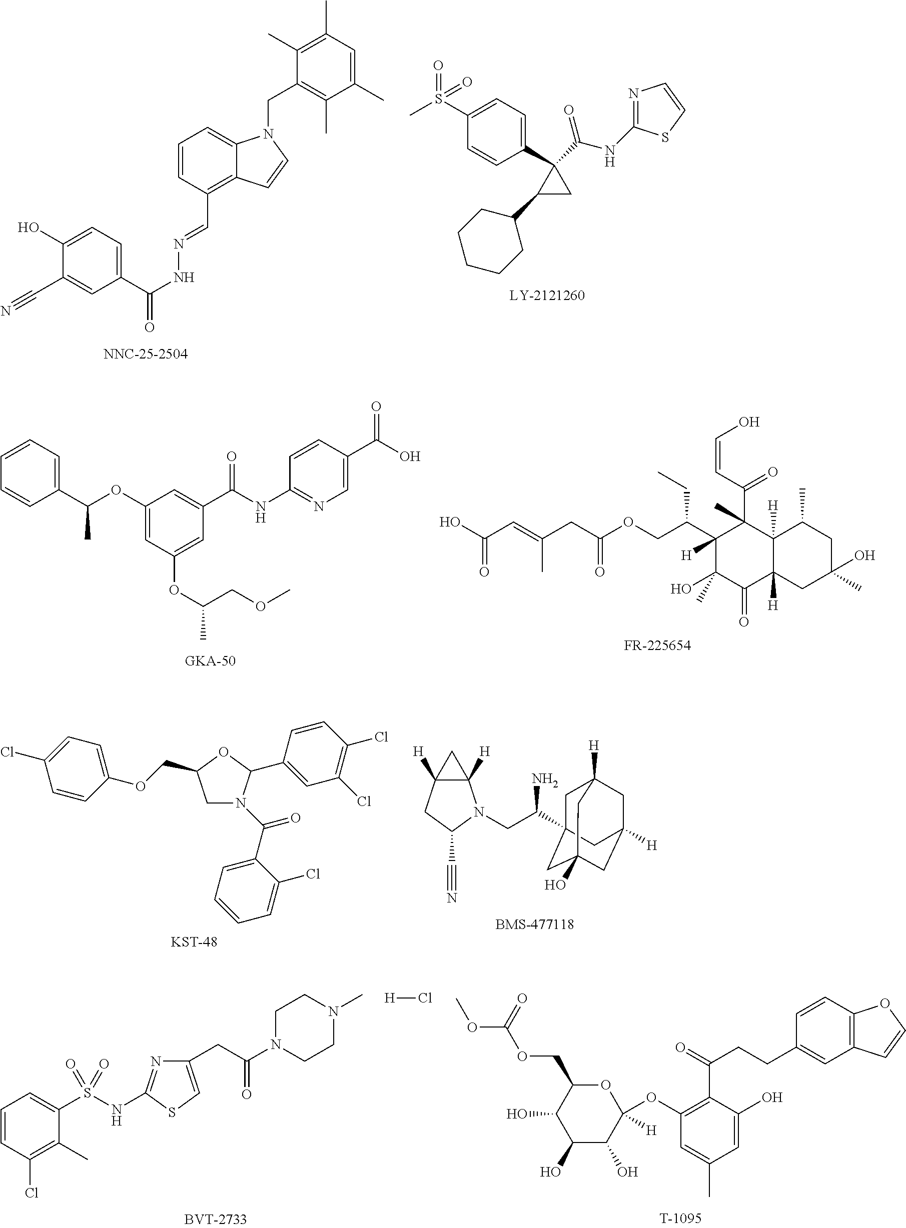

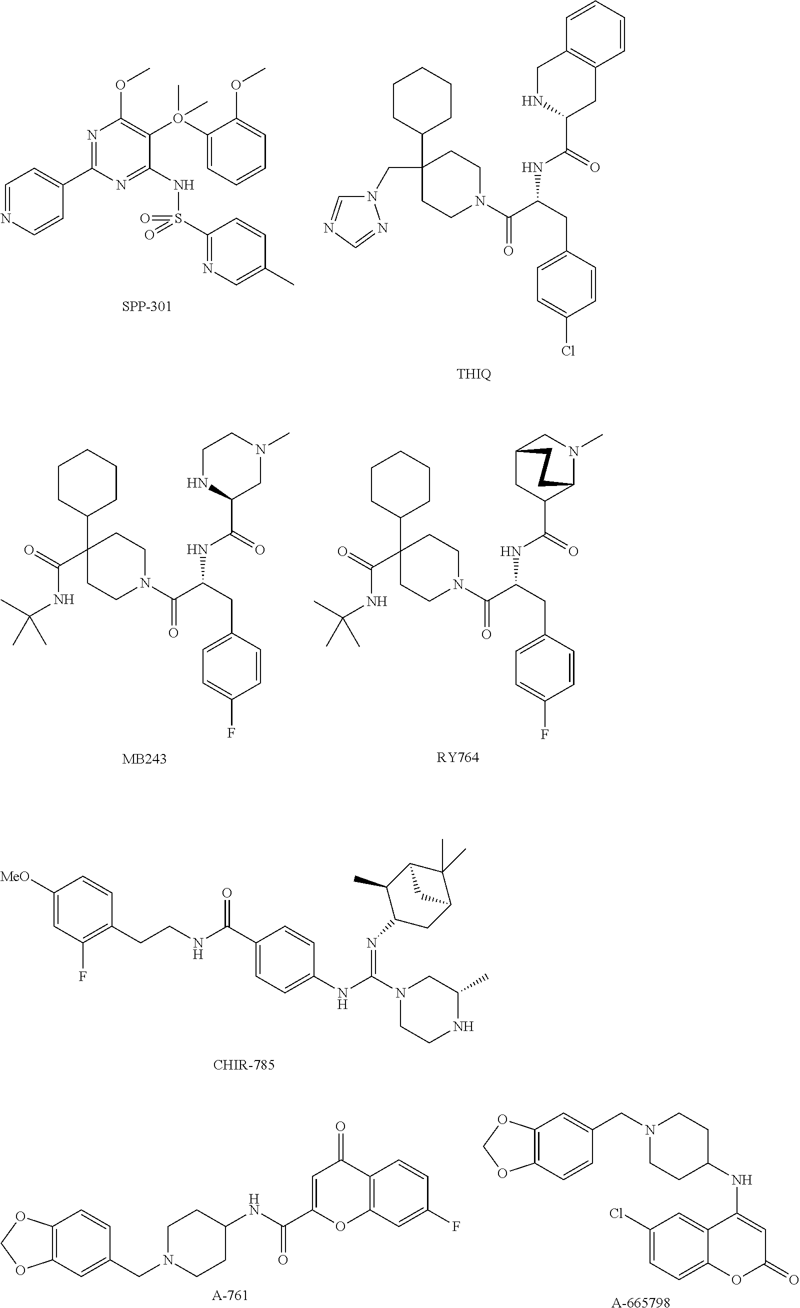

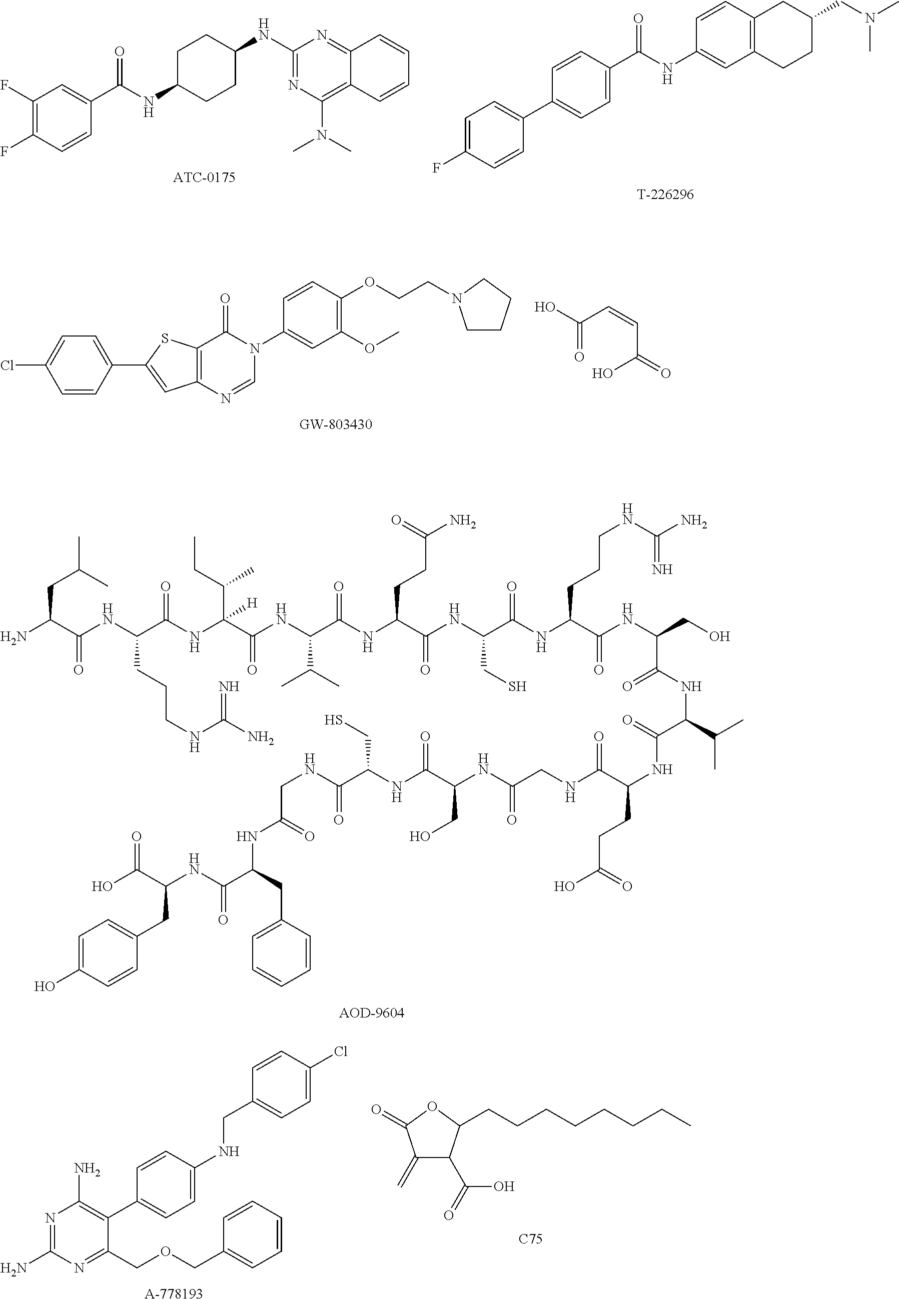

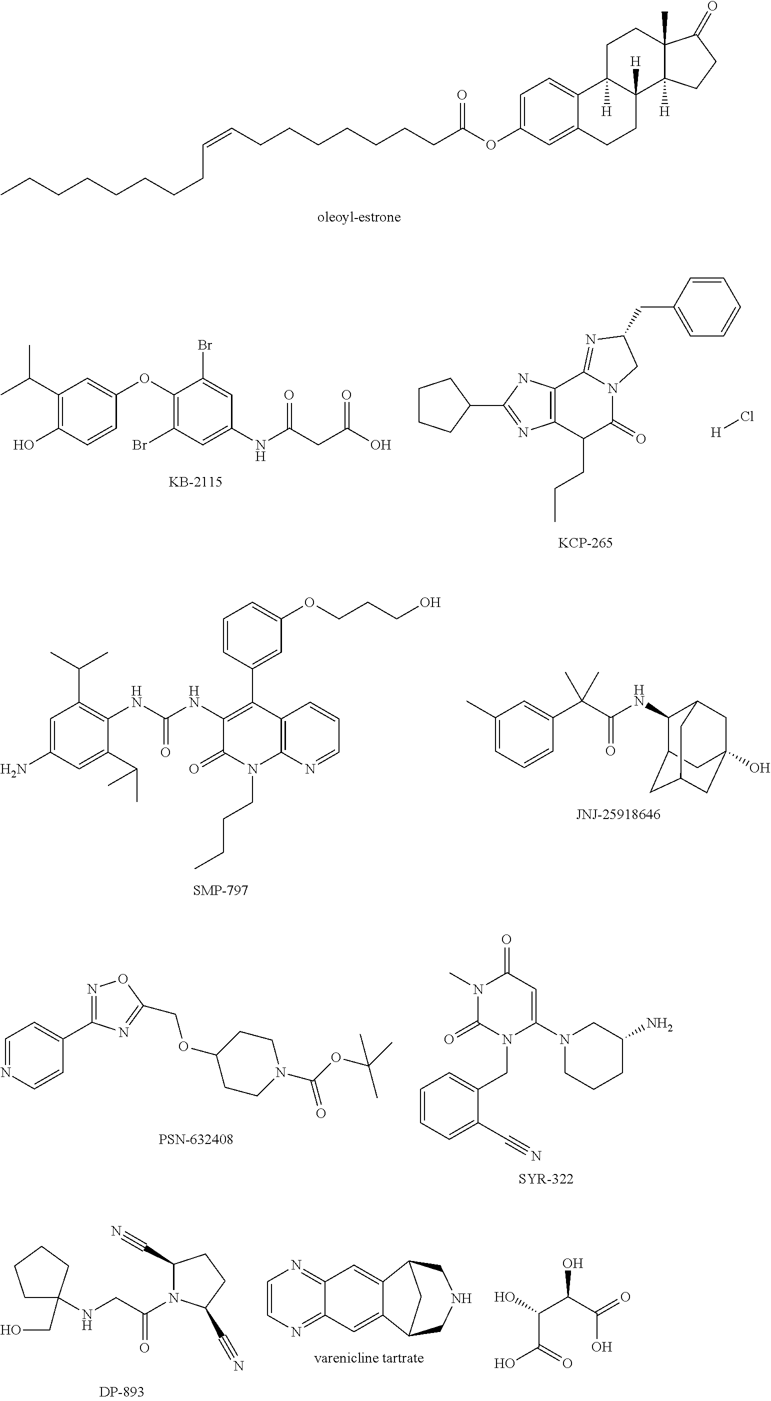

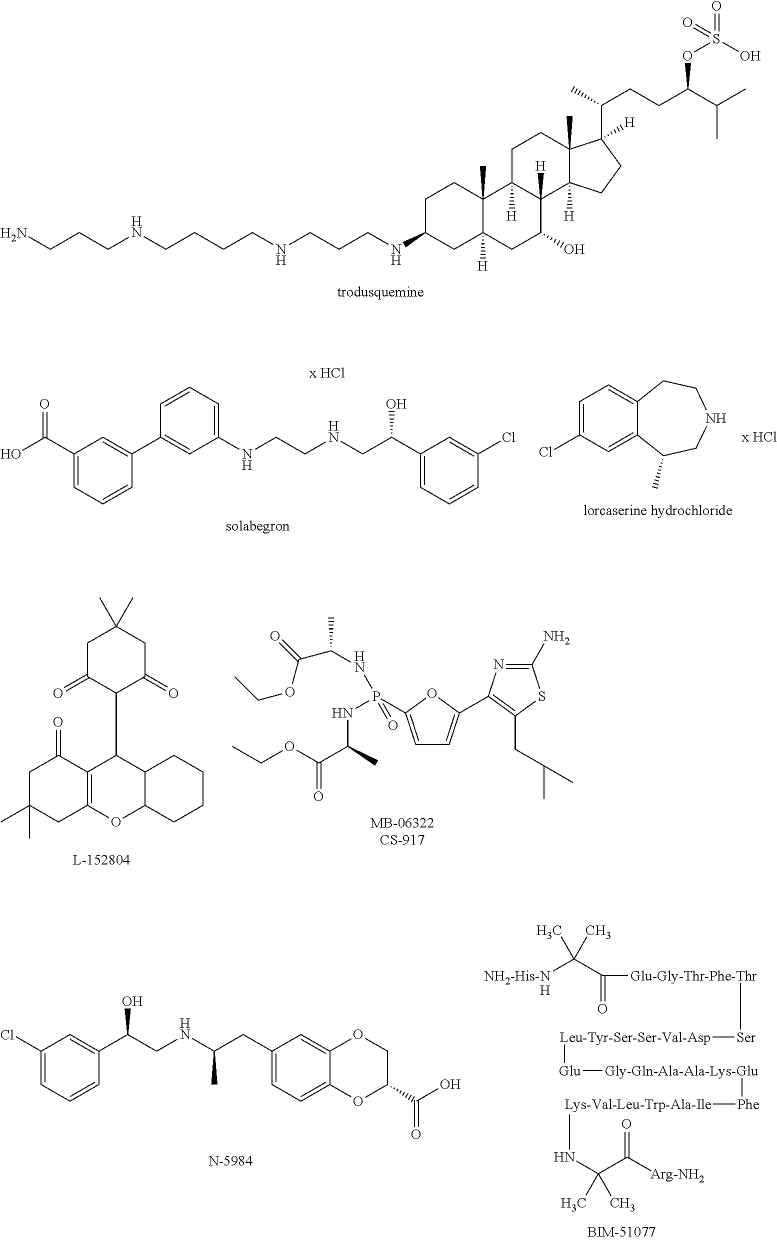

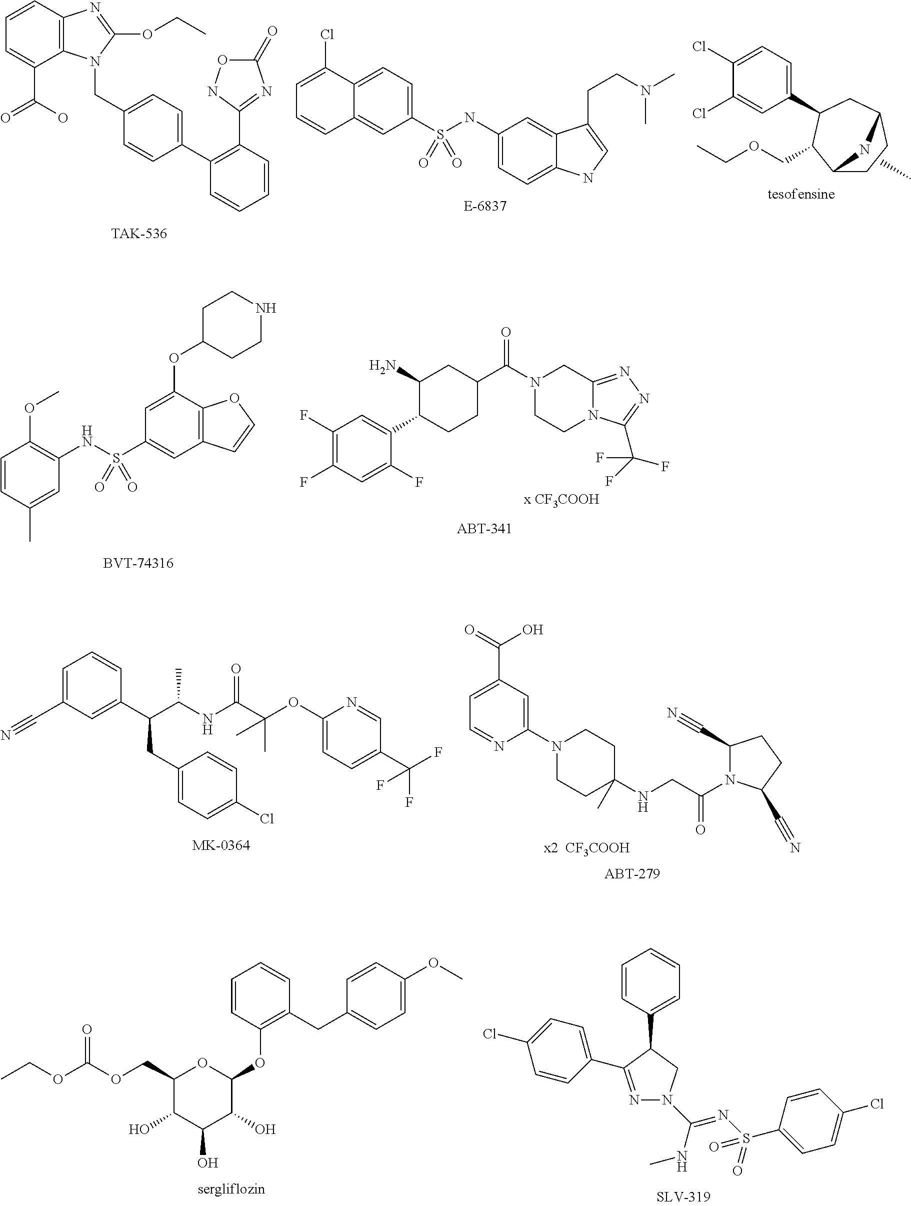

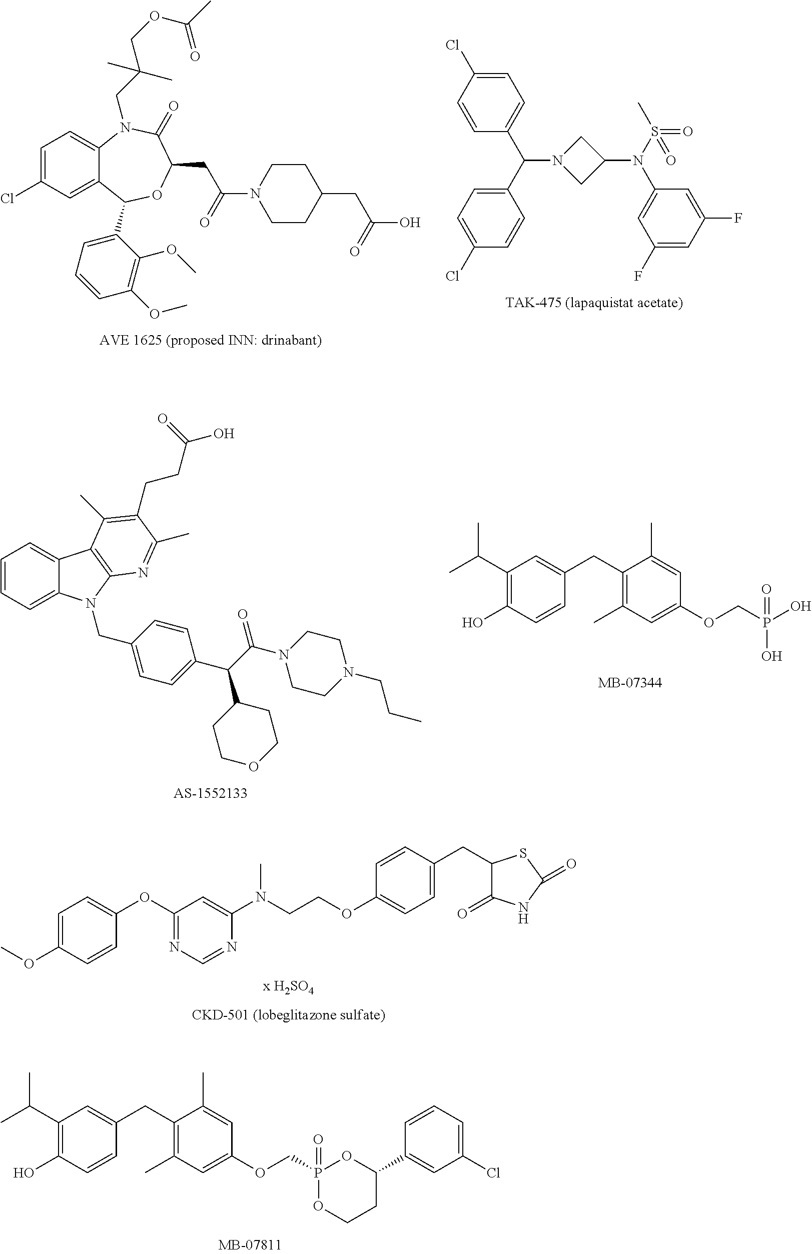

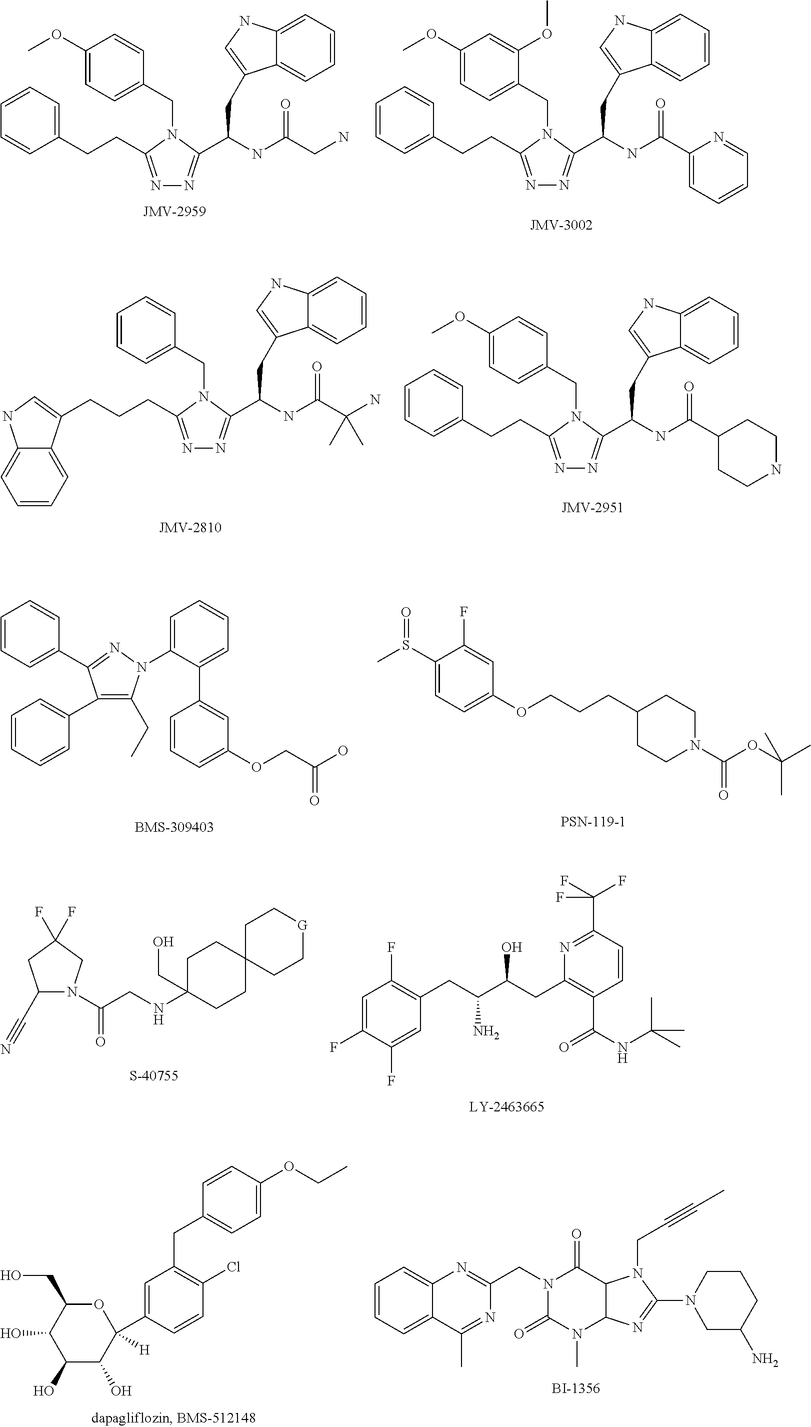

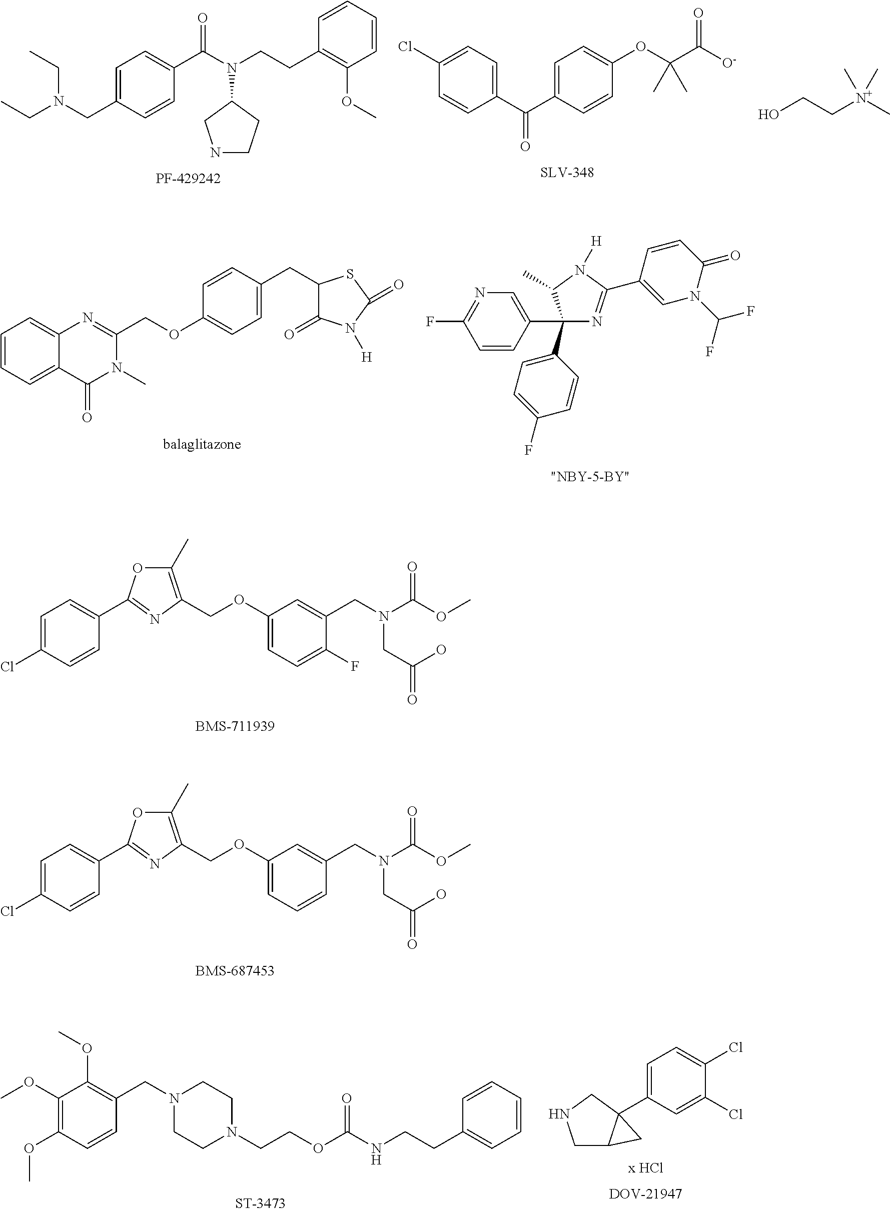

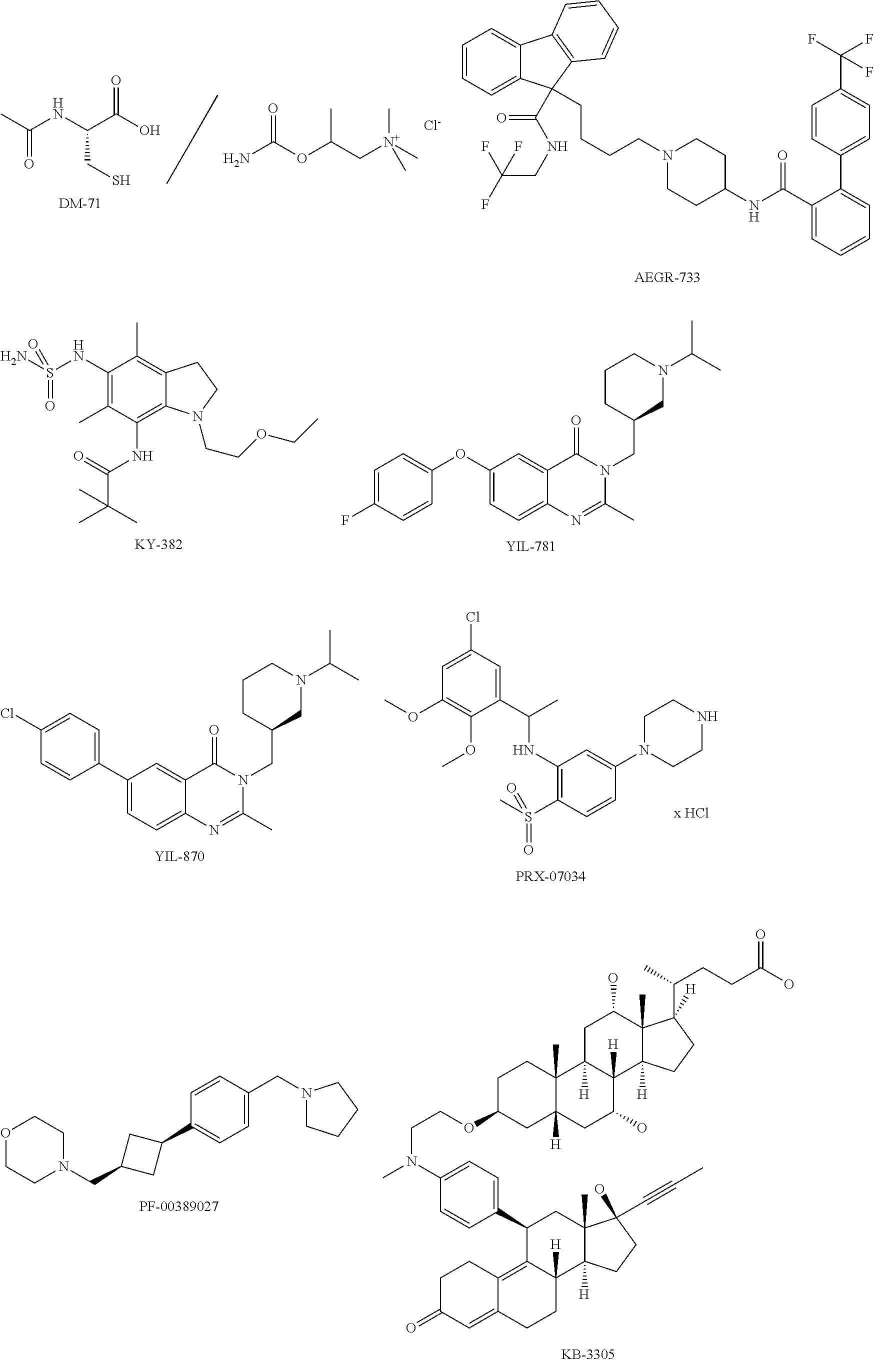

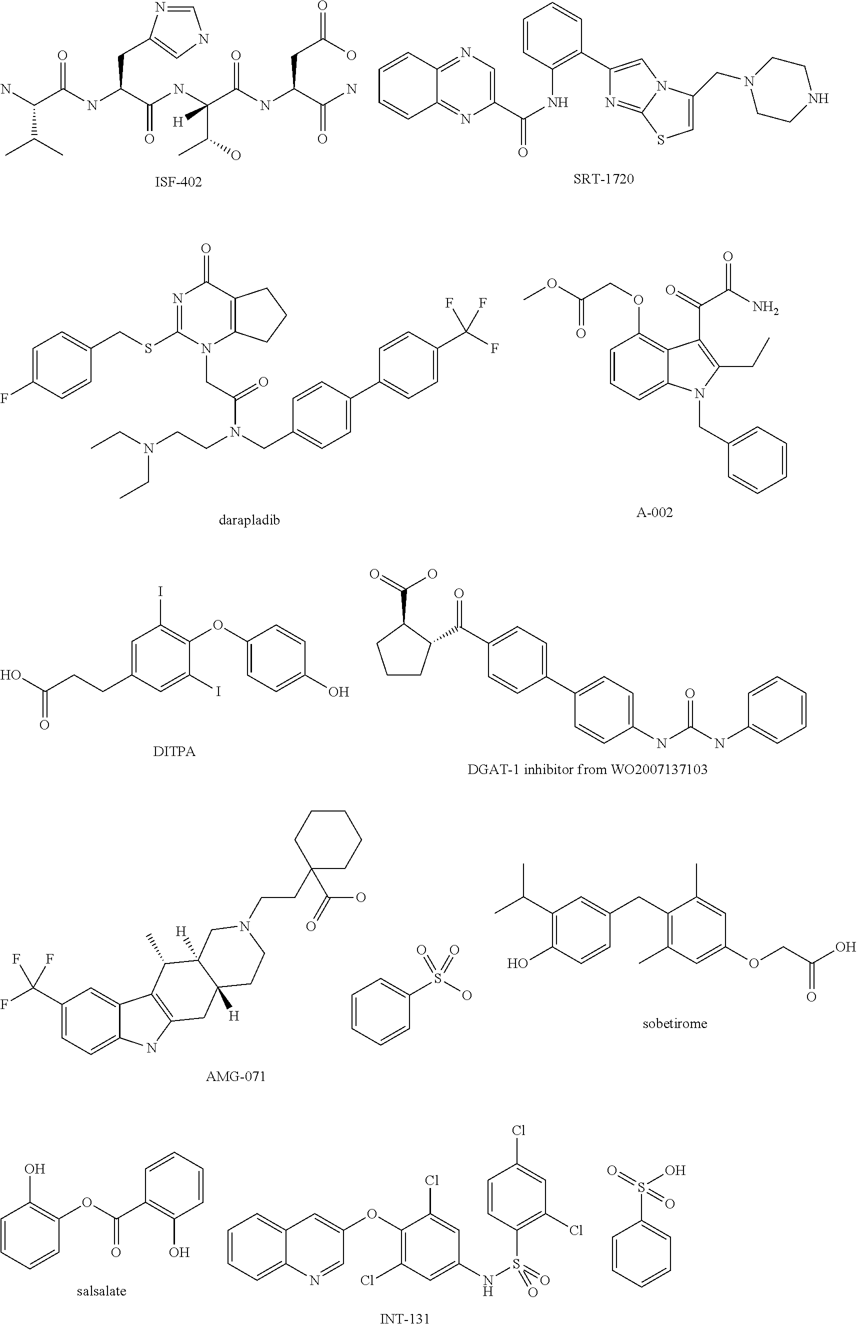

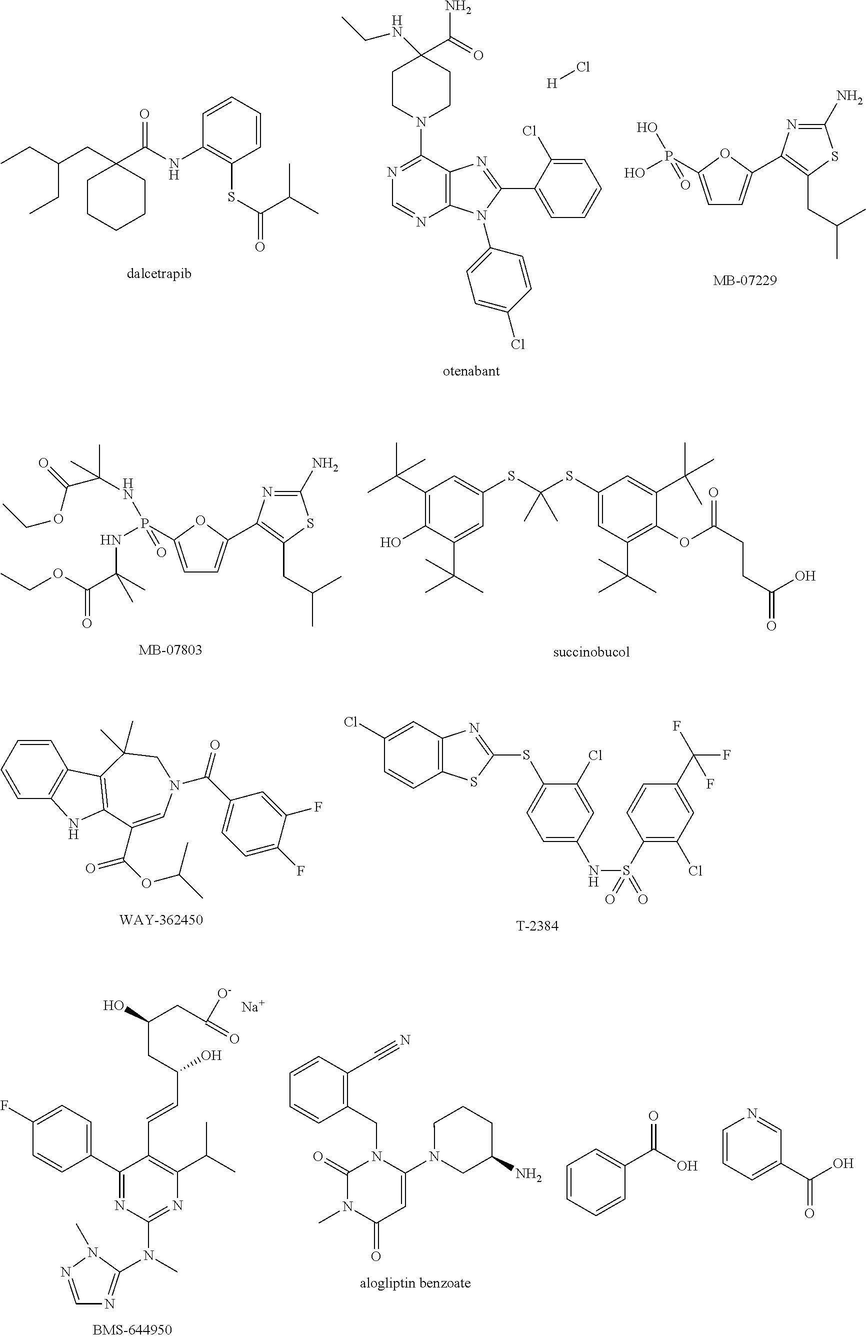

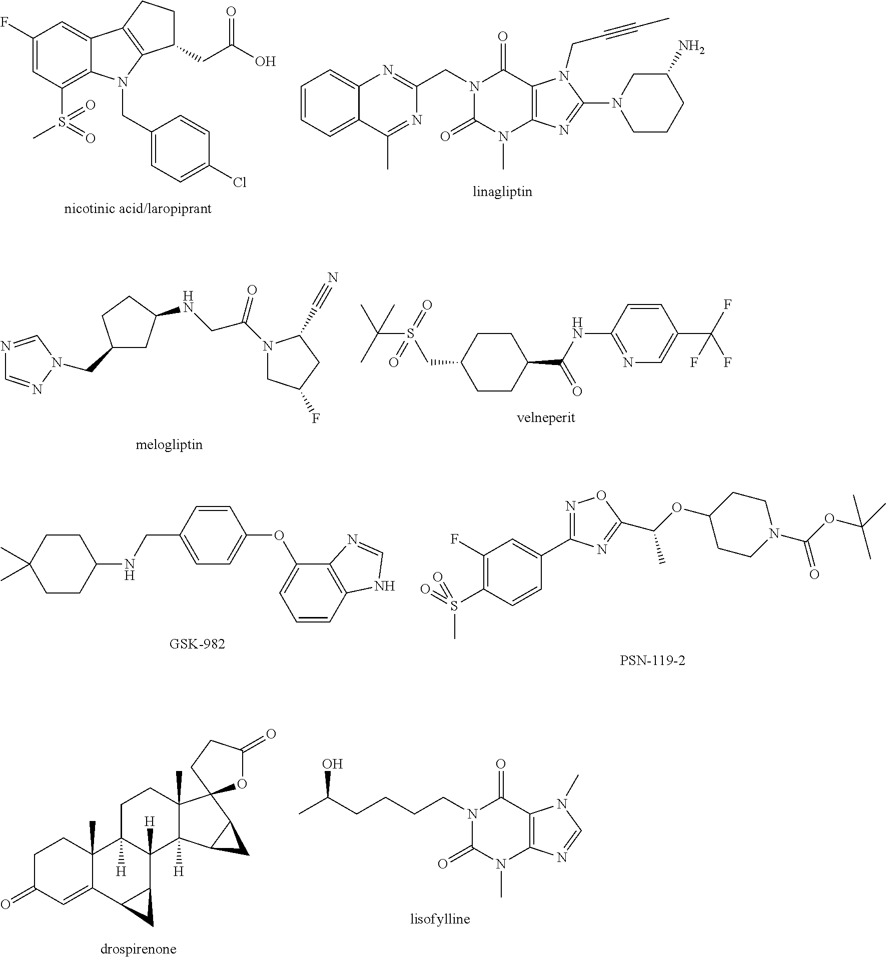

Also suitable are the following active ingredients for combination preparations:

all antiepileptics specified in the Rote Liste 2007, chapter 15;
all antihypertensives specified in the Rote Liste 2007, chapter 17;
all hypotonics specified in the Rote Liste 2007, chapter 19;
all anticoagulants specified in the Rote Liste 2007, chapter 20;
all arteriosclerosis drugs specified in the Rote Liste 2007, chapter 25;
all beta receptors, calcium channel blockers and inhibitors of the renin angiotensin system specified in the Rote Liste 2007, chapter 27;
all diuretics and perfusion-promoting drugs specified in the Rote Liste 2007, chapter 36 and 37;
all withdrawal drugs/drugs for the treatment of addictive disorders specified in the Rote Liste 2007, chapter 39;
all coronary drugs and gastrointestinal drugs specified in the Rote Liste 2007, chapter 55 and 60;
all migraine drugs, neuropathy preparations and Parkinson's drugs specified in the Rote Liste 2007, chapter 61, 66 and 70.

One process according to the invention (“A”) for preparing a compound of the formula I comprises either the reaction of a compound of the formula

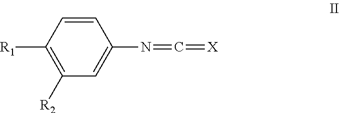

in which R₁, R₂ and X are each as defined above with a compound of the formula

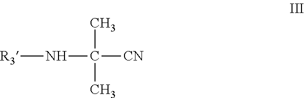

in the presence of a tertiary base, in order to obtain a compound of the formula

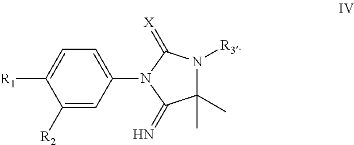

In this compound, R_{3′}, is defined as

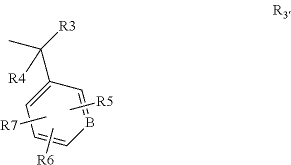

and R₁, R₂ and X are each as defined above.

Compounds of the formula IV can optionally be subjected to one or more of the following reactions in any sequence:
a) hydrolysis reaction of C═NH to give a ketone function or conversion of C═S to C═O
b) conversion reaction of C═O to C═S
c) reaction of the products of the formula IV in which R_{3′} is hydrogen, and, after hydrolysis of C═NH to a ketone, with a compound of the formula R_{3″}-halogen.

A further process (“B”) for preparing the compounds of the formula I

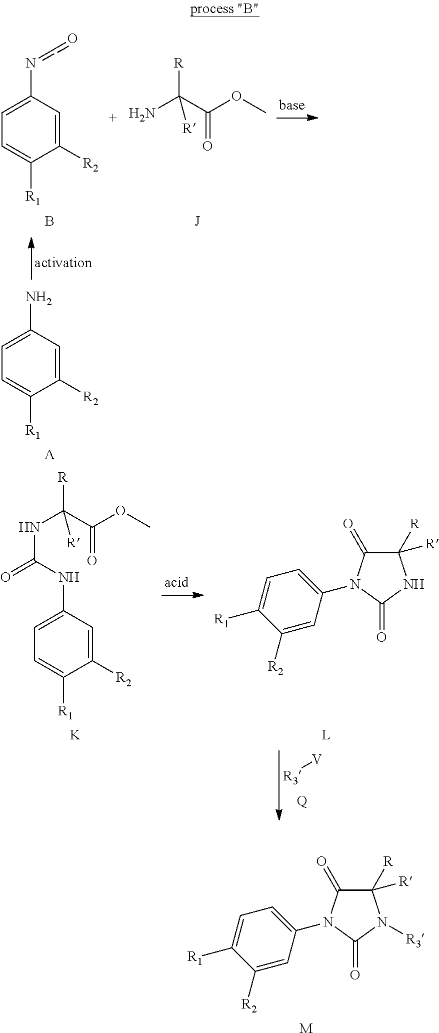

consists in converting a suitably substituted aniline of the formula A in which the R₁, R₂ and R3′ radicals are each defined as described above to an isocyanate of the formula B. This conversion can be performed, for example, with phosgene in toluene or with diphosgene or triphosgene. The isocyanate B is subsequently reacted with the methyl ester or another ester (e.g. tert-butyl) of the amino acid J in which R and R′ are each defined as CH₃, or a salt of an ester of the amino acid J with addition of base (e.g. triethylamine) to give a urea of the formula K. This urea can be ring-closed under basic or acidic conditions, preferably acidic conditions, to give the imidazolidine-2,4-dione of the formula L. The further conversion to a compound of the formula Min which the R₃ radical is defined as described above can be effected, for example, in such a way that L is reacted by alkylation with a suitably substituted compound Q where R₃ is defined as described above and V is halogen, preferably bromine.

In a further process (“C”) for preparing the compounds of the formula I

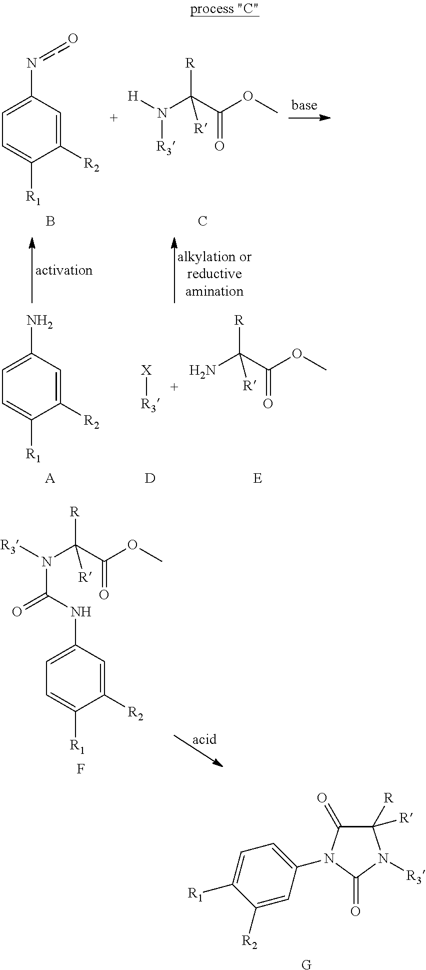

the procedure is that the isocyanate B is reacted with a suitably substituted amino acid ester derivative C, the methyl ester shown in the scheme being a non-limiting example of an ester, and where R₃′, R and R′ are each as defined above, with addition of a base (e.g. triethylamine) to give a urea of the formula F. The amino acid ester derivative C can be prepared from the compound D in which R₃ is defined as described above and X is defined as halogen, preferably bromine, with an amino acid ester of the formula E in which R and R′ are each as defined in formula I, under alkylating conditions. Alternatively, the compound of the formula C can be obtained by reductive amination of the aldehyde D in which R₃ is defined as aryl or heteroaryl and X is defined as (C₀-C₁₁)—CHO with the amino acid derivative E. The urea F can be ring-closed under basic or acidic conditions, preferably acidic conditions, to give the imidazolidine-2,4-dione of the formula G in which R₃ has the definitions described above for formula I.

The optional hydrolysis reaction of C═NH to C═O is preferably conducted with an acid, such as aqueous hydrochloric acid, under reflux. When the hydrolysis of C═NH to C═O is conducted with a molecule which likewise contains C═S, the latter can be converted to a C═O group.

The C═O group is converted to C═S with a Lawesson reagent of the formula

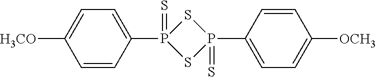

which is a reagent sold commercially, for example by Fluka, and which is described in Bull. Soc. Chim. Belg., Vol. 87 No. 3 (1987), p. 229. When two C═O are converted to C═S, the reaction is conducted in an excess of the Lawesson reagent. The same is also used when the molecule contains both C═S and C═O, and it is desired to convert C═O to C═S. When, in contrast, a portion of the molecule contains two C═O and it is desired to obtain a product with only one C═S, a deficiency of Lawesson reagent is used, in order to obtain a mixture of 3 products, two products with in each case one C═O and C═S and one product with two C═S. These products can be separated by known methods, such as chromatography.

The examples which follow serve to further illustrate the invention, without restricting it to the products and embodiments described in the examples.

General Experimental Methods:

¹H NMR:

The ¹H NMR spectra were measured in deuterated dimethyl sulfoxide on a 500 MHz instrument (DRX 500, from Bruker) at 300 K. Data: δ in ppm, multiplicity (s for singlet, d for doublet, t for triplet, q for quartet, m for multiplet), x H (number of hydrogen atoms)

HPLC-MS:

The HPLC-MS analyses were conducted on a Waters LCT instrument. Column: YMC Jshere 33×2 4 μm; gradient [A]: (acetonitrile+0.05% trifluoroacetic acid):(water+0.05% trifluoroacetic acid) 5:95 (0 minutes) to 95:5 (3 minutes); gradient [B]: (acetonitrile+0.05% trifluoroacetic acid):(water+0.05% trifluoroacetic acid) 5:95 (0 minutes) to 95:5 (2.5 minutes) to 95:5 (3.0 minutes); gradient [C]: (acetonitrile+0.05% trifluoroacetic acid):(water+0.05% trifluoroacetic acid) 5:95 (0 minutes) to 95:5 (3.4 minutes) to 95:5 (4.4 minutes); gradient [D]: (acetonitrile+0.05% trifluoroacetic acid):(water+0.05% trifluoroacetic acid) 5:95 (0 minutes) to 5:95 (0.3 minutes) to 95:5 (3.5 minutes) to 95:5 (4 minutes); gradient [E]: (acetonitrile+0.05% trifluoroacetic acid):(water+0.05% trifluoroacetic acid) 2:98 (1 minute) to 95:5 (5 minutes) to 95:5 (6.25 minutes); detector: Tecan-LCT.

Chromatographic Purification Methods:

[RP1]: flow rate: 30 ml/min; gradient: acetonitrile/water+0.1% trifluoroacetic acid; 30 min. column: XTerra C18 5 μm 30×100 mm; detection: MS (ESI), UV (DAD).
[RP2]: flow rate: 150 ml/min; gradient: acetonitrile/water+0.1% trifluoroacetic acid; 20 min. column: XTerra C18 10 μm 50×250 mm; detection: MS (ESI), UV (DAD).

### EXAMPLE 1

### 4-[3-(3,5-bis(trifluoromethyl)benzyl)-4,4-dimethyl-2,5-dioxoimidazolidin-1-yl]-2-trifluoromethylbenzonitrile

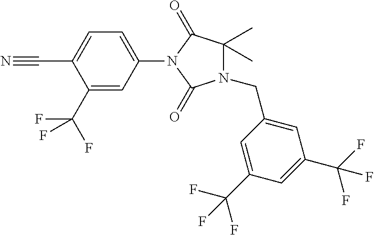

### 1) Preparation of 4-(4,4-dimethyl-5-imino-2-oxo-1-imidazolidinyl)-2-trifluoromethyl-benzonitrile (1.1):

The compound 1.1 can be prepared by process “A”. A solution of 10 g of 4-cyano-3-trifluoromethylaniline (described in European Patent No. 0,002,892) in 30 ml of ethyl acetate was added at from 0 to 5° C. to 33.6 ml of a toluene solution containing 1.93 M/l of phosgene, and, after stirring at from 0 to 5° C. for 30 minutes, the temperature was increased to 25° C. The mixture was distilled while fresh toluene was introduced, which was kept at a constant height, in order to compensate for the distilled volume of toluene, until a temperature of about 110° C. had been attained. The mixture was kept at reflux, until the release of hydrogen chloride decreased (4½ hours). The temperature returned to room temperature, and the white solid was dried over sodium sulfate and rinsed 3 times with toluene. The organic phase was concentrated to dryness under reduced pressure, heated at 60° C. for one hour and then cooled under argon, in order to obtain 11.6 g of 4-isocyanato-2-trifluoromethylbenzonitrile.
A solution of 6.6 g of 4-isocyanato-2-trifluoromethylbenzonitrile in 10 ml of dichloroethane was added at 5° C. to a solution of 2.63 g of 2-amino-2-cyanopropane and 36 ml of dichloroethane and 0.9 ml of triethylamine, and, after stirring at room temperature for 16 hours, the mixture was concentrated to dryness. The 7.7 g of residue were chromatographed on silica gel and eluted with an 85-15 methylene chloride-acetone mixture, in order to obtain 3.54 g of the desired product, which melts at 228° C. An analysis sample was prepared by crystallizing 300 mg from isopropanol, in order to obtain 267 mg of the product, which melts at 228° C.

Analysis: C₁₃H₁₁F₃N₄O; molecular mass = 296.25

| | % C | % H | % F | % N |
|---|---|---|---|---|
| Calculated: | 52.71 | 3.74 | 19.24 | 18.91 |
| Found: | 52.7 | 3.6 | 19.1 | 18.6 |

### 2) Preparation of 4-(4,4-dimethyl-2,5-dioxoimidazolidin-1-yl)-2-trifluoromethylbenzo-nitrile (1.2)

Preparation by process “A”: A solution of 2.76 g of compound 1.1 and 60 ml of 0.5 N hydrochloric acid was heated under reflux for 35 minutes and poured into 100 g of water and ice. The mixture was extracted with ethyl acetate, and the organic phase was washed with water, dried and concentrated to dryness under reduced pressure, in order to obtain 2.70 g of the desired product, which melts at 210° C. An analysis sample was obtained by crystallizing 440 mg of product from isopropanol, in order to obtain 383 mg of product, which melts at 210° C. to 211° C.
Preparation by process “B”: 14.74 g (79.21 mmol) of 4-amino-2-trifluoromethyl-benzonitrile were dissolved in 200 ml of dry acetonitrile. This solution was added dropwise with stirring to a 20% solution, heated to 70° C., of phosgene in toluene and then stirred for 1 h. The cooled reaction solution was concentrated under reduced pressure, and the residue was taken up with toluene and concentrated again under reduced pressure. Finally, the residue was dissolved in 150 ml of dry acetonitrile and the solution was admixed with stirring with 15.5 g (79.21 mmol) of tert-butyl 2-amino-2-methylpropionate hydrochloride. 12.02 g (118.8 mmol) of triethylamine were slowly added dropwise to the reaction mixture and then it was stirred at room temperature for 45 min. Thereafter, the mixture was admixed cautiously with 50 ml of concentrated hydrochloric acid and stirred at 70° C. for 1 h. The cooled reaction mixture was concentrated under reduced pressure and the residue was admixed with ethyl acetate and water. The organic phase was removed, washed with saturated sodium hydrogencarbonate solution and then with water, dried over sodium sulfate, filtered and concentrated under reduced pressure. The residue was purified by chromatography using silica gel with 2:1 heptane/ethyl acetate. This afforded 21.2 g (90% yield) of 4-(4,4-dimethyl-2,5-dioxoimidazolidin-1-yl)-2-trifluoromethylbenzonitrile 1.2 with melting point 208-211° C.

### 3) Preparation of 4-[3-(3,5-bis(trifluoromethyl)benzyl)-4,4-dimethyl-2,5-dioxoimidazolidin-1-yl]-2-trifluoromethylbenzonitrile (1)

100 mg of compound 1.2 and 103 mg of 3,5-bis(trifluoromethyl)benzyl bromide were dissolved in 2.5 ml of dry acetonitrile, admixed with 110 mg of cesium carbonate and stirred at room temperature for 4 h. For workup, the reaction mixture was admixed with ethyl acetate and water. The organic phase was removed, dried over magnesium sulfate and concentrated under reduced pressure. The residue was purified by chromatography (method [RP1]). This afforded 4-[3-(3,5-bis(trifluoromethyl)benzyl)-4,4-dimethyl-2,5-dioxoimidazolidin-1-yl]-2-trifluoromethylbenzonitrile; ¹H NMR: 8.35; d, 1H, 8.23; s, 1H, 8.19; s, 2H, 8.1; d, 1H, 8.03; s, 1H, 4.81; s, 2H, 1.42; s, 6H.

### EXAMPLE 2

### 4-[4,4-dimethyl-2,5-dioxo-3-(4-trifluoromethoxybenzyl)imidazolidin-1-yl]-2-trifluoromethylbenzonitrile

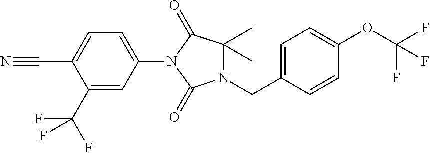 [0334] The compound of example 2 was prepared analogously by reacting 1.2 with 4-(trifluoromethoxy)benzyl bromide. ¹H NMR: 8.35; d, 1H, 8.23; s, 1H, 8.09; d, 1H, 7.58; d, 2H, 7.32; d, 2H, 4.63; s, 2H, 1.41; s, 6H.

### EXAMPLE 3

### 4-[4,4-dimethyl-2,5-dioxo-3-(3-trifluoromethylbenzyl)imidazolidin-1-yl]-2-trifluoromethylbenzonitrile

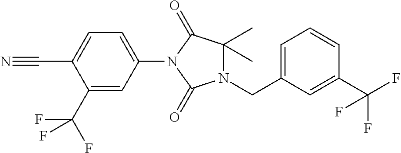 [0336] The compound of example 3 was obtained by reacting 1.2 with 1-bromomethyl-3-trifluoromethylbenzene. ¹H NMR: 8.34; d, 1H, 8.25; s, 1H, 8.1; s, 2H, 7.82; s, 1H, 7.79; d, 1H, 7.61; m, 2H, 4.71; s, 2H, 1.4; s, 6H.

### EXAMPLE 4

### 4-[4,4-dimethyl-2,5-dioxo-3-(4-pentafluorosulfanylbenzyl)imidazolidin-1-yl]-2-trifluoromethylbenzonitrile

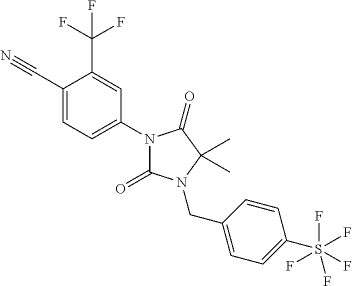

### 1) Preparation of (4-pentafluorosulfanylphenyl)methanol (4.1)

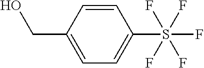 [0339] 10 g of 4-(pentafluorosulfanyl)benzoic acid were dissolved in 100 ml of dry tetrahydrofuran and admixed dropwise, at a temperature of from −5 to 0° C., within 30 min, with 48.4 ml of a 1 M solution of lithium aluminum hydride in tetrahydrofuran. Thereafter, the mixture was allowed to warm slowly to room temperature and was stirred at room temperature for another hour. For workup, the reaction mixture was adjusted cautiously to pH 3 with 2 N hydrochloric acid while cooling. The mixture was filtered, admixed with 300 ml of ethyl acetate and extracted by shaking The organic phase was removed, dried over magnesium sulfate and purified by chromatography (silica gel; 2/1 n-heptane/ethyl acetate). This afforded 7.66 g (81% yield) of (4-pentafluorosulfanylphenyl)methanol as the main product; ¹H NMR: 7.86; d, 2H, 7.53; d, 2H, 5.45; t, 1H, 4.6; d, 2H.
A by-product isolated was 165 mg of (4-mercaptophenyl)methanol; ¹H NMR: 7.25; d, 2H, 7.19; d, 2H, 5.3; s, 1H, 5.11; t, 1H, 4.41; d, 2H.

### 2) Preparation of 1-bromomethyl-4-pentafluorosulfanylbenzene (4.2)

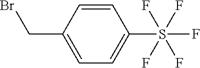 [0342] 6.23 g of triphenylphosphine and 1.93 g of imidazole were initially charged in 60 ml of dichloromethane; 3.79 g of bromine, dissolved in 10 ml of dichloromethane, were added dropwise to this mixture at 5° C. with stirring. The mixture was stirred at 5° C. for 10 min. Subsequently, at 5° C., a solution of 5.3 g of compound 4.1 in 60 ml of dichloromethane was slowly added dropwise with stirring. After the addition had ended, the mixture was stirred at 5° C. for a further hour; after warming to room temperature, the mixture was stirred for a further 3 hours. For workup, the mixture was admixed with 60 ml of 1 N hydrochloric acid; the organic phase was removed, washed with water, dried over magnesium sulfate and concentrated under reduced pressure. The residue was admixed with 150 ml of diethyl ether, stirred, filtered and concentrated again. The residue was then purified by chromatography (silica gel; 3/1 n-heptane/ethyl acetate). This afforded 1-bromomethyl-4-pentafluorosulfanylbenzene (4.2); ¹H NMR: 7.91; d, 2H, 7.69; d, 2H, 4.8; s, 2H.

### 3) Preparation of 4-[4,4-dimethyl-2,5-dioxo-3-(4-pentafluorosulfanylbenzyl)-imidazolidin-1-yl]-2-trifluoromethylbenzonitrile

The compound of example 4 was obtained by reacting 1.2 with 4.2 analogously to the procedure in the preparation of the compound of example 1. ¹H NMR: 8.35; d, 1H, 8.25; s, 1H, 8.09; d, 1H, 7.89; d, 2H, 7.7; d, 2H, 4.7; s, 2H, 1.42; s, 6H.

### EXAMPLE 5

### 4-[3-(6-chloropyridin-3-ylmethyl)-4,4-dimethyl-2,5-dioxoimidazolidin-1-yl]-2-trifluoromethylbenzonitrile

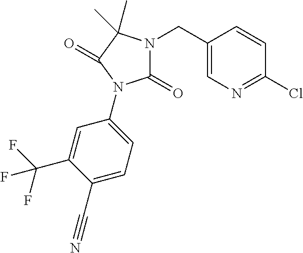 [0345] The compound of example 5 was obtained by reacting 1.2 with 2-chloro-5-chloromethylpyridine similarly to the procedure in the preparation of the compound of example 1. The solvent used was dimethylformamide and the base sodium hydride. ¹H NMR: 8.51; s, 1H, 8.33; d, 1H, 8.23; s, 1H, 8.07; d, 1H, 7.94; d, 1H, 4.67; s, 2H, 1.42; s, 6H.

### EXAMPLE 6

### 4-[4,4-dimethyl-2,5-dioxo-3-(6-trifluoromethylpyridin-3-ylmethyl)-imidazolidin-1-yl]-2-trifluoromethylbenzonitrile

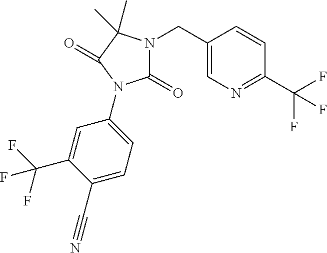 [0347] The compound of example 6 was obtained by reacting 1.2 with 3-(chloromethyl)-6-(trifluoromethyl)pyridine similarly to the procedure in the preparation of the compound of example 1. The solvent used was dimethylformamide and the base sodium hydride. ¹H NMR: 8.87; s, 1H, 8.33; d, 1H, 8.23; s, 1H, 8.09; d, 1H, 7.9; d, 1H, 4.79; s, 2H, 1.46; s, 6H.

### EXAMPLE 7

### 3-(4-fluoro-3-trifluoromethylphenyl)-5,5-dimethyl-1-(6-trifluoromethyl-pyridin-3-ylmethyl)imidazolidine-2,4-dione

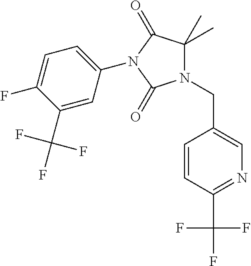 [0349] The compound of example 7 can be prepared by process “C”:

### 1) Preparation of N-methoxy-N-methyl-6-trifluoromethylnicotinamide (7.1)

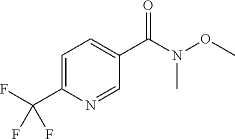 [0351] 1.5 g of 6-trifluoromethylnicotinic acid and 0.84 g of N,O-dimethylhydroxylamine hydrochloride were dissolved in 30 ml of dichloromethane, and the solution was admixed with 4.99 g of 2,4,6-tripropyl-[1,3,5,2,4,6]trioxatriphosphinane 2,4,6-trioxide and 1.59 g of triethylamine and then stirred at room temperature for 6 h. For workup, the reaction mixture was concentrated under reduced pressure, and the residue was taken up in 50 ml of ethyl acetate and extracted by shaking twice with 25 ml each time of sodium hydrogensulfate solution and twice with 25 ml each time of saturated sodium carbonate solution. The organic phase was removed, dried over magnesium sulfate, filtered and concentrated under reduced pressure. This afforded N-methoxy-N-methyl-6-trifluoromethylnicotinamide (7.1). ¹H NMR: 8.94; s, 1H, 8.3; d, 1H, 8.01; d, 1H, 3.58; s, 3H, 3.34; s, 3H.

### 2) Preparation of 6-trifluoromethylpyridine-3-carbaldehyde (7.2)

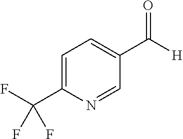 [0353] 1.62 g of compound 7.1 were dissolved in 35 ml of dry tetrahydrofuran and admixed dropwise at −60° C. while stirring with 6.9 ml of a 1-molar solution of lithium aluminum hydride in tetrahydrofuran. After the addition had ended, the mixture was stirred at −60° C. for another hour; then the mixture was allowed to warm to room temperature. For workup, the reaction mixture was admixed dropwise while stirring with 40 ml of cold potassium hydrogensulfate solution. The mixture was extracted by shaking twice with 50 ml each time of ethyl acetate; the organic phase was washed with saturated sodium chloride solution, dried over magnesium sulfate, filtered and concentrated under reduced pressure. This afforded 6-trifluoromethylpyridine-3-carbaldehyde (7.2). ¹H NMR: 10.2; s, 1H, 9.28; s, 1H, 8.55; d, 1H, 8.18; d, 1H.

### 3) Preparation of methyl 2-methyl-2-{[1-(6-trifluoromethylpyridin-3-yl)methylidene]-amino}propionate (7.3)

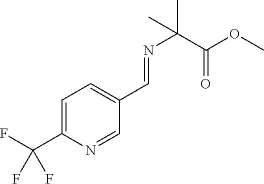 [0355] 1 g of methyl 2-amino-2-methylpropionate hydrochloride was suspended in 20 ml of dichloromethane and admixed dropwise while stirring with 0.66 g of triethylamine. 15 minutes after the addition had ended, the mixture was admixed with 1.57 g of magnesium sulfate and 1.14 g of compound 7.2, and stirred at room temperature for 24 hours. For workup, the reaction mixture was filtered, the filtrate was extracted by shaking with water and saturated sodium chloride solution, and the organic phase was removed, dried over magnesium sulfate, filtered and concentrated under reduced pressure. This afforded methyl 2-methyl-2-{[1-(6-trifluoromethylpyridin-3-yl)-methylidene]amino}propionate (7.3). ¹H NMR: 9.1; s, 1H, 8.56; s, 1H, 8.44; d, 1H, 8.0; d, 1H, 3.69; s, 3H, 1.5; s, 6H.

### 4) Preparation of methyl 2-methyl-2-[(6-trifluoromethylpyridin-3-ylmethyl)amino]-propionate (7.4)

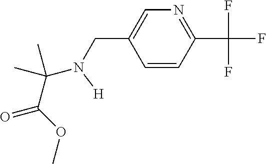 [0357] 1.7 g of compound 7.3 were dissolved in a mixture of 7.5 ml of dry dichloromethane and 7.5 ml of dry methanol, admixed with 66 mg of palladium-on-carbon (10%) and hydrogenated at 1 bar until the absorption of hydrogen had ended. For workup, the catalyst was filtered off, the filtrate was concentrated under reduced pressure and the residue was purified by chromatography (silica gel; 10/1 dichloromethane/methanol). This afforded methyl 2-methyl-2-[(6-trifluoromethylpyridin-3-ylmethyl)-amino]propionate (7.4). Molecular weight 276.10 (C₁₂H₁₅F₃N₂O₂); retention time Rₜ=0.84 min. [C]; MS (ESI): 277.13 (MH⁺).

### 5) Preparation of 3-(4-fluoro-3-trifluoromethylphenyl)-5,5-dimethyl-1-(6-trifluoromethylpyridin-3-ylmethyl)imidazolidine-2,4-dione

0.15 mmol of the amino acid ester 7.4 were dissolved in 1 ml of dry acetonitrile, admixed with 0.165 mmol of 1-fluoro-4-isocyanato-2-trifluoromethylbenzene and stirred overnight at room temperature with exclusion of moisture. After the reaction had ended, the mixture was admixed with 100 μl of concentrated hydrochloric acid and stirred until complete ring closure for 3 h. Thereafter, the solvent was removed under reduced pressure and the residue was purified by chromatography (method [RP1]). This afforded 3-(4-fluoro-3-trifluoromethylphenyl)-5,5-dimethyl-1-(6-trifluoromethylpyridin-3-ylmethyl)imidazolidine-2,4-dione (7). Molecular weight 449.09 (C₁₉H₁₄F₇N₃O₂); retention time Rₜ=2.03 min. [B]; MS (ESI): 450.25 (MH⁺).

### EXAMPLE 8

### 3-(4-chloro-3-trifluoromethylphenyl)-5,5-dimethyl-1-(6-trifluoromethyl-pyridin-3-ylmethyl)imidazolidine-2,4-dione

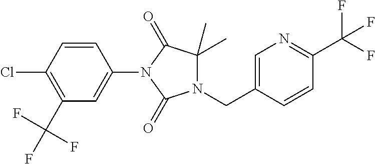 [0360] The compound of example 8 was prepared like the compound of example 7, with the difference that the compound 7.4 was not reacted with 1-fluoro-4-isocyanato-2-trifluoromethylbenzene but with 1-chloro-4-isocyanato-2-trifluoromethylbenzene. Molecular weight 465.06 (C₁₉H₁₄ClF₆N₃O₂); retention time Rₜ=2.13 min. [B]; MS (ESI): 466.24 (MH⁺).

The compounds of examples 21 (3-(3,4-difluorophenyl)-5,5-dimethyl-1-(6-trifluoromethylpyridin-3-ylmethyl)imidazolidine-2,4-dione,

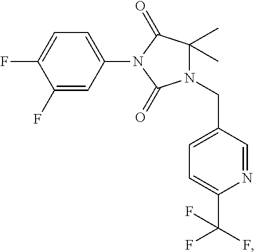

¹H NMR: 8.85; s, 1H, 8.13; d, 1H, 7.9; d, 1H, 7.6; m, 2H, 7.4; m, 1H, 4.76; s, 2H, 1.41; s, 6H) and 22 (3-(3,4-dichlorophenyl)-5,5-dimethyl-1-(6-trifluoromethylpyridin-3-ylmethyl)imidazolidine-2,4-dione,

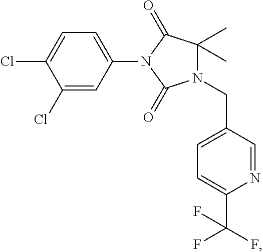

¹H NMR: 8.85; s, 1H, 8.16; d, 1H, 7.9; d, 1H, 7.8; m, 2H, 7.51; d, 1H, 4.76; s, 2H, 1.41; s, 6H) were obtained analogously by reacting 7.4 with, respectively, 1,2-difluoro-4-isocyanatobenzene and 1,2-dichloro-4-isocyanatobenzene.

### EXAMPLE 9

### 1-(3,5-bis(trifluoromethyl)benzyl)-3-(4-fluoro-3-trifluoromethylphenyl)-5,5-dimethylimidazolidine-2,4-dione

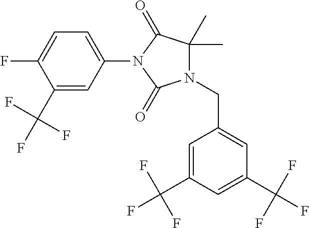 [0365] The compound of example 9 was obtained via an analogous reaction sequence: reaction of 3,5-bis(trifluoromethyl)benzaldehyde with methyl 2-amino-2-methyl-propionate hydrochloride afforded methyl 2-{[1-(3,5-bis(trifluoromethyl)phenyl)-methylidene]amino}-2-methylpropionate (9.3; ¹H NMR: 8.6; s, 1H, 8.45; s, 2H, 8.25; s, 1H, 3.69; s, 3H, 1.5; s, 6H). The hydrogenation thereof afforded the amino acid derivative methyl 2-(3,5-bis(trifluoromethyl)benzylamino)-2-methylpropionate (9.4; molecular weight 343.10 (C₁₄H₁₅F₆NO₂); retention time Rₜ=1.36 min. [C]; MS (ESI): 344.19 (MH⁺)). The further reaction of compound 9.4 with 1-fluoro-4-isocyanato-2-trifluoromethylbenzene gave 1-(3,5-bis(trifluoromethyl)benzyl)-3-(4-fluoro-3-trifluoromethylphenyl)-5,5-dimethylimidazolidine-2,4-dione (9; ¹H NMR: 8.18; s, 2H, 8.03; s, 1H, 7.98; m, 1H, 7.89; m, 1H, 7.7; m, 1H, 4.8; s, 2H, 1.42; s, 6H).

### EXAMPLE 10

### 1-(3,5-bis(trifluoromethyl)benzyl)-3-(4-chloro-3-trifluoromethylphenyl)-5,5-dimethylimidazolidine-2,4-dione

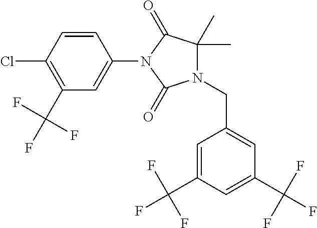 [0367] In an analogous manner, except by reacting with 1-chloro-4-isocyanato-2-trifluoromethylbenzene instead of with 1-fluoro-4-isocyanato-2-trifluoromethyl-benzene, 10 was obtained from 9.4. ¹H NMR: 8.17; s, 2H, 8.08; s, 1H, 8.02; m, 1H, 7.88; m, 2H, 4.8; s, 2H, 1.42; s, 6H).

### EXAMPLE 11

### 4-[4,4-dimethyl-2,5-dioxo-3-(3-pentafluorosulfanylbenzyl)imidazolidin-1-yl]-2-trifluoromethylbenzonitrile

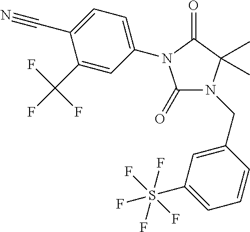 [0369] In the preparation of the compound of example 11, the procedure was as described above for compound 4:
(3-Pentafluorosulfanylphenyl)methanol (11.1; ¹H NMR: 7.83; s, 1H, 7.78; d, 1H, 7.59; m, 2H, 5.5; t, 1H, 4.6; d, 2H) was obtained by lithium aluminum hydride reduction of 3-(pentafluorosulfanyl)benzoic acid. 1-Bromomethyl-3-pentafluorosulfanylbenzene (11.2; ¹H NMR: 8.02; s, 1H, 7.87; d, 1H, 7.78; d, 1H, 7.63; m, 1H, 4.83; s, 2H) was obtained from 11.1 by reaction with phosphorus tribromide in dichloromethane. The alkylating reaction of 1.2 with 11.2 afforded 4-[4,4-dimethyl-2,5-dioxo-3-(3-pentafluorosulfanylbenzyl)imidazolidin-1-yl]-2-trifluoromethylbenzonitrile (11; molecular weight 513.07 (C₂₀H₁₅F₈N₃O₂S); retention time Rₜ=2.19 min. [B]; MS (ESI): 514.16 (MH⁺)).

### EXAMPLE 12

### 3-(4-fluoro-3-trifluoromethylphenyl)-5,5-dimethyl-1-(3-pentafluorosulfanyl-benzyl)imidazolidine-2,4-dione

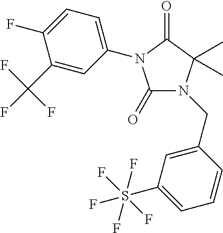

### 1) Preparation of 3-(4-fluoro-3-trifluoromethylphenyl)-5,5-dimethylimidazolidine-2,4-dione (12.1)

Compound 12.1 can be prepared by process “B”. For this purpose, 1.5 g (9.76 mmol) of methyl 2-amino-2-methylpropionate hydrochloride were suspended in 20 ml of dry tetrahydrofuran and admixed with 1.38 ml (9.76 mmol) of triethylamine and 2 g (9.76 mmol) of 1-fluoro-4-isocyanato-2-trifluoromethylbenzene. The mixture was stirred at 70° C. for 1 h; then it was allowed to cool somewhat, 10 ml of concentrated hydrochloric acid were added and the mixture was stirred at 70° C. for 2 h. The cooled reaction mixture was admixed with ethyl acetate and water; the organic phase was removed, dried over sodium sulfate, filtered and concentrated under reduced pressure. The residue was purified by chromatography (method [RP2]) and, after dissolution in ethyl acetate, drying of the solution, concentration under reduced pressure and redissolution in dichloromethane, crystallized with n-heptane. This afforded 2.8 g of 3-(4-fluoro-3-trifluoromethylphenyl)-5,5-dimethylimidazolidine-2,4-dione (12.1) with melting point 111-114° C. Molecular weight 290.06 (C₁₂H₁₀F₄N₂O₂); retention time Rₜ=1.55 min. [B]; MS (ESI): 291.27 (MH⁺).

### 2) Preparation of 3-(4-fluoro-3-trifluoromethylphenyl)-5,5-dimethyl-1-(3-pentafluorosulfanylbenzyl)imidazolidine-2,4-dione (12)

The alkylating reaction (acetonitrile, cesium carbonate, 90 minutes, 70° C.) of 12.1 with 11.2 afforded 3-(4-fluoro-3-trifluoromethylphenyl)-5,5-dimethyl-1-(3-pentafluoro-sulfanylbenzyl)imidazolidine-2,4-dione (12; molecular weight 506.07 (C₁₉H₁₅F₉N₂O₂S); retention time Rₜ=2.24 min. [B]; MS (ESI): 507.15 (MH⁺)).

### EXAMPLE 13

### 4-[3-(3-chlorobenzyl)-4,4-dimethyl-2,5-dioxoimidazolidin-1-yl]-2-trifluoromethylbenzonitrile

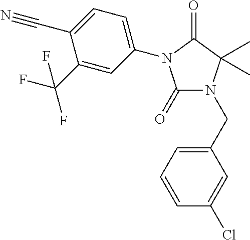 [0375] The alkylating reaction of compound 1.2 with 1-bromomethyl-3-chlorobenzene afforded 4-[3-(3-chlorobenzyl)-4,4-dimethyl-2,5-dioxoimidazolidin-1-yl]-2-trifluoromethylbenzonitrile 13. ¹H NMR (300 MHz): 8.33; d, 1H, 8.26; d, 1H, 8.10; dd, 1H, 7.53; t, 1H, 7.47-7.31; m, 3H, 4.62; s, 2H, 1.41; s, 6H.

### EXAMPLE 14

### 3-(4-fluoro-3-trifluoromethylphenyl)-5,5-dimethyl-1-(4-trifluoromethoxy-benzyl)imidazolidine-2,4-dione

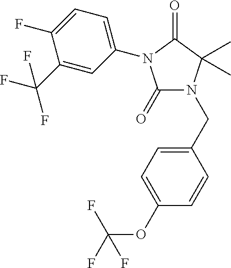 [0377] The compound of example 14 was prepared analogously to the procedure as described for the compound of example 7, by process “C”:

### 1) Preparation of methyl 2-methyl-2-{[1-(4-trifluoromethoxyphenyl)methylidene]-amino}propionate (14.1)

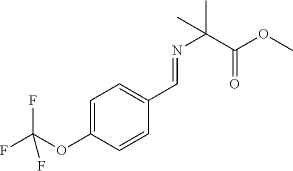 [0379] The reaction of methyl 2-amino-2-methylpropionate hydrochloride with 4-(trifluoromethoxy)benzaldehyde and triethylamine in dichloromethane afforded 14.1 (¹H NMR: 8.39; s, 1H, 7.9; d, 2H, 7.45; d, 2H, 3.68; s, 3H, 1.45; s, 6H).

### 2) Preparation of methyl 2-methyl-2-(4-trifluoromethoxybenzylamino)propionate (14.2)

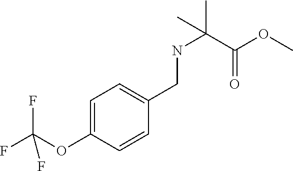 [0381] Compound 14.1 was dissolved in a mixture of dry dichloromethane and dry methanol, admixed with palladium-on-carbon (10%) and hydrogenated at 1 bar until the absorption of hydrogen had ended. This afforded methyl 2-methyl-2-(4-trifluoro-methoxybenzylamino)propionate (14.2, ¹H NMR: 7.43; d, 2H, 7.28; d, 2H, 3.63; s, 3H, 3.6; d, 2H, 2.51; t, 1H, 1.28; s, 6H).

### 3) Preparation of 3-(4-fluoro-3-trifluoromethylphenyl)-5,5-dimethyl-1-(4-trifluoro-methoxybenzyl)imidazolidine-2,4-dione

The amino acid ester 14.2 was dissolved in dry acetonitrile, admixed with 1-fluoro-4-isocyanato-2-trifluoromethylbenzene and stirred overnight at room temperature with exclusion of moisture. After the reaction had ended, the mixture was admixed with concentrated hydrochloric acid and stirred until complete ring closure for 3 h. This afforded 3-(4-fluoro-3-trifluoromethylphenyl)-5,5-dimethyl-1-(4-trifluoromethoxy-benzyl)imidazolidine-2,4-dione (14; ¹H NMR: 7.98; m, 1H, 7.88; m, 1H, 7.68; t, 1H, 7.58; d, 2H, 7.33; d, 2H, 4.63; s, 2H, 1.4; s, 6H).

### EXAMPLE 15

### 3-(4-chloro-3-trifluoromethylphenyl)-5,5-dimethyl-1-(4-trifluoromethoxy-benzyl)imidazolidine-2,4-dione

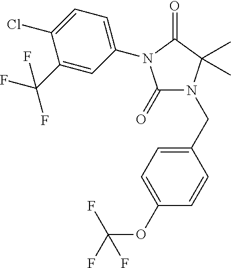 [0384] The compound of example 15 was prepared analogously to the procedure as described for the compound of example 14 by process “C”, except that, in the last stage, 1-chloro-4-isocyanato-2-trifluoromethylbenzene was used instead of 1-fluoro-4-isocyanato-2-trifluoromethylbenzene. 15: ¹H NMR: 8.05; s, 1H, 7.88; m, 2H, 7.58; d, 2H, 7.35; d, 2H, 4.62; s, 2H, 1.4; s, 6H.
The compounds of examples 19 (3-(3,4-difluorophenyl)-5,5-dimethyl-1-(4-trifluoromethoxy-benzyl)imidazolidine-2,4-dione,

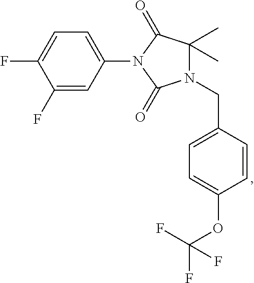

¹H NMR: 7.6; m, 4H, 7.35; m, 3H, 4.62; s, 2H, 1.39; s, 6H) and 20 (3-(3,4-dichlorophenyl)-5,5-dimethyl-1-(4-trifluoromethoxybenzyl)imidazolidine-2,4-dione,

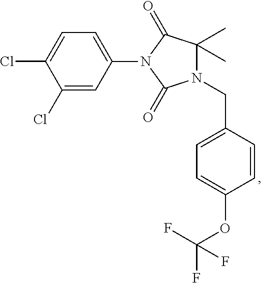

¹H NMR: 7.83; s, 1H, 7.79; d, 1H, 7.59; d, 2H, 7.51; d, 1H, 7.35; d, 2H, 4.62; s, 2H, 1.39; s, 6H) were obtained in an analogous manner by reacting 14.2 with, respectively, 1,2-difluoro-4-isocyanatobenzene and 1,2-dichloro-4-isocyanatobenzene.

### EXAMPLE 16

### 4-[4,4-dimethyl-2,5-dioxo-3-(2-trifluoromethylbenzyl)imidazolidin-1-yl]-2-trifluoromethylbenzonitrile

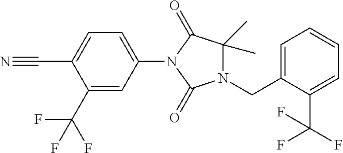 [0389] The compound of example 16 was obtained by reacting 1.2 with 1-bromomethyl-2-trifluoromethylbenzene. ¹H NMR: 8.36; d, 1H, 8.27; s, 1H, 8.11; m, 1H, 7.77; m, 2H, 7.69; t, 1H, 7.51; t, 1H, 4.74; s, 2H, 1.4; s, 6H.

The compounds of examples 25, 4-[3-(2-chlorobenzyl)-4,4-dimethyl-2,5-dioxoimidazolidin-1-yl]-2-trifluoromethylbenzonitrile,

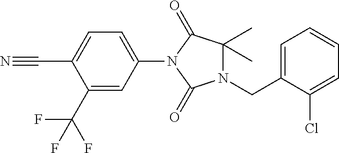

(¹H NMR: 8.35; d, 1H, 8.24; s, 1H, 8.1; d, 1H, 7.6; m, 1H, 7.49; m, 1H, 7.32; m, 2H, 4.68; s, 2H, 1.41; s, 6H); 26, 4-[3-(3,5-bis-(methanesulfonyl)benzyl)-4,4-dimethyl-2,5-dioxoimidazolidin-1-yl]-2-trifluoromethylbenzonitrile,

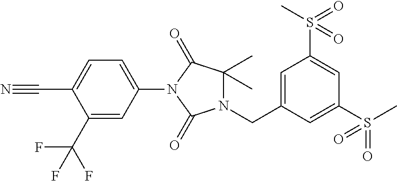

(¹H NMR: 8.36; m, 4H, 8.25; s, 1H, 8.1; d, 1H, 4.88; s, 2H, 2.55; s, 6H, 1.47; s, 6H); 27, 4-[3-(2-methanesulfonylbenzyl)-4,4-dimethyl-2,5-dioxoimidazolidin-1-yl]-2-trifluoromethylbenzonitrile,

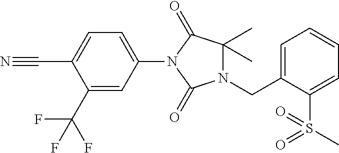

(¹H NMR: 8.36; d, 1H, 8.25; s, 1H, 8.1; d, 1H, 7.98; d, 1H, 7.78; d, 1H, 7.7; t, 1H, 7.59; t, 1H, 5.1; s, 2H, 2.55; s, 3H, 1.42; s, 6H); 28, 4-[3-(5-fluoro-2-methanesulfonylbenzyl)-4,4-dimethyl-2,5-dioxoimidazolidin-1-yl]-2-trifluoromethylbenzonitrile,

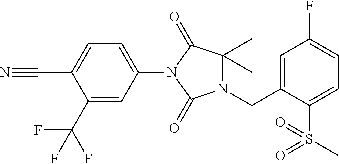

(¹H NMR: 8.35; d, 1H, 8.25; s, 1H, 8.1; d, 1H, 8.03; m, 1H, 7.62; d, 1H, 7.41; t, 1H, 5.06; s, 2H, 2.55; s, 3H, 1.48; s, 6H) and 29, 4-[3-(2-bromobenzyl)-4,4-dimethyl-2,5-dioxoimidazolidin-1-yl]-2-trifluoromethylbenzonitrile,

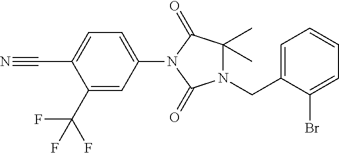

(¹H NMR: 8.35; d, 1H, 8.25; s, 1H, 8.1; d, 1H, 7.67; d, 1H, 7.59; d, 1H, 7.4; t, 1H, 7.26; t, 1H, 4.62; s, 2H, 1.42; s, 6H) were, as described for example 16, prepared by alkylating reaction of 1.2 with 1-bromomethyl-2-chlorobenzene (for 25), with 1-bromomethyl-3,5-bis(methanesulfonyl)benzene (for 26; prepared from the corresponding benzyl alcohol by reaction with phosphorus tribromide (¹H NMR: 8.39; m, 3H, 4.91; s, 2H, 2.55; s, 6H)), with 1-bromomethyl-2-methanesulfonylbenzene (for 27), with 2-bromomethyl-4-fluoro-1-methanesulfonylbenzene (for 28) and with 1-bromo-2-brommethylbenzene (for 29).

### EXAMPLE 17

### 1-(3,5-bis(trifluoromethyl)benzyl)-3-(3,4-difluorophenyl)-5,5-dimethyl-imidazolidine-2,4-dione

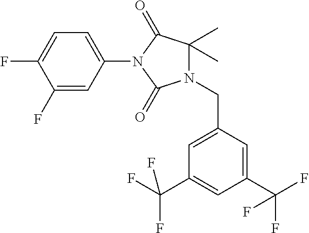 [0397] The compound of example 17 was prepared analogously to the procedure as described for the compound of example 7, by process “C”:
The reaction of 3,5-bis(trifluoromethyl)benzaldehyde with methyl 2-amino-2-methyl-propionate hydrochloride and triethylamine in dichloromethane afforded methyl 2-{[1-(3,5-bis(trifluoromethyl)phenyl)methylidene]amino}-2-methylpropionate (17.1; ¹H NMR: 8.59; s, 1H, 8.45; s, 2H, 8.25; s, 1H, 3.7; s, 3H), 1.5; s, 6H). The reduction of the imine with hydrogen and palladium on carbon afforded the amino acid ester derivative 17.2, methyl 2-(3,5-bis(trifluoromethyl)benzylamino)-2-methylpropionate (¹H NMR: 8.05; s, 2H, 7.94; s, 1H, 3.8; d, 2H, 3.61; s, 3H, 2.98; t, 1H, 1.28; s, 6H). The reaction of 17.2 with 1,2-difluoro-4-isocyanatobenzene afforded 1-(3,5-bis(trifluoromethyl)benzyl)-3-(3,4-difluorophenyl)-5,5-dimethylimidazolidine-2,4-dione (17; ¹H NMR: 8.16; s, 2H, 8.03; s, 1H, 7.62; m, 2H, 7.4; m, 1H, 4.8; s, 2H, 1.41; s, 6H).

The compound of example 18 (1-(3,5-bis(trifluoromethyl)benzyl)-3-(3,4-dichlorophenyl)-5,5-dimethyl-imidazolidine-2,4-dione

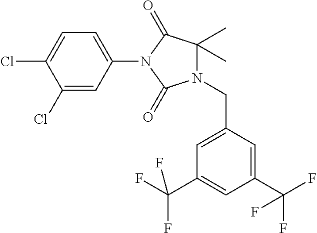

was prepared analogously by reacting 17.2 with 1,2-dichloro-4-isocyanatobenzene. ¹H NMR: 8.16; s, 2H, 8.03; s, 1H, 7.81; m, 2H, 7.53; d, 1H, 4.8; s, 2H, 1.4; s, 6H.

### EXAMPLE 23

### 3-(3,4-difluorophenyl)-5,5-dimethyl-1-(4-pentafluorosulfanylbenzyl)-imidazolidine-2,4-dione

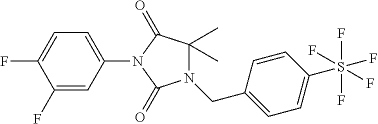 [0401] The compound of example 23 can be prepared by process “C”:

### 1) Preparation of N-methoxy-N-methyl-4-pentafluorosulfanylbenzamide (23.1)

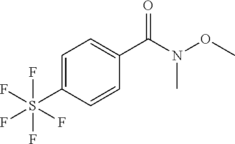 [0403] 1.25 g of 4-pentafluorosulfanylbenzoic acid and 0.54 g of N,O-dimethylhydroxylamine hydrochloride were dissolved in 20 ml of dichloromethane, and the solution was admixed with 3.2 g of 2,4,6-tripropyl-[1,3,5,2,4,6]trioxatriphosphinane 2,4,6-trioxide and 1.01 g of triethylamine and then stirred at room temperature for 16 h. For workup, the reaction mixture was concentrated under reduced pressure, and the residue was taken up in 50 ml of ethyl acetate and extracted by shaking twice with 25 ml each time of sodium hydrogensulfate solution and twice with 25 ml each time of saturated sodium carbonate solution. The organic phase was removed, dried over magnesium sulfate, filtered and concentrated under reduced pressure. This afforded N-methoxy-N-methyl-4-pentafluorosulfanylbenzamide (23.1), which was used in the next stage without further purification.

### 2) Preparation of 4-pentafluorosulfanylbenzaldehyde (23.2)

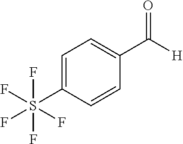 [0405] 1.31 g of compound 23.1 were dissolved in 35 ml of dry tetrahydrofuran and admixed dropwise at −60° C. while stirring with 4.95 ml of a 1 molar solution of lithium aluminum hydride in tetrahydrofuran. After the addition had ended, the mixture was stirred at −60° C. for another hour; then the mixture was allowed to warm to room temperature. For workup, the reaction mixture was admixed dropwise while stirring with 40 ml of cold potassium hydrogensulfate solution. The mixture was extracted by shaking twice with 50 ml each time of ethyl acetate; the organic phase was washed with saturated sodium chloride solution, dried over magnesium sulfate, filtered and concentrated under reduced pressure. This afforded 4-pentafluorosulfanyl-benzaldehyde (23.2). The aldehyde was processed further without further purification.

### 3) Preparation of methyl 2-methyl-2-{[1-(4-pentafluorosulfanylphenyl)methylidene]-amino}propionate (23.3)

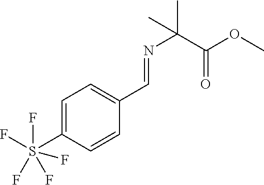 [0407] 0.53 g of methyl 2-amino-2-methylpropionate hydrochloride were suspended in 20 ml of dichloromethane and admixed dropwise while stirring with 0.35 g of triethylamine. 15 minutes after the addition had ended, the mixture was admixed with 0.83 g of magnesium sulfate and 0.8 g of compound 23.2 and stirred at room temperature for 24 hours. For workup, the reaction mixture was filtered, the filtrate was extracted by shaking with water and saturated sodium chloride solution, and the organic phase was removed, dried over magnesium sulfate, filtered and concentrated under reduced pressure. This afforded methyl 2-methyl-2-{[1-(4-pentafluorosulfanylphenyl)-methylidene]amino}propionate (23.3), which was reacted further as the crude product.

### 4) Preparation of methyl 2-methyl-2-(4-pentafluorosulfanylbenzylamino)propionate (23.4)

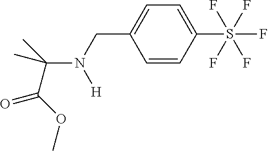 [0409] 1.05 g of compound 23.3 were dissolved in 20 ml of dry dichloromethane and admixed at room temperature with portions totaling 1.61 g of sodium triacetoxyborohydride. The mixture was stirred overnight at room temperature. For workup, the reaction mixture was admixed with 30 ml of saturated sodium hydrogencarbonate solution and 50 ml of dichloromethane. The organic phase was removed, extracted by shaking with saturated sodium chloride solution and dried over magnesium sulfate, filtered and concentrated under reduced pressure. The residue was purified by chromatography using silica gel with 3/1 n-heptane/ethyl acetate. This afforded methyl 2-methyl-2-(4-pentafluorosulfanylbenzylamino)propionate (23.4). ¹H NMR: 7.8; d, 2H, 7.56; d, 2H, 3.69; d, 2H, 3.62; s, 3H, 2.7; t, 1H, 1.29; s, 6H.

### 5) Preparation of 3-(3,4-difluorophenyl)-5,5-dimethyl-1-(4-pentafluorosulfanylbenzyl)-imidazolidine-2,4-dione

0.15 mmol of the amino acid ester 23.4 was dissolved in 1 ml of dry acetonitrile, admixed with 0.165 mmol of 1,2-difluoro-4-isocyanatobenzene and stirred overnight at room temperature with exclusion of moisture. After the reaction had ended, the mixture was admixed with 100 μl of concentrated hydrochloric acid and stirred until complete ring closure for 3 h. Thereafter, the solvent was removed under reduced pressure and the residue was purified by chromatography (method [RP1]). This afforded 3-(3,4-difluorophenyl)-5,5-dimethyl-1-(4-pentafluorosulfanylbenzyl)-imidazolidine-2,4-dione (23). ¹H NMR: 7.9; d, 2H, 7.62; m, 4H, 7.36; m, 1H, 4.7; s, 2H, 1.4; s, 6H.

### EXAMPLE 24

### 3-(3,4-dichlorophenyl)-5,5-dimethyl-1-(4-pentafluorosulfanyl-benzyl)imidazolidine-2,4-dione

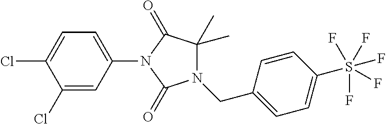 [0412] The compound of example 24 was obtained analogously by reacting 23.4 with 1,2-dichloro-4-isocyanatobenzene. ¹H NMR: 7.85; m, 4H, 7.67; d, 2H, 7.52; d, 1H, 4.7; s, 2H, 1.4; s, 6H.

Compound 32 (4-[3-(2-fluoro-3-methylbenzyl)-4,4-dimethyl-2,5-dioxoimidazolidin-1-yl]-2-trifluoromethylbenzonitrile,

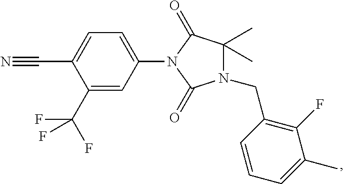

¹H NMR: 8.34; d, 1H, 8.21; s, 1H, 8.19; d, 1H, 7.35; t, 1H, 7.21; t, 1H, 7.09; t, 1H, 4.62; s, 2H, 2.25; s, 3H, 1.4; s, 6H).

was obtained in a manner analogous to that described for the compound of example 1, by reacting 1.2 with 1-bromomethyl-2-fluoro-3-methylbenzene.

### EXAMPLE 33

### 4-[3-(3,4-bis(benzyloxy)benzyl)-4,4-dimethyl-2,5-dioxoimidazolidin-1-yl]-2-trifluoromethylbenzonitrile

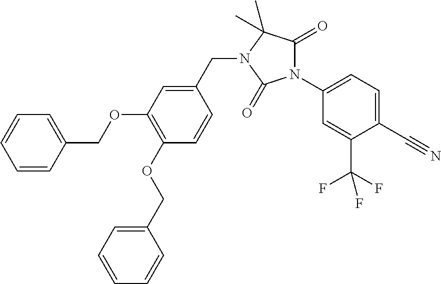

### 1) Preparation of methyl 2-(3,4-bis(benzyloxy)benzylamino)-2-methylpropionate 33.1

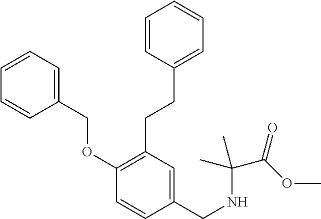 [0417] 10 g of methyl 2-amino-2-methylpropionate hydrochloride were suspended in 200 ml of dry dichloromethane, admixed dropwise while stirring with 6.587 g of triethylamine and, after the addition had ended, stirred for 15 minutes. Subsequently, 15.67 g of magnesium sulfate and 20.73 g of 3,4-dibenzyloxybenzaldehyde were added. The mixture was stirred at room temperature for 24 h. For workup, the suspension was filtered and the filtrate was extracted by shaking first with water and then with saturated sodium chloride solution. The organic phase was dried with magnesium sulfate and filtered, and the filtrate was concentrated under reduced pressure. This afforded 24.9 g of methyl 2-{[1-(3,4-bis(benzyloxy)phenyl)methylidene]amino}-2-methylpropionate 33.2. For further workup, the imine 33.2 was dissolved in 400 ml of dry dichloromethane, admixed with 31.6 g of sodium triacetoxyborohydride and stirred overnight at room temperature. For workup, the reaction mixture was admixed with sodium carbonate solution and dichloromethane; the organic phase was removed, dried over magnesium sulfate, filtered and concentrated under reduced pressure. The residue was purified by chromatography (silica gel; 2:1 n-heptane/ethyl acetate). This afforded methyl 2-(3,4-bis(benzyloxy)benzylamino)-2-methylpropionate 33.1. Molecular weight 419.20 (C₂₆H₂₉NO₄); retention time Rₜ=1.67 min. [C]; MS (ESI): 420.35 (MH⁺).

### 2) 4-[3-(3,4-bis(benzyloxy)benzyl)-4,4-dimethyl-2,5-dioxoimidazolidin-1-yl]-2-trifluoromethylbenzonitrile 33

0.165 mmol of 4-isocyanato-2-trifluoromethylbenzonitrile were added to a solution of 0.15 mmol of compound 33.1 in 1 ml of dry acetonitrile and the mixture was stirred overnight at room temperature. Subsequently, 100 μl of concentrated hydrochloric acid were added and the mixture was stirred for a further 3 h to complete the ring closure. The solvent was removed under reduced pressure and the residue was purified by chromatography (method RP1). This afforded 4-[3-(3,4-bis(benzyloxy)benzyl)-4,4-dimethyl-2,5-dioxoimidazolidin-1-yl]-2-trifluoromethylbenzonitrile: 33. ¹H NMR: 8.35; d, 1H, 8.24; s, 1H, 8.09; d, 1H, 7.47-7.27; m, 10H, 7.1; s, 1H, 7.0; m, 2H, 5.15; s, 2H, 5.11; s, 2H, 4.5; s, 2H, 1.3; s, 6H.

In an analogous manner (table 1), the compounds of examples 36, 37, 39 and 40 were prepared:

36 by reaction of 33.1 with 1-fluoro-4-isocyanato-2-trifluoromethylbenzene;
37 by reaction of 33.1 with 1-chloro-4-isocyanato-2-trifluoromethylbenzene;
39 by reaction of 33.1 with 1,2-difluoro-4-isocyanatobenzene;
and
40 by reaction of 33.1 with 1,2-dichloro-4-isocyanatobenzene.

TABLE 1

| | | | | | | Retention | HPLC- |
|---|---|---|---|---|---|---|---|
| Example | | Empirical | Molecular | | | time | MS |
| No. | Product | formula | weight | MH⁺ | ¹H NMR | T_{R} [min] | method |
| 36 | 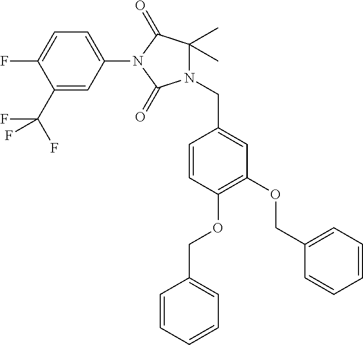 | C₃₃H₂₈F₄N₂O₄ | | | | | |
| 37 | 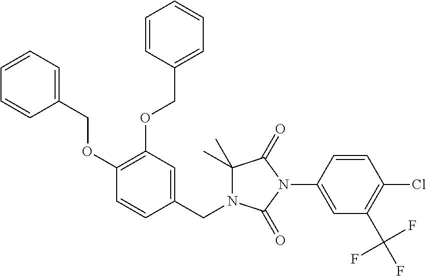 | C₃₃H₂₈ClF₃N₂O₄ | 608.16 | 609.43 | | 1.48 | B |
| 39 | 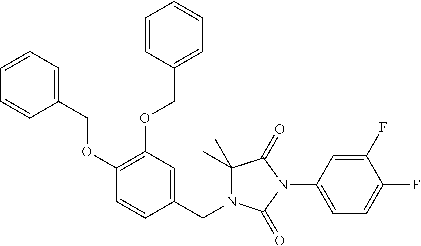 | C₃₂H₂₈F₂N₂O₄ | 542.20 | 543.50 | | 2.77 | B |
| 40 | 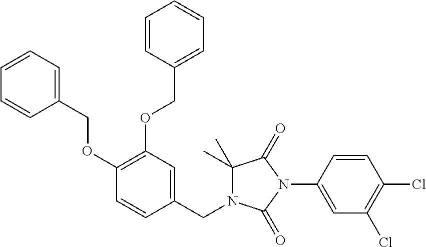 | C₃₂H₂₈Cl₂N₂O₄ | 574.14 | 575.36 | | 2.97 | B |

### EXAMPLE 34

### 4-[4,4-dimethyl-2,5-dioxo-3-(2-phenoxybenzyl)imidazolidin-1-yl]-2-trifluoromethylbenzonitrile

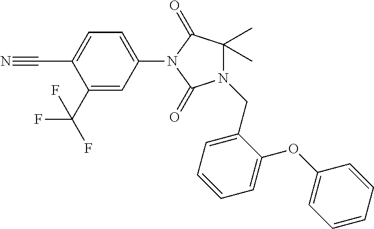

### 1) Preparation of 1-bromomethyl-2-phenoxybenzene (34.2)

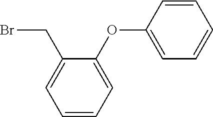 [0422] 2.5 g (12.5 mmol) of 2-phenoxybenzyl alcohol were dissolved in 45 ml of dichloromethane and admixed dropwise at 5° C. with a solution of 1.35 g (5 mmol) of phosphorus tribromide in 5 ml of dichloromethane. The mixture stood overnight at room temperature. Thereafter, the reaction mixture was admixed with 5 ml of saturated sodium carbonate solution, and the organic phase was removed, dried over magnesium sulfate, filtered and concentrated under reduced pressure. This afforded 3.25 g (quantitative) of 1-bromomethyl-2-phenoxybenzene 34.2. ¹H NMR: 7.56; d, 1H, 7.4; m, 2H, 7.3; m, 1H, 7.15; m, 2H, 7.01; d, 2H, 6.81; d, 1H, 4.7; s, 2H. Molecular weight 261.99 (C₁₃H₁₁BrO).

### 2) Preparation of tert-butyl 2-methyl-2-(2-phenoxybenzylamino)propionate (34.1)

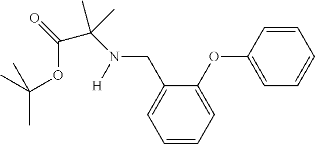 [0424] Compound 34.1 can be prepared by process “C”. For this purpose, 3.21 g (76.7 mmol) of lithium hydroxide hydrate were initially charged in 125 ml of dry dimethylformamide, admixed with 20 g of 4 A molecular sieve and stirred at room temperature for 30 minutes. Thereafter, 7.5 g (38.3 mmol) of tert-butyl 2-amino-2-methylpropionate hydrochloride were added and the mixture was stirred at room temperature for 15 minutes, before 11.09 g (42.16 mmol) of the bromide 34.2, dissolved in 25 ml of dry dimethylformamide, were added dropwise at room temperature. The reaction mixture was stirred at room temperature for 20 h. The reaction mixture was admixed with water and ethyl acetate, and the organic phase was removed, dried over magnesium sulfate, filtered and concentrated under reduced pressure. The residue was purified by chromatography (silica gel; 10/1 n-heptane/ethyl acetate) and afforded 8.3 g (64% yield) of tert-butyl 2-methyl-2-(2-phenoxy-benzylamino)propionate 34.1. Molecular weight 341.19 (C₂₁H₂₇NO₃); retention time Rₜ=1.58 min. [B]; MS (ESI): 342.49 (MH⁺).

### 3) 4-[4,4-Dimethyl-2,5-dioxo-3-(2-phenoxybenzyl)imidazolidin-1-yl]-2-trifluoromethyl-benzonitrile 34

As described in example 33, except using tert-butyl 2-methyl-2-(2-phenoxybenzyl-amino)propionate 33.1 and 4-isocyanato-2-trifluoromethylbenzonitrile, 34 was obtained. Molecular weight 479.15 (C₂₆H₂₀F₃N₃O₃); retention time Rₜ=2.93 min. [C]; MS (ESI): 480.28 (MH⁺).

The compounds of examples 43 (3-(3,4-dichlorophenyl)-5,5-dimethyl-1-(2-phenoxybenzyl)-imidazolidine-2,4-dione),

44 (3-(4-fluoro-3-trifluoromethylphenyl)-5,5-dimethyl-1-(2-phenoxybenzyl)imidazolidine-2,4-dione)
and 55 (3-(3,4-difluorophenyl)-5,5-dimethyl-1-(2-phenoxybenzyl)imidazolidine-2,4-dione) (see table 2)
were obtained in a manner similar to that described for the preparation of compound 34, by reacting 33.1 with 1,2-dichloro-4-isocyanatobenzene (for 43),
with 1-fluoro-4-isocyanato-2-trifluoromethylbenzene (for 44)
and with 1,2-difluoro-4-isocyanatobenzene (for 55).

TABLE 2

| | | | | | | Retention | HPLC- |
|---|---|---|---|---|---|---|---|
| Example | | Empirical | Molecular | | | time | MS |
| No. | Product | formula | weight | MH⁺ | ¹H NMR | T_{R} [min] | method |
| 43 | 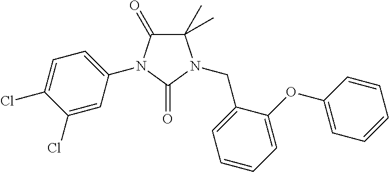 | C₂₄H₂₀Cl₂N₂O₃ | 454.09 | | 7.78, d, 2 H; 7.57, d, 1 H; 7.47, m, 1 H; 7.4, m, 2 H; 7.31, m, 1 H; 7.15, m, 2 H; 7.0, d, 2 H; 4.58, s, 2 H; 1.38, s, 6 H | | |
| 44 | 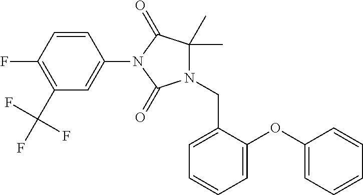 | C₂₅H₂₀F₄N₂O₃ | 472.14 | 473.57 | | 2.70 | C |
| 55 | 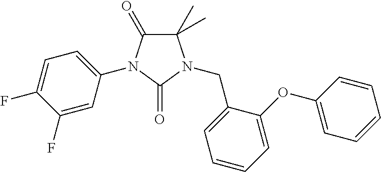 | C₂₄H₂₀F₂N₂O₃ | 422.14 | 423.15 | | 2.20 | B |

### EXAMPLE 35

### 4-(3-benzhydryl-4,4-dimethyl-2,5-dioxoimidazolidin-1-yl)-2-trifluoromethyl-benzonitrile

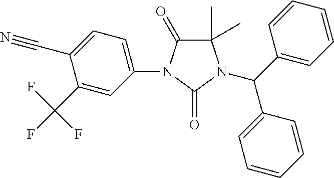 [0428] 100 mg of compound 1.2 were dissolved with 88 mg of bromodiphenylmethane in 2.5 ml of dry acetonitrile, admixed with 110 mg of cesium carbonate and stirred at room temperature for 4 h. For workup, the reaction mixture was admixed with ethyl acetate and water, and the organic phase was removed, dried over magnesium sulfate, filtered and concentrated under reduced pressure. The chromatographic purification was effected by method [RP2]. This afforded 4-(3-benzhydryl-4,4-dimethyl-2,5-dioxoimidazolidin-1-yl)-2-trifluoromethylbenzonitrile 35. ¹H NMR: 7.56; d, 1H, 7.4; m, 2H, 7.3; m, 1H, 7.15; m, 2H, 7.01; d, 2H, 6.81; d, 1H, 4.7; s, 2H.

The compounds of examples 38 (4-(3-biphenyl-2-ylmethyl-4,4-dimethyl-2,5-dioxo-imidazolidin-1-yl)-2-trifluoromethylbenzonitrile),
41 (4-[3-(2-isopropylbenzyl)-4,4-dimethyl-2,5-dioxoimidazolidin-1-yl]-2-trifluoromethyl-benzonitrile),
45 (3-[3-(4-cyano-3-trifluoromethylphenyl)-5,5-dimethyl-2,4-dioxoimidazolidin-1-ylmethyl]-benzoic acid),
46 (4-[3-(4-benzenesulfonylbenzyl)-4,4-dimethyl-2,5-dioxoimidazolidin-1-yl]-2-trifluoro-methylbenzonitrile),
48 (4-[4,4-dimethyl-2,5-dioxo-3-(4-phenoxybenzyl)imidazolidin-1-yl]-2-trifluoromethyl-benzonitrile),
49 (4-[3-(1,3-diphenylpropyl)-4,4-dimethyl-2,5-dioxoimidazolidin-1-yl]-2-trifluoromethyl-benzonitrile),
50 (4-[4,4-dimethyl-2,5-dioxo-3-(3-phenoxybenzyl)imidazolidin-1-yl]-2-trifluoromethyl-benzonitrile),
53 (4-(4,4-dimethyl-2,5-dioxo-3-pyridin-4-ylmethylimidazolidin-1-yl)-2-trifluoromethyl-benzonitrile) and
54 (4-[3-(3-hydroxybenzyl)-4,4-dimethyl-2,5-dioxoimidazolidin-1-yl]-2-trifluoromethyl-benzonitrile), (see table 3)
   were obtained in an analogous manner by reacting 1.2 with 2-bromomethylbiphenyl (for 38),
   with 1-bromomethyl-2-isopropylbenzene (41.1 for 41; 41.1 was obtained via the reaction sequence of methyl 2-isopropylbenzoate→(2-isopropylphenyl)methanol (41.2, by reduction with lithium aluminum hydride; ¹H NMR: 7.32; d, 1H, 7.27; d, 1H, 7.21; t, 1H, 7.15; t, 1H, 5.03; t, 1H, 4.55; d, 2H, 3.19; p, 1H, 1.19; d, 6H)→1-bromomethyl-2-isopropylbenzene (41.1, by reaction of 41.2 with phosphorus tribromide; ¹H NMR: 7.4-7.3; m, 3H, 7.18; t, 1H, 4.78; s, 2H, 3.3; p, 1H, 1.22; d, 6H)),
   with (2-bromoethoxymethyl)benzene (for 42; in this case, sodium hydride was used as the base and dimethylformamide as the solvent),
   with 1-benzenesulfonyl-4-brommethylbenzene (46.1 for 46; 46.1 (¹H NMR: 7.98; m, 4H, 7.7-7.6; m, 5H, 4.74; s, 2H) was obtained by reacting (4-benzenesulfonylphenyl)methanol (46.2; ¹H NMR: 7.93; m, 4H, 7.7-7.5; m, 5H, 5.41; t, 1H, 4.55; d, 2H) with phosphorus tribromide; the alcohol 46.2 had in turn been obtained by reduction of 4-benzenesulfonylbenzoic acid with lithium aluminum hydride),
   with 1-bromomethyl-4-phenoxybenzene (for 48),
   with 1-bromo-1,3-diphenylpropane (49.1 for 49; 49.1 was prepared from the corresponding alcohol by reaction with phosphorus tribromide: ¹H NMR: 7.6; m, 2H, 7.4-7.19; m, 8H, 5.2; t, 1H, 2.75; m, 1H, 2.55 m, 2H, 2.4; m, 1H),
   with 1-bromomethyl-3-phenoxybenzene (50.1 for 50; compound 50.1 was prepared by reaction of (3-phenoxyphenyl)methanol with phosphorus tribromide: ¹H NMR: 7.4; m, 3H, 7.2; m, 2H, 7.1; m, 1H, 7.03; m, 2H, 6.93; m, 1H, 4.7; s, 2H),
   with 4-bromomethylpyridine (for 53)
   and with (3-bromomethylphenoxy)trimethylsilane (with subsequent protecting group removal for 54).

TABLE 3

| | | | | | | Retention | HPLC/ |
|---|---|---|---|---|---|---|---|
| Example | | Empirical | Molecular | | | time | MS |
| No. | Product | formula | weight | MH⁺ | ¹H NMR | T_{R} [min] | method |
| 38 | 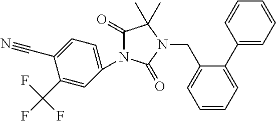 | C₂₆H₂₀F₃N₃O₂ | | | 8.31, d, 1 H; 8.18, s, 1 H; 8.02, d, 1 H; 7.58-7.22, m, 9 H; 4.55, s, 2 H; 1.19, s, 6 H | | |
| 41 | 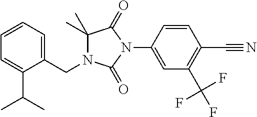 | C₂₃H₂₂F₃N₃O₂ | | | 8.35, d, 1 H; 8.23, s, 1 H; 8.1, d, 1 H; 7.39, d, 1 H; 7.33, d, 1 H; 7.28, t, 1 H; 7.18, t, 1 H; 4.69, s, 2 H; 3.3, p, 1 H; 1.38, s, 6 H; 1.22, d, 6 H | | |
| 45 | 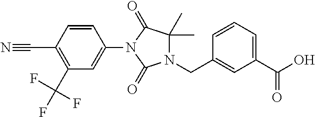 | C₂₁H₁₆F₃N₃O₄ | | | 13.0, s, broad, 1 H; 8.35, d, 1 H; 8.25, s, 1 H; 8.1, d, 1 H; 8.02, s, 1 H; 7.86, d, 1 H; 7.71, d, 1 H; 7.49, t, 1 H; 4.7, s, 2 H; 1.4, s, 6 H | | |
| 46 | 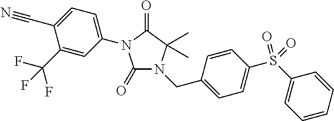 | C₂₆H₂₀F₃N₃O₄S | | | 8.34, d, 1 H; 8.21, s, 1 H; 8.08, d, 1 H; 7.97, m, 4 H; 7.2, m, 3 H; 7.61, m, 2 H: 4.7, s, 2 H; 1.4, s, 6 H | | |
| 48 | 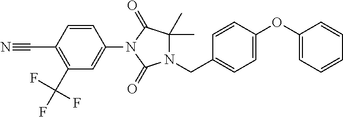 | C₂₆H₂₀F₃N₃O₃ | 479.15 | | 8.35, d, 1 H; 8.23, s, 1 H; 8.1, d, 1 H; 7.48, d, 2 H; 7.4, t, 2 H; 7.14, t, 1 H; 7.0, m, 4 H; 4.6, s, 2 H; 1.41, s, 6 H | | |
| 49 | 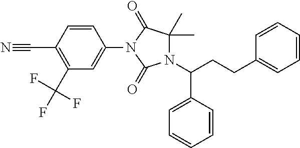 | C₂₈H₂₄F₃N₃O₂ | | | 8.34, d, 1 H; 8.22, s, 1 H; 8.09, d, 1 H; 7.61, d, 2 H; 7.4- 7.17, m, 8 H; 4.61, t, 1 H; 2.7, m, 2 H; 2.6, m, 1 H; 2.4, m, 1 H; 1.55, s, 3 H; 1.3, 8.3 H | | |
| 50 | 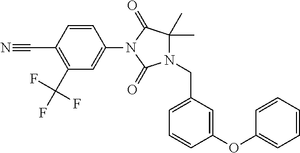 | C₂₆H₂₀F₃N₃O₃ | 479.15 | | 8.34, d, 1 H; 8.22, s, 1 H; 8.08, d, 1 H; 7.39, m, 3 H; 7.23, d, 1 H; 7.12, m, 2 H; 7.0, d, 2 H; 6.9, d, 1 H; 4.62, s, 2 H; 1.4, s, 6 H | | |
| 53 | 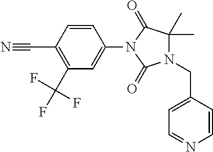 | C₁₉H₁₅F₃N₄O₂ | 388.35 | | 8.53, d, 2 H; 8.35, d, 1 H; 8.24, s, 1 H; 8.1, d, 1 H; 7.45, d, 2 H; 4.65, s, 2 H; 1.41, s, 6 H | | |
| 54 | 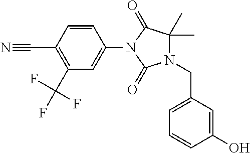 | C₂₀H₁₆F₃N₃O₃ | 403.36 | | 9.39, s, 1 H; 8.34, d, 1 H; 8.25, s, 1 H; 8.09, d, 1 H; 7.11, t, 1 H; 6.85, m, 2 H; 6.68, d, 1 H; 4.51, s, 2 H; 1.4, s, 6 H | | |

### EXAMPLE 51

### methyl 2-(3-{5-fluoro-2-[3-(4-fluoro-3-trifluoromethylphenyl)-5,5-dimethyl-2,4-dioxoimidazolidin-1-ylmethyl]phenyl}ureido)-2-methylpropionate

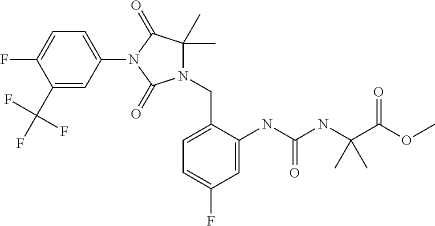

### 1) Preparation of 1-bromomethyl-4-fluoro-2-nitrobenzene (51.3)

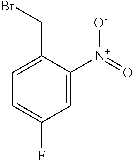 [0440] 0.776 g 4-fluoro-2-nitrotoluene was dissolved at room temperature in 10 ml of dry chlorobenzene and heated to 120° C. Subsequently, within 1 h, portions totaling 1.07 g of N-bromosuccinimide and 0.12 g of benzoyl peroxide were added mixed thoroughly. After the addition had ended, the mixture was stirred at 120° C. for a further hour. For workup, the cooled reaction mixture was concentrated under reduced pressure and the residue was taken up with methyl tert-butyl ether. The ethereal solution was washed with 1 N sodium hydroxide solution and then with saturated sodium chloride solution, and the organic phase was dried over magnesium sulfate, filtered and concentrated under reduced pressure. After chromatographic purification (silica gel; 90/10 n-heptane/ethyl acetate 80/20 n-heptane/ethyl acetate in 35 min.), 1-bromomethyl-4-fluoro-2-nitrobenzene (51.3) was obtained. ¹H NMR: 8.01; d, 1H, 7.83; t, 1H, 7.69; t, 1H, 4.9; s, 2H.

### 2) Preparation of 1-(4-fluoro-2-nitrobenzyl)-3-(4-fluoro-3-trifluoromethylphenyl)-5,5-dimethylimidazolidine-2,4-dione (51.2)

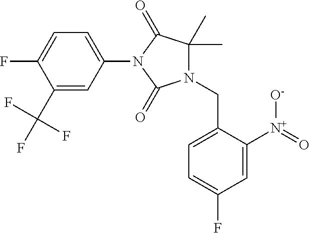 [0442] 0.73 g of the compound of example 47 was dissolved at room temperature in 20 ml of dry acetonitrile, admixed with 0.7 g of compound 51.3 and 0.9 g of cesium carbonate and stirred at room temperature for 24 h. For workup, the reaction mixture was filtered; the filtrate was concentrated under reduced pressure, and the residue was stirred with water, filtered off with suction, washed with water and dried. This afforded 1-(4-fluoro-2-nitrobenzyl)-3-(4-fluoro-3-trifluoromethylphenyl)-5,5-dimethylimidazolidine-2,4-dione (51.2). Molecular weight 443.09 (C₁₉H₁₄F₅N₃O₄); retention time Rₜ=3.56 min. [D]; MS (ESI): 444.08 (MH⁺).

### 3) Preparation of 1-(2-amino-4-fluorobenzyl)-3-(4-fluoro-3-trifluoromethylphenyl)-5,5-dimethylimidazolidine-2,4-dione (51.1)

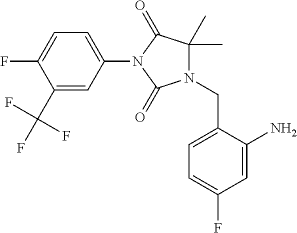 [0444] 1.03 g of compound 51.2 were dissolved at room temperature in 20 ml of dry methanol and, under an argon atmosphere, 16 mg of palladium hydroxide-on-carbon and then 0.20 g of trimethylamine-borane complex were added and the reaction mixture was stirred under reflux for 4 h. After addition of a further 0.1 g of trimethylamine-borane complex and heating for another 4 hours, the reaction had ended. For workup, the cooled reaction mixture was filtered through a fluted filter, the filtrate was concentrated under reduced pressure and the residue was stirred with cyclohexane, filtered off with suction, washed with cyclohexane and dried. This afforded 1-(2-amino-4-fluorobenzyl)-3-(4-fluoro-3-trifluoromethylphenyl)-5,5-dimethylimidazolidine-2,4-dione (51.1). Molecular weight 413.11 (C₁₉H₁₆F₅N₃O₂); retention time Rₜ=3.78 min. [E]; MS (ESI): 414.06 (MH⁺).

### 4) Methyl 2-(3-{5-fluoro-2-[3-(4-fluoro-3-trifluoromethylphenyl)-5,5-dimethyl-2,4-dioxoimidazolidin-1-ylmethyl]phenyl}ureido)-2-methylpropionate

0.21 g of the compound of example 51.1 was dissolved at room temperature in 5 ml of dry pyridine, supplied with 0.14 g of methyl 2-isocyanoto-2-methylpropionate and stirred at room temperature for 24 h. For workup, the reaction mixture was concentrated under reduced pressure and the residue was purified chromatographically (method [RP1]). This afforded methyl 2-(3-{5-fluoro-2-[3-(4-fluoro-3-trifluoro-methylphenyl)-5,5-dimethyl-2,4-dioxoimidazolidin-1-ylmethyl]phenyl}ureido)-2-methylpropionate 51. ¹H NMR: 8.1; s, 1H, 8.9; m, 1H, 7.9; m, 1H, 7.7; t, 1H, 7.54; d, 1H, 7.4; t, 1H, 7.0; s, 1H, 6.82; t, 1H, 4.48; s, 2H, 3.6; s, 3H, 1.45; s, 3H, 1.4; s, 3H.

### EXAMPLE 52

### N-{5-fluoro-2-[3-(4-fluoro-3-trifluoromethylphenyl)-5,5-dimethyl-2,4-dioxoimidazolidin-1-ylmethyl]phenyl}sulfamide

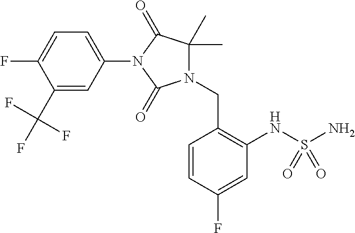

### 1) Preparation of N-{5-fluoro-2-[3-(4-fluoro-3-trifluoromethylphenyl)-5,5-dimethyl-2,4-dioxoimidazolidin-1-ylmethyl]phenyl}N′-tert-butyloxycarbonylsulfamide (52.1)

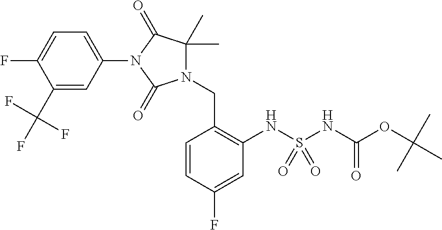 [0448] 0.35 g of compound 51.1 was dissolved at room temperature in 15 ml of dry dichloromethane, admixed with 0.28 g of N-(tert-butoxycarbonyl)sulfamoyl chloride (prepared from chlorosulfonyl isocyanate and tert-butanol; A. Casini et al., Bioorg. Med. Chem. Lett. 13 (2003) 837-840) and 0.18 ml of triethylamine, and stirred at room temperature for 4 h. For workup, the reaction mixture was concentrated under reduced pressure, and the residue was stirred with water, filtered off with suction, washed with water and dried. This afforded N-{5-fluoro-2-[3-(4-fluoro-3-trifluoro-methylphenyl)-5,5-dimethyl-2,4-dioxoimidazolidin-1-ylmethyl]phenyl}-N′-tert-butyl-oxycarbonylsulfamide 52.1. Molecular weight 592.14 (C₂₄H₂₅F₅N₄O₆S); retention time Rₜ=3.68 min. [B]; MS (ESI): 537.05 (MH⁺—C₄H₈).

### 2) N-{5-Fluoro-2-[3-(4-fluoro-3-trifluoromethylphenyl)-5,5-dimethyl-2,4-dioxoimidazolidin-1-ylmethyl]phenyl}sulfamide

0.69 g of compound 52.1 was dissolved at room temperature in 15 ml of dry dichloromethane, admixed with 1.79 ml of trifluoroacetic acid and 0.18 ml of water, and stirred at room temperature for 4 h and then left to stand overnight. For workup, the reaction mixture was concentrated under reduced pressure, and the residue was admixed with toluene and concentrated again. Finally, the residue was dissolved in dichloromethane, the organic phase was washed with saturated sodium hydrogencarbonate solution, dried over magnesium sulfate and filtered, and the filtrate was concentrated under reduced pressure. This afforded N-{5-fluoro-2-[3-(4-fluoro-3-trifluoromethylphenyl)-5,5-dimethyl-2,4-dioxoimidazolidin-1-ylmethyl]phenyl}-sulfamide 52. Molecular weight 492.08 (C₁₉H₁₇F₅N₄O₆S); retention time Rₜ=3.24 min. [D]; MS (ESI): 493.10 (MH⁺).

The inventive compounds of the formula I exhibit a high affinity for the human cannabinoid receptor 1 (hCB1R). This affinity is significantly more marked compared to that on the human androgen receptor (hAR). For instance, the selectivity is greater by about a factor of 5 than was found for examples of the compounds described in application U.S. Pat. No. 5,411,981.

Pharmacological Tests:

Binding to Human Cannabinoid Receptor 1 (hCB1R):

Test compounds: The compounds (3 μl, 10 mM, 100% DMSO), pipetted into 96-well PP microtiter plates, were diluted with 27 μl of 100% DMSO (dimethyl sulfoxide). Proceeding from this solution, further 3-fold dilution steps were undertaken by transferring 10 μA in each case to a new PP microtiter plate and adding a further 20 μl of 100% DMSO. In each case 6 μl of these solutions were transferred into new 96-well PP microtiter plates and made up with 144 μl of assay buffer. The end concentrations ranged from 10 μM to 0.005 μM.

Negative control: AM 251, dissolved in assay buffer with 1% DMSO, was added to the dilution series in the microtiter plates as a control. The end concentration was 1 μM.

Blank control: assay buffer with 1% DMSO was added to the dilution series of the microtiter plates as a blank control.

Summary of the Assay Parameters:

| | | |
|---|---|---|
| Assay volume | | 200 μl |
| Receptor | CHO-K1/cannabinoid CB1 | 2 μg/well |
| | Protein | |
| Ligand | [³H]-SR141716A | 0.5 nM |
| | | 0.0195 μCi/well |
| Ions | Tris-HCl | 50 mM, pH 7.4 |
| | MgCl₂ | 5 mM |
| | EDTA | 2.5 mM |
| | BSA (fatty acid-free) | 0.2% |
| Nonspecific binding | AM 251 | 1 μM |
| Compound | in 1% DMSO | 10 μM to 0.0050 μM |

Analysis of the Data:

High control: ³H binding without addition of the compound

Low control: ³H binding in the presence of 1 μM AM 251

The values were calculated using the corrected raw data.

$$Inhibition of ligand binding ( \% ) = 100 * ( 1 - \frac{( sample - lowcontrol )}{( highcontrol - lowcontrol )} )$$

The values reported were obtained as average values of a double determination. The IC₅₀ values were calculated from the measurements with the program Xlfit, formula 205. Ki values were obtained from the IC₅₀ and Kd values utilizing the Cheng-Prusoff equation:

$$Ki = \frac{IC 50}{1 + \frac{C}{Kd}}$$

$$( C = concentration of the radioligand )$$

Literature: Cheng, Y.-C., and Prusoff, W. H. (1973) Biochem. Pharmacol 22, 3099-3108

Results: Kᵢ values of example compounds; table 4:

| Example No. | hCB1R; binding Kᵢ [nM] |
|---|---|
| 1 | 4 |
| 2 | 200 |
| 3 | 127 |
| 5 | 25 |
| 6 | 11 |
| 7 | 205 |
| 8 | 227 |
| 9 | 295 |
| 10 | 81 |
| 11 | 17 |
| 33 | 26 |
| 34 | 6 |
| 37 | 219 |
| 44 | 100 |
| 52 | 41 |

It can be seen from the test data that the inventive compounds of the formula I bind with high affinity to hCB1R and are therefore very suitable for treatment of metabolic syndrome, of type II diabetes and of obesity.

Binding Assay with Human Androgen Receptor:

The binding assays on the androgen receptor were conducted according to the method of D. T. Zava et al. (1979) (“Androgen Receptor Assay with [³H]Methyltrienolone (R1881) in the Presence of Progesterone Receptor”, Endocrinology, 104, 1007-1012). The radioactive ligand used for the binding measurement was [³H]methyltrienolone and the reference substance was the same compound in unmarked (=nonradioactive) form. To determine the unspecific proportion of binding, 1 μM mibolerone was used. In a departure from the method cited, for the preparation of cytosolic receptor protein, the androgen-sensitive human prostate adenocarcinoma cell line LNCaP was used. For the analysis, aliquots of a cell cytosol fraction (proceeding from 10⁶ cells per analysis point) were incubated, in a buffer in the presence or absence of test substance, with 0.5 nM [³H]methyltrienolone at 4° C. for 24 hours (25 mM HEPES/Tris, 1 mM EDTA, 10 mM Na₂MoO₄, 2 mM DTT, 10% glycerol; pH 7.4). The samples were then mixed with 400 μl each of an activated carbon suspension and the mixtures were centrifuged (10 minutes, 8000×g). Supernatants were withdrawn and mixed with 5 ml each of scintillation cocktail, and the radioactivity of the samples was measured in a scintillation counter. Specific ligand binding to the receptors was calculated as the difference between total binding and the unspecific binding in the presence of an excess of nonmarked ligand. End results were represented as percent specific binding compared to the binding of control substance.

The strength of the binding to the human androgen receptor is expressed as percent inhibition of the binding of [³H]methyltrienolone to the human androgen receptor. The concentration of the compounds examined is 1 μM or 10 μM. The greater the numerical value of “percent inhibition at 1 or 10 μM”, the stronger is the binding of the test substance to the human androgen receptor. Alternatively, the Ki value is reported; the greater this value compared to the Ki value based on the binding to the hCB1R, the lower is the binding to the hAR.

Results: percent inhibition of the binding of [³H]methyltrienolone to the human androgen receptor (hAR) by example compounds at 1 or 10 μM; table 5:

| | hAR; % inhibition |
|---|---|
| Example No. | at 1/10 μM |
| 1 | 26% (10 μM) |
| 2 | 20% (1 μM) |
| 3 | 54% (1 μM) |
| 5 | 86% (1 μM) |
| 6 | 84% (1 μM) |
| 7 | 47% (1 μM) |
| 8 | 53% (1 μM) |
| 9 | 3% (10 μM) |
| 10 | 3% (1 μM) |
| 11 | 39% (1 μM) |
| 33 | Ki > 1 μM |
| 34 | 30% (1 μM) |
| 37 | 4% (10 μM) |
| 44 | 15% (10 μM) |
| 52 | 52% (1 μM) |

The compound of example 29 (4-[4,4-dimethyl-2,5-dioxo-3-(4-trifluoromethylbenzyl)-imidazolidin-1-yl]-2-trifluoromethylbenzonitrile) of application U.S. Pat. No. 5,411,981 has a Kᵢ value of 76 nM based on its binding to human cannabinoid receptor 1 and a value of 96 nM based on its binding to the human androgen receptor.

It can be seen from the test data of tables 4 and 5 that the inventive compounds of the formula I have a significantly or very significantly reduced affinity with respect to the human androgen receptor, and the selectivity with respect to human cannabinoid receptor 1 is increased.

1. A compound of the formula I 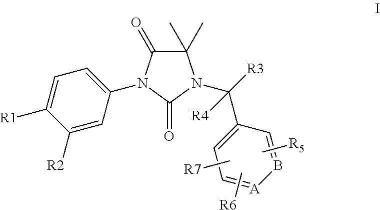
in which
R1 is CN, NO₂ or halogen;
R2 is CF₃ or halogen;
A, B are each independently CH, N;
R3, R4 are each independently hydrogen, (C₁-C₁₂)-alkyl, ((C₆-C₁₂)-aryl, (C₁-C₁₂)-alkylene-(C₆-C₁₂)-aryl, where (C₁-C₁₂)-alkyl, (C₆-C₁₂)-aryl, (C₁-C₁₂)-alkylene-(C₆-C₁₂)-aryl may each be up to trisubstituted independently by halogen, CN, CF₃;
R5, R6, R7 are each independently H, F, Cl, Br, CN, CF₃, SF₅, OCF₃, NO₂, S(O)ₘ[(C₁-C₆)-alkyl], S(O)ₘ[(C₃-C₉)-cycloalkyl], S(O)ₘCF₃, (C₁-C₆)-alkyl, (C₁-C₆)-alkoxy, (C₂-C₆)-alkenyl, (C₂-C₆)-alkenyloxy, (C₂-C₆)-alkynyl, (C₂-C₆)-alkynyloxy, OH, SH, W—COO—[(C₁-C₁₂)-alkyl], —O(C═O)—(C₆-C₁₂)-aryl, W—COOH, W—CONH₂, W—CO—NH[(C₁-C₆)-alkyl],
W—CO—N[(C₁-C₆)-alkyl]₂, W—CO—NH[(C₃-C₉)-cycloalkyl], W—CO—N[(C₃-C₉)-cycloalkyl]₂, W—CO—NH—CN, W—CO—NH—CHR8-CO—R9 W—CO—R10, W—CO—NH—C(═NH)NH₂, W—CO—NH—C(═NH)NH[(C₁-C₆)-alkyl], W—CO—NH—C(═NH)N[(C₁-C₆)-alkyl]₂, (C₁-C₈)-acyl, (C₁-C₇)-acyloxy, W—C(═NH)NH₂, W—C(═NH)NHOH, W—C(═N—SO₂—NH₂)NH₂, W—C(═N—SO₂—CF₃)NH₂, W—C[═N—SO₂—(C₁-C₆)-alkyl]NH₂, W—C[═N—SO₂—(C₃-C₉)-cycloalkyl]NH₂, W—C(═N—SO₂-aryl)NH₂, NH₂, NH—(C₁-C₁₂)-alkyl, N—[(C₁-C₁₂)-alkyl]₂, W—NH—C(═NH)NH₂, W—NH—C(═NH)NH[(C₁-C₆)-alkyl], W—NH—C(═NH)N[(C₁-C₆)-alkyl]₂, W—NH—CO—NH₂, W—NH—CO—NH[(C₁-C₆)-alkyl], W—NH—CO—N[(C₁-C₆)-alkyl]₂, W—NH—CO—NH[(C₃-C₉)-cycloalkyl], W—NH—CO—N[(C₃-C₉)-cycloalkyl]₂, W—NH—CO—NH—[(C₁-C₆)-alkyl]-CO—O—[(C₁-C₆)-alkyl], W—NH—CO—NH—[(C₁-C₆)-alkyl]-CO—NH₂, W—NH—CO—NH—SO₂—(C₁-C₆)-alkyl, W—NH—CO—NH—SO₂—[(C₃-C₉)-cycloalkyl], W—NH—CO—NH—CO—(C₁-C₆)-alkyl, W—NH—CO—NH—CO—[(C₃-C₉)-cycloalkyl], W—NH—C(═NH)—NH—C(═NH)—NH₂, W—NH—C(═NH)—NH—C(═NH)—NH[(C₁-C₆)-alkyl], W—NH—C(═NH)—NH—C(═NH)—N[(C₁-C₆)-alkyl]₂, W—NH—W—SO₂—NH₂, W—NH—W—SO₂—NH[(C₁-C₆)-alkyl], W—NH—W—SO₂—N[(C₁-C₆)-alkyl]₂, W—NH—W—SO₂—NH[(C₃-C₉)-cycloalkyl], W—NH—W—SO₂—N[(C₃-C₉)-cycloalkyl]₂, W—NH—W—SO₂—NH—CO—O[(C₁-C₆)-alkyl], W—NH—W—SO₂—NH—CO—NH₂, W—O—SO₂—NH₂, W—O—W—COOH, W—O—W—CONH₂, W—SO₂—NH₂, W—SO₂—NH[(C₁-C₆)-alkyl], W—SO₂—N[(C₁-C₆)-alkyl]₂, W—SO₂—NH[(C₃-C₉)-cycloalkyl], W—SO₂—N[(C₃-C₉)-cycloalkyl]₂, W—SO₃H, W—NH—W—SO₃H, W—SO₂—NH—CO—NH₂, W—SO₂—NH—CO—NH[(C₁-C₆)-alkyl], W—SO₂—NH—CO—N[(C₁-C₆)-alkyl]₂, W—SO₂—NH—CO—NH[(C₃-C₉)-cycloalkyl], W—SO₂—NH—CO—N[(C₃-C₉)-cycloalkyl]₂, W—P(O)(OH)[O—(C₁-C₆)-alkyl], W—P(O)[O—(C₁-C₆)-alkyl]₂, W—P(O)(OH)(O—CH₂-aryl), W—P(O)(O—CH₂-aryl)₂, W—P(O)(OH)₂, (C₆-C₁₂)-aryl, O—(C₆-C₁₂)-aryl, O—(C₁-C₁₂)-alkylene-(C₆-C₁₂)-aryl, S(O)ₘ—(C₆-C₁₂)-aryl, tri(C₁-C₁₂)-alkylsilyl, where the alkyl has from 1 to 6 carbon atoms;
m is 0, 1, 2;
W is a bond or (C₁-C₆)-alkyl;
R8 is H, (C₁-C₆)-alkyl, where the alkyl group may be substituted by OH, SH, SCH₃, aryl, 4-hydroxyaryl, heteroaryl, NH₂, NH—C(═NH)NH₂, COOH, CO—O(C₁-C₆)-alkyl, CONH₂;
R9 is OH, NH₂, NH—(C₁-C₁₂)-alkyl, N[(C₁-C₁₂)-alkyl]₂, NH—(C₃-C₉)-cycloalkyl, N[(C₃-C₉)-cycloalkyl]₂;
R10 is NH—(C₁-C₆)-alkyl-SO₃H, NH—(C₁-C₆)-alkyl-SO₂NH₂, NH—(C₁-C₆)-alkyl-SO₂—(C₁-C₆)-alkyl, NH—(C₁-C₆)-alkyl-SO₂—(C₃-C₉)-cycloalkyl, NH—(C₁-C₆)-alkyl-SO₂—CF₃, 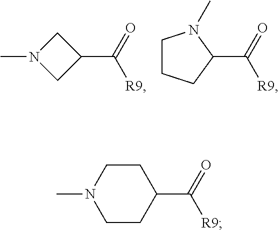
and the physiologically compatible salts thereof.

2. The compound of claim 1, wherein,
R1 is CN or halogen;
R2 is CF₃ or halogen;
A, B are each independently CH, N;
R3, R4 are each independently hydrogen, (C₁-C₁₂)-alkyl, ((C₆-C₁₂)-aryl, (C₁-C₁₂)-alkylene-(C₆-C₁₂)-aryl, where (C₁-C₁₂)-alkyl, (C₆-C₁₂)-aryl, (C₁-C₁₂)-alkylene-(C₆-C₁₂)-aryl may each be up to trisubstituted independently by halogen, CN, CF₃;
R5 F, Cl, Br, CN, CF₃, SF₅, OCF₃, NO₂, S(O)ₘ[(C₁-C₆)-alkyl], S(O)ₘ[(C₃-C₉)-cycloalkyl], S(O)ₘCF₃, (C₁-C₆)-alkyl, (C₁-C₆)-alkoxy, (C₂-C₆)-alkenyl, (C₂-C₆)-alkenyloxy,
(C₂-C₆)-alkynyl, (C₂-C₆)-alkynyloxy, OH, SH, W—COO—[(C₁-C₁₂)-alkyl], —O(C═O)—(C₆-C₁₂)-aryl, W—COOH, W—CONH₂, W—CO—NH[(C₁-C₆)-alkyl], W—CO—N[(C₁-C₆)-alkyl]₂, W—CO—NH[(C₃-C₉)-cycloalkyl], W—CO—N[(C₃-C₉)-cycloalkyl]₂, W—CO—NH—CN, W—CO—NH—CHR8-CO—R9, W—CO—R10, W—CO—NH—C(═NH)NH₂, W—CO—NH—C(═NH)NH[(C₁-C₆)-alkyl], W—CO—NH—C(═NH)N[(C₁-C₆)-alkyl]₂, (C₁-C₈)-acyl, (C₁-C₇)-acyloxy, W—C(═NH)NH₂, W—C(═NH)NHOH, W—C(═N—SO₂—NH₂)NH₂, W—C(═N—SO₂—CF₃)NH₂, W—C[═N—SO₂—(C₁-C₆)-alkyl]NH₂, W—C[═N—SO₂—(C₃-C₉)-cycloalkyl]NH₂, W—C(═N—SO₂-aryl)NH₂, NH₂, NH—(C₁-C₁₂)-alkyl, N—[(C₁-C₁₂)-alkyl]₂, W—NH—C(═NH)NH₂, W—NH—C(═NH)NH[(C₁-C₆)-alkyl], W—NH—C(═NH)N[(C₁-C₆)-alkyl]₂, W—NH—CO—NH₂, W—NH—CO—NH[(C₁-C₆)-alkyl], W—NH—CO—N[(C₁-C₆)-alkyl]₂, W—NH—CO—NH[(C₃-C₉)-cycloalkyl], W—NH—CO—N[(C₃-C₉)-cycloalkyl]₂, W—NH—CO—NH—[(C₁-C₆)-alkyl]-CO—O—[(C₁-C₆)-alkyl], W—NH—CO—NH—[(C₁-C₆)-alkyl]-CO—NH₂, W—NH—CO—NH—SO₂—(C₁-C₆)-alkyl, W—NH—CO—NH—SO₂—[(C₃-C₉)-cycloalkyl], W—NH—CO—NH—CO—(C₁-C₆)-alkyl, W—NH—CO—NH—CO—[(C₃-C₉)-cycloalkyl], W—NH—C(═NH)—NH—C(═NH)—NH₂, W—NH—C(═NH)—NH—C(═NH)—NH[(C₁-C₆)-alkyl], W—NH—C(═NH)—NH—C(═NH)—N[(C₁-C₆)-alkyl]₂, W—NH—W—SO₂—NH₂, W—NH—W—SO₂—NH[(C₁-C₆)-alkyl], W—NH—W—SO₂—N[(C₁-C₆)-alkyl]₂, W—NH—W—SO₂—NH[(C₃-C₉)-cycloalkyl], W—NH—W—SO₂—N[(C₃-C₉)-cycloalkyl]₂, W—NH—W—SO₂—NH—CO—O[(C₁-C₆)-alkyl], W—NH—W—SO₂—NH—CO—NH₂, W—O—SO₂—NH₂, W—O—W—COOH, W—O—W—CONH₂, W—SO₂—NH₂, W—SO₂—NH[(C₁-C₆)-alkyl], W—SO₂—N[(C₁-C₆)-alkyl]₂, W—SO₂—NH[(C₃-C₉)-cycloalkyl], W—SO₂—N[(C₃-C₉)-cycloalkyl]₂, W—SO₃H, W—NH—W—SO₃H, W—SO₂—NH—CO—NH₂, W—SO₂—NH—CO—NH[(C₁-C₆)-alkyl], W—SO₂—NH—CO—N[(C₁-C₆)-alkyl]₂, W—SO₂—NH—CO—NH[(C₃-C₉)-cycloalkyl], W—SO₂—NH—CO—N[(C₃-C₉)-cycloalkyl]₂, W—P(O)(OH)[O—(C₁-C₆)-alkyl], W—P(O)[O—(C₁-C₆)-alkyl]₂, W—P(O)(OH)(O—CH₂-aryl), W—P(O)(O—CH₂-aryl)₂, W—P(O)(OH)₂, (C₆-C₁₂)-aryl, O—(C₆-C₁₂)-aryl, O—(C₁-C₁₂)-alkylene-(C₆-C₁₂)-aryl, S(O)ₘ—(C₆-C₁₂)-aryl, tri(C₁-C₁₂)-alkylsilyl, where the alkyl has from 1 to 6 carbon atoms;
R6, R7 are each independently H, halogen, CN, CF₃, SF₅, OCF₃, S(O)ₘ[(C₁-C₆)-alkyl], S(O)ₘ[(C₃-C₉)-cycloalkyl], NO₂, S(O)ₘCF₃, (C₁-C₆)-alkyl, (C₁-C₆)-alkoxy, (C₂-C₆)-alkenyl, (C₂-C₆)-alkenyloxy,
(C₂-C₆)-alkynyl, (C₂-C₆)-alkynyloxy, OH, SH, W—COO—[(C₁-C₁₂)-alkyl], —O(C═O)—(C₆-C₁₂)-aryl, W—COOH, W—CONH₂, W—CO—NH[(C₁-C₆)-alkyl], W—CO—N[(C₁-C₆)-alkyl]₂, W—CO—NH[(C₃-C₉)-cycloalkyl], W—CO—N[(C₃-C₉)-cycloalkyl]₂, W—CO—NH—CN, W—CO—NH—CHR8-CO—R9, W—CO—R10, W—CO—NH—C(═NH)NH₂, W—CO—NH—C(═NH)NH[(C₁-C₆)-alkyl], W—CO—NH—C(═NH)N[(C₁-C₆)-alkyl]₂, (C₁-C₈)-acyl, (C₁-C₇)-acyloxy, W—C(═NH)NH₂, W—C(═NH)NHOH, W—C(═N—SO₂—NH₂)NH₂, W—C(═N—SO₂—CF₃)NH₂, W—C[═N—SO₂—(C₁-C₆)-alkyl]NH₂, W—C[═N—SO₂—(C₃-C₉)-cycloalkyl]NH₂, W—C(═N—SO₂-aryl)NH₂, NH₂, NH—(C₁-C₁₂)-alkyl, N—[(C₁-C₁₂)-alkyl]₂, W—NH—C(═NH)NH₂, W—NH—C(═NH)NH[(C₁-C₆)-alkyl], W—NH—C(═NH)N[(C₁-C₆)-alkyl]₂, W—NH—CO—NH₂, W—NH—CO—NH[(C₁-C₆)-alkyl], W—NH—CO—N[(C₁-C₆)-alkyl], W—NH—CO—NH[(C₃-C₉)-cycloalkyl], W—NH—CO—N[(C₃-C₉)-cycloalkyl]₂, W—NH—CO—NH—[(C₁-C₆)-alkyl]-CO—O—[(C₁-C₆)-alkyl], W—NH—CO—NH—[(C₁-C₆)-alkyl]-CO—NH₂, W—NH—CO—NH—SO₂—(C₁-C₆)-alkyl, W—NH—CO—NH—SO₂-[(C₃-C₉)-cycloalkyl], W—NH—CO—NH—CO—(C₁-C₆)-alkyl, W—NH—CO—NH—CO—[(C₃-C₉)-cycloalkyl], W—NH—C(═NH)—NH—C(═NH)—NH₂, W—NH—C(═NH)—NH—C(═NH)—NH[(C₁-C₆)-alkyl], W—NH—C(═NH)—NH—C(═NH)—N[(C₁-C₆)-alkyl]₂, W—NH—W—SO₂—NH₂, W—NH—W—SO₂—NH[(C₁-C₆)-alkyl], W—NH—W—SO₂—N[(C₁-C₆)-alkyl]₂, W—NH—W—SO₂—NH[(C₃-C₉)-cycloalkyl], W—NH—W—SO₂—N[(C₃-C₉)-cycloalkyl]₂, W—NH—W—SO₂—NH—CO—O[(C₁-C₆)-alkyl], W—NH—W—SO₂—NH—CO—NH₂, W—O—SO₂—NH₂, W—O—W—COOH, W—O—W—CONH₂, W—SO₂—NH₂, W—SO₂—NH[(C₁-C₆)-alkyl], W—SO₂—N[(C₁-C₆)-alkyl]₂, W—SO₂—NH[(C₃-C₉)-cycloalkyl], W—SO₂—N[(C₃-C₉)-cycloalkyl]₂, W—SO₃H, W—NH—W—SO₃H, W—SO₂—NH—CO—NH₂, W—SO₂—NH—CO—NH[(C₁-C₆)-alkyl], W—SO₂—NH—CO—N[(C₁-C₆)-alkyl], W—SO₂—NH—CO—NH[(C₃-C₉)-cycloalkyl], W—SO₂—NH—CO—N[(C₃-C₉)-cycloalkyl], W—P(O)(OH)[O—(C₁-C₆)-alkyl], W—P(O)[O—(C₁-C₆)-alkyl]₂, W—P(O)(OH)(O—CH₂-aryl), W—P(O)(O—CH₂-aryl)₂, W—P(O)(OH)₂, (C₆-C₁₂)-aryl, O—(C₆-C₁₂)-aryl, O—(C₁-C₁₂)-alkylene-(C₆-C₁₂)-aryl, S(O)ₘ—(C₆-C₁₂)-aryl, tri(C₁-C₁₂)-alkylsilyl, where the alkyl has from 1 to 6 carbon atoms;
m is 0, 1, 2;
W is a bond or (C₁-C₆)-alkyl;
R8 is H, (C₁-C₆)-alkyl, where the alkyl group may be substituted by OH, SH, SCH₃, aryl, 4-hydroxyaryl, heteroaryl, NH₂, NH—C(═NH)NH₂, COOH, CO—O(C₁-C₆)-alkyl, CONH₂;
R9 is OH, NH₂, NH—(C₁-C₁₂)-alkyl, N[(C₁-C₁₂)-alkyl]₂, NH—(C₃-C₉)-cycloalkyl, N[(C₃-C₉)-cycloalkyl]₂;
R10 is NH—(C₁-C₆)-alkyl-SO₃H, NH—(C₁-C₆)-alkyl-SO₂NH₂, NH—(C₁-C₆)-alkyl-SO₂—(C₁-C₆)-alkyl, NH—(C₁-C₆)-alkyl-SO₂—(C₃-C₉)-cycloalkyl, NH—(C₁-C₆)-alkyl-SO₂—CF₃, 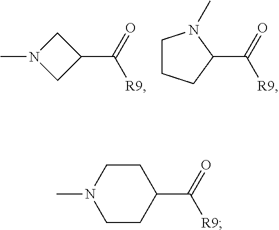
and the physiologically compatible salts thereof.

3. The compound of claim 2, wherein,
R1 is CN or halogen;
R2 is CF₃ or halogen;
A, B are each independently CH, N;
R3, R4 are each independently hydrogen, (C₁-C₁₂)-alkylene-(C₆-C₁₂)-aryl;
R5 is F, Cl, Br, CF₃, SF₅, OCF₃, S(O)₂[(C₁-C₆)-alkyl], (C₁-C₆)-alkyl, OH, —COOH, NH₂, —NH—CO—NH—[(C₁-C₆)-alkyl]-CO—O—[(C₁-C₆)-alkyl], —NH—SO₂—NH₂, —NH—SO₂—NH—CO—O[(C₁-C₆)-alkyl], (C₆-C₁₂)-aryl, O—(C₆-C₁₂)-aryl, O—(C₁-C₁₂)-alkylene-(C₆-C₁₂)-aryl, S(O)ₘ—(C₆-C₁₂)-aryl;
R6, R7 are each independently H, halogen, CN, CF₃, SF₅, OCF₃, S(O)ₘ[(C₁-C₆)-alkyl], S(O)ₘ[(C₃-C₉)-cycloalkyl], S(O)ₘCF₃, (C₁-C₆)-alkyl, (C₁-C₆)-alkoxy, (C₂-C₆)-alkenyl, (C₂-C₆)-alkenyloxy,
(C₂-C₆)-alkynyl, (C₂-C₆)-alkynyloxy, OH, SH, W—COO—[(C₁-C₁₂)-alkyl], —O(C═O)—(C₆-C₁₂)-aryl, W—COOH, W—CONH₂, W—CO—NH[(C₁-C₆)-alkyl], W—CO—N[(C₁-C₆)-alkyl]₂, W—CO—NH[(C₃-C₉)-cycloalkyl], W—CO—N[(C₃-C₉)-cycloalkyl]₂, W—CO—NH—CN, W—CO—NH—CHR8-CO—R9, W—CO—R10, W—CO—NH—C(═NH)NH₂, W—CO—NH—C(═NH)NH[(C₁-C₆)-alkyl], W—CO—NH—C(═NH)N[(C₁-C₆)-alkyl]₂, (C₁-C₈)-acyl, (C₁-C₇)-acyloxy, W—C(═NH)NH₂, W—C(═NH)NHOH, W—C(═N—SO₂—NH₂)NH₂, W—C(═N—SO₂—CF₃)NH₂, W—C[═N—SO₂—(C₁-C₆)-alkyl]NH₂, W—C[═N—SO₂—(C₃-C₉)-cycloalkyl]NH₂, W—C(═N—SO₂-aryl)NH₂, NH₂, NH—(C₁-C₁₂)-alkyl, N—[(C₁-C₁₂)-alkyl]₂, W—NH—C(═NH)NH₂, W—NH—C(═NH)NH[(C₁-C₆)-alkyl], W—NH—C(═NH)N[(C₁-C₆)-alkyl]₂, W—NH—CO—NH₂, W—NH—CO—NH[(C₁-C₆)-alkyl], W—NH—CO—N[(C₁-C₆)-alkyl]₂, W—NH—CO—NH[(C₃-C₉)-cycloalkyl], W—NH—CO—N[(C₃-C₉)-cycloalkyl]₂, W—NH—CO—NH—[(C₁-C₆)-alkyl]-CO—O—[(C₁-C₆)-alkyl], W—NH—CO—NH—[(C₁-C₆)-alkyl]-CO—NH₂, W—NH—CO—NH—SO₂—(C₁-C₆)-alkyl, W—NH—CO—NH—SO₂-[(C₃-C₉)-cycloalkyl], W—NH—CO—NH—CO—(C₁-C₆)-alkyl, W—NH—CO—NH—CO—[(C₃-C₉)-cycloalkyl], W—NH—C(═NH)—NH—C(═NH)—NH₂, W—NH—C(═NH)—NH—C(═NH)—NH[(C₁-C₆)-alkyl], W—NH—C(═NH)—NH—C(═NH)—N[(C₁-C₆)-alkyl]₂, W—NH—W—SO₂—NH₂, W—NH—W—SO₂—NH[(C₁-C₆)-alkyl], W—NH—W—SO₂—N[(C₁-C₆)-alkyl]₂, W—NH—W—SO₂—NH[(C₃-C₉)-cycloalkyl], W—NH—W—SO₂—N[(C₃-C₉)-cycloalkyl]₂, W—NH—W—SO₂—NH—CO—O[(C₁-C₆)-alkyl], W—NH—W—SO₂—NH—CO—NH₂, W—O—SO₂—NH₂, W—O—W—COOH, W—O—W—CONH₂, W—SO₂—NH₂, W—SO₂—NH[(C₁-C₆)-alkyl], W—SO₂—N[(C₁-C₆)-alkyl]₂, W—SO₂—NH[(C₃-C₉)-cycloalkyl], W—SO₂—N[(C₃-C₉)-cycloalkyl]₂, W—SO₃H, W—NH—W—SO₃H, W—SO₂—NH—CO—NH₂, W—SO₂—NH—CO—NH[(C₁-C₆)-alkyl], W—SO₂—NH—CO—N[(C₁-C₆)-alkyl]₂, W—SO₂—NH—CO—NH[(C₃-C₉)-cycloalkyl], W—SO₂—NH—CO—N[(C₃-C₉)-cycloalkyl]₂, W—P(O)(OH)[O—(C₁-C₆)-alkyl], W—P(O)[O—(C₁-C₆)-alkyl]₂, W—P(O)(OH)(O—CH₂-aryl), W—P(O)(O—CH₂-aryl)₂, W—P(O)(OH)₂, (C₆-C₁₂)-aryl, O—(C₆-C₁₂)-aryl, O—(C₁-C₁₂)-alkylene-(C₆-C₁₂)-aryl, S(O)ₘ—(C₆-C₁₂)-aryl, tri(C₁-C₁₂)-alkylsilyl, where the alkyl has from 1 to 6 carbon atoms;
m is 0, 1, 2;
W is a bond or (C₁-C₆)-alkyl;
R8 is H, (C₁-C₆)-alkyl, where the alkyl group may be substituted by OH, SH, SCH₃, aryl, 4-hydroxyaryl, heteroaryl, NH₂, NH—C(═NH)NH₂, COOH, CO—O(C₁-C₆)-alkyl, CONH₂;
R9 is OH, NH₂, NH—(C₁-C₁₂)-alkyl, N[(C₁-C₁₂)-alkyl]₂, NH—(C₃-C₉)-cycloalkyl, N[(C₃-C₉)-cycloalkyl]₂;
R10 is NH—(C₁-C₆)-alkyl-SO₃H, NH—(C₁-C₆)-alkyl-SO₂NH₂, NH—(C₁-C₆)-alkyl-SO₂—(C₁-C₆)-alkyl, NH—(C₁-C₆)-alkyl-SO₂—(C₃-C₉)-cycloalkyl, NH—(C₁-C₆)-alkyl-SO₂—CF₃, 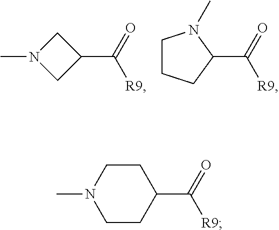
and the physiologically compatible salts thereof.

4. The compound of claim 3, wherein,
R1 is CN or halogen;
R2 is CF₃ or halogen;
A, B are each independently CH, N;
R3, R4 are each independently hydrogen, (C₁-C₁₂)-alkylene-(C₆-C₁₂)-aryl;
R5 is F, Cl, Br, CF₃, SF₅, OCF₃, S(O)₂[(C₁-C₆)-alkyl], (C₁-C₆)-alkyl, OH, —COOH, NH₂, —NH—CO—NH—[(C₁-C₆)-alkyl]-CO—O—[(C₁-C₆)-alkyl], —NH—SO₂—NH₂, —NH—SO₂—NH—CO—O[(C₁-C₆)-alkyl], (C₆-C₁₂)-aryl, O—(C₆-C₁₂)-aryl, O—(C₁-C₁₂)-alkylene-(C₆-C₁₂)-aryl, S(O)ₘ—(C₆-C₁₂)-aryl;
R6, R7 are each independently H, halogen, CF₃, SF₅, OCF₃, S(O)₂[(C₁-C₆)-alkyl], (C₁-C₆)-alkyl, OH, —COOH,
NH₂, —NH—CO—NH—[(C₁-C₆)-alkyl]-CO—O—[(C₁-C₆)-alkyl], —NH—SO₂—NH₂, —NH—SO₂—NH—CO—O[(C₁-C₆)-alkyl], (C₆-C₁₂)-aryl, O—(C₆-C₁₂)-aryl, O—(C₁-C₁₂)-alkylene-(C₆-C₁₂)-aryl, S(O)ₘ—(C₆-C₁₂)-aryl;
and the physiologically compatible salts thereof.

5. The compound of claim 4, wherein,
R1 is CN or halogen;
R2 is CF₃ or halogen;
A is CH;
B is CH, N;
R3, R4 are each independently hydrogen, (C₁-C₁₂)-alkylene-(C₆-C₁₂)-aryl;
R5 is SF₅, OCF₃, S(O)₂[(C₁-C₆)-alkyl], —NH—CO—NH—[(C₁-C₆)-alkyl]-CO—O—[(C₁-C₆)-alkyl], —NH—SO₂—NH₂, —NH—SO₂—NH—CO—O—[(C₁-C₆)-alkyl], O—(C₁-C₁₂)-alkylene-(C₆-C₁₂)-aryl;
R6, R7 are each independently H, halogen, CF₃, SF₅, OCF₃, S(O)₂[(C₁-C₆)-alkyl], (C₁-C₆)-alkyl, OH, —COOH,
NH₂, —NH—CO—NH—[(C₁-C₆)-alkyl]-CO—O—[(C₁-C₆)-alkyl], —NH—SO₂—NH₂, —NH—SO₂—NH—CO—O[(C₁-C₆)-alkyl], (C₆-C₁₂)-aryl, O—(C₆-C₁₂)-aryl, O—(C₁-C₁₂)-alkylene-(C₆-C₁₂)-aryl, S(O)ₘ—(C₆-C₁₂)-aryl;
and the physiologically compatible salts thereof.

6. A compound of the formula
4-[3-(3,5-bis(trifluoromethyl)benzyl)-4,4-dimethyl-2,5-dioxoimidazolidin-1-yl]-2-trifluoromethylbenzonitrile,
4-[4,4-dimethyl-2,5-dioxo-3-(4-trifluoromethoxybenzyl)imidazolidin-1-yl]-2-trifluoromethylbenzonitrile,
4-[4,4-dimethyl-2,5-dioxo-3-(3-trifluoromethylbenzyl)imidazolidin-1-yl]-2-trifluoromethylbenzonitrile,
4-[3-(6-chloropyridin-3-ylmethyl)-4,4-dimethyl-2,5-dioxoimidazolidin-1-yl]-2-trifluoromethylbenzonitrile,
4-[4,4-dimethyl-2,5-dioxo-3-(6-trifluoromethylpyridin-3-ylmethyl)imidazolidin-1-yl]-2-trifluoromethylbenzonitrile,
3-(4-fluoro-3-trifluoromethylphenyl)-5,5-dimethyl-1-(6-trifluoromethylpyridin-3-ylmethyl)imidazolidine-2,4-dione,
3-(4-chloro-3-trifluoromethylphenyl)-5,5-dimethyl-1-(6-trifluoromethylpyridin-3-ylmethyl)imidazolidine-2,4-dione,
1-(3,5-bis(trifluoromethyl)benzyl)-3-(4-fluoro-3-trifluoromethylphenyl)-5,5-dimethyl-imidazolidine-2,4-dione,
1-(3,5-bis(trifluoromethyl)benzyl)-3-(4-chlor-3-trifluoromethylphenyl)-5,5-dimethyl-imidazolidine-2,4-dione,
4-[4,4-dimethyl-2,5-dioxo-3-(3-pentafluorosulfanylbenzyl)imidazolidin-1-yl]-2-trifluoromethylbenzonitrile or
3-(4-fluoro-3-trifluoromethylphenyl)-5,5-dimethyl-1-(3-pentafluorosulfanylbenzyl)-imidazolidine-2,4-dione.

7. A pharmaceutical composition comprising one or more compounds of claim 1.

8. A pharmaceutical composition comprising one or more compounds of claim 6.

9. The pharmaceutical composition of claim 7 and a pharmaceutically acceptable carrier.

10. The pharmaceutical composition of claim 8 and a pharmaceutically acceptable carrier.

11. The pharmaceutical composition of claim 9 and at least one further active ingredient.

12. The pharmaceutical composition of claim 10 and at least one further active ingredient.

13. The pharmaceutical composition claim 11, which comprises, as the further active ingredient, one or more antidiabetics, active hypoglycemic ingredients, HMG-CoA reductase inhibitors, cholesterol absorption inhibitors, PPAR gamma agonists, PPAR alpha agonists, PPAR alpha/gamma agonists, PPAR delta agonists, fibrates, MTP inhibitors, bile acid absorption inhibitors, MTP inhibitors, CETP inhibitors, polymeric bile acid adsorbers, LDL receptor inducers, ACAT inhibitors, antioxidants, lipoprotein lipase inhibitors, ATP citrate lyase inhibitors, squalene synthetase inhibitors, lipoprotein(a) antagonists, HM74A receptor agonists, lipase inhibitors, insulins, sulfonylureas, biguanides, meglitinides, thiazolidinediones, α-glucosidase inhibitors, active ingredients which act on the ATP-dependent potassium channel of the beta cells, glycogen phosphorylase inhibitors, glucagon receptor antagonists, activators of glucokinase, inhibitors of gluconeogenesis, inhibitors of fructose 1,6-biphosphatase, modulators of glucose transporter 4, inhibitors of glutamine:fructose-6-phosphate amidotransferase, inhibitors of dipeptidylpeptidase IV, inhibitors of 11-beta-hydroxysteroid dehydrogenase 1, inhibitors of protein tyrosine phosphatase 1B, modulators of the sodium-dependent glucose transporter 1 or 2, modulators of GPR40, inhibitors of hormone-sensitive lipase, inhibitors of acetyl-CoA carboxylase, inhibitors of phosphoenolpyruvate carboxykinase, inhibitors of glycogen synthase kinase-3 beta, inhibitors of protein kinase C beta, endothelin-A receptor antagonists, inhibitors of I kappaB kinase, modulators of the glucocorticoid receptor, CART agonists, NPY agonists, MC4 agonists, orexin antagonists, H3 antagonists, TNF antagonists, CRF antagonists, CRF BP antagonists, urocortin agonists, β3 agonists, CB1 receptor antagonists, MSH (melanocyte-stimulating hormone) agonists, MCH antagonists, CCK agonists, serotonin reuptake inhibitors, mixed serotoninergic and noradrenergic compounds, 5HT modulators, bombesin agonists, galanin antagonists, growth hormones, growth hormone-releasing compounds, TRH agonists, decoupling protein 2 or 3 modulators, leptin agonists, DA agonists (bromocriptin, doprexin), lipase/amylase inhibitors, PPAR modulators, RXR modulators or TR-β agonists or amphetamines.

14. The pharmaceutical composition of claim 12, which comprises, as the further active ingredient, one or more antidiabetics, active hypoglycemic ingredients, HMG-CoA reductase inhibitors, cholesterol absorption inhibitors, PPAR gamma agonists, PPAR alpha agonists, PPAR alpha/gamma agonists, PPAR delta agonists, fibrates, MTP inhibitors, bile acid absorption inhibitors, MTP inhibitors, CETP inhibitors, polymeric bile acid adsorbers, LDL receptor inducers, ACAT inhibitors, antioxidants, lipoprotein lipase inhibitors, ATP citrate lyase inhibitors, squalene synthetase inhibitors, lipoprotein(a) antagonists, HM74A receptor agonists, lipase inhibitors, insulins, sulfonylureas, biguanides, meglitinides, thiazolidinediones, α-glucosidase inhibitors, active ingredients which act on the ATP-dependent potassium channel of the beta cells, glycogen phosphorylase inhibitors, glucagon receptor antagonists, activators of glucokinase, inhibitors of gluconeogenesis, inhibitors of fructose 1,6-biphosphatase, modulators of glucose transporter 4, inhibitors of glutamine:fructose-6-phosphate amidotransferase, inhibitors of dipeptidylpeptidase IV, inhibitors of 11-beta-hydroxysteroid dehydrogenase 1, inhibitors of protein tyrosine phosphatase 1B, modulators of the sodium-dependent glucose transporter 1 or 2, modulators of GPR40, inhibitors of hormone-sensitive lipase, inhibitors of acetyl-CoA carboxylase, inhibitors of phosphoenolpyruvate carboxykinase, inhibitors of glycogen synthase kinase-3 beta, inhibitors of protein kinase C beta, endothelin-A receptor antagonists, inhibitors of I kappaB kinase, modulators of the glucocorticoid receptor, CART agonists, NPY agonists, MC4 agonists, orexin antagonists, H3 antagonists, TNF antagonists, CRF antagonists, CRF BP antagonists, urocortin agonists, β3 agonists, CB1 receptor antagonists, MSH (melanocyte-stimulating hormone) agonists, MCH antagonists, CCK agonists, serotonin reuptake inhibitors, mixed serotoninergic and noradrenergic compounds, 5HT modulators, bombesin agonists, galanin antagonists, growth hormones, growth hormone-releasing compounds, TRH agonists, decoupling protein 2 or 3 modulators, leptin agonists, DA agonists (bromocriptin, doprexin), lipase/amylase inhibitors, PPAR modulators, RXR modulators or TR-β agonists or amphetamines.

15. A method of treating metabolic syndrome, diabetes, obesity, weight reduction, nicotine dependence, alcohol dependence, CNS disorders, schizophrenia, Alzheimer's or polycystic ovary syndrome (PCOS) comprising administering a pharmaceutically acceptable amount of the compound of claim 1.

16. A method of treating metabolic syndrome, diabetes, obesity, weight reduction, nicotine dependence, alcohol dependence, CNS disorders, schizophrenia, Alzheimer's or polycystic ovary syndrome (PCOS) comprising administering a pharmaceutically acceptable amount of the compound of claim 6.
